(19) United States
(12) Patent Application Publication
(10) Pub. No.: US 2026/0275363 A1
(43) Pub. Date: Sep. 17, 2026

(54) PHAGE DEFENCE GENES FOR STARTER STRAINS

(71) Applicant: DSM IP Assets B.V., Maastricht (NL)

(72) Inventors: Van Sinderen DOUWE, Kaiseraugst (CH); Jennifer MAHONY, Kaiseraugst (CH); Andriana GRAFAKOU, Kaiseraugst (CH); Matthias Hermann BECK, Kaiseraugst (CH); Cas MOSTERD, Kaiseraugst (CH); Noël VAN PEIJ, Kaiseraugst (CH); Irma VAN RIJSWIJCK, Kaiseraugst (CH); Paul DE WAAL, Kaiseraugst (CH); Paul KLAASSEN, Kaiseraugst (CH)

(21) Appl. No.: 19/469,111

(22) PCT Filed: Mar. 28, 2024
(86) PCT No.: PCT/EP2024/058584

§ 371 Date: Sep. 25, 2025

(30) Foreign Application Priority Data
Mar. 31, 2023 (GR) 20230100275
Apr. 6, 2023 (EP) 23166918.5
Jun. 27, 2023 (EP) 23181850.1

(51) Int. Cl. C12N 15/74; A23C 9/123; C07K 14/195; C12N 1/20; C12N 15/10; C12Q 1/04; C12Q 1/68; C12R 1/225; C12R 1/46

(52) U.S. Cl. CPC C12N 15/746; A23C 9/123; C07K 14/195; C12N 1/20; C12N 15/102; C12Q 1/04; C12Q 1/68; C12N 2800/101; C12R 2001/225; C12R 2001/46

(57) ABSTRACT

The invention relates to the field of fermentation technology. The invention specifically relates to genes that are useful for protecting bacterial strains from phage infections, and to bacterial strains comprising such genes. Such strains can be used in methods to produce a fermented food product such as a fermented milk product. The invention also relates to food products produced using such strain or composition.

### FIELD

The invention relates to the field of fermentation technology. The invention specifically relates to genes that are useful for protecting bacterial strains from phage infections, and to bacterial strains comprising such genes. Such strains can be used in methods to produce a fermented food product such as a fermented milk product. The invention also relates to food products produced using such strain or composition.

### BACKGROUND

Lactococcus lactis and Lactococcus cremoris (formerly assigned as two subspecies of Lactococcus lactis) represent bacterial species of substantial economic and industrial importance due to their extensive use in the production of fermented food products, such as cheese, yogurt and sauerkraut, a practice that goes back centuries (Mills et al., 2006; Cavanagh et al., 2015). These two lactococcal species represent Gram-positive, non-spore forming, micro-aerophilic coccoid bacteria, that belong to the lactic acid bacteria (LAB) (Makarova et al., 2006), and that enjoy a so-called generally recognized as safe (GRAS) status according to the Food and Drug Administration through their long history of safe application in foods (FDA, 2010). As functional constituents of the dairy starter culture, their primary role in dairy fermentations is to produce lactic acid, among other antimicrobial compounds such as bacteriocins, and to degrade the milk proteins, contributing to microbial stability and organoleptic characteristics of the final product (Ross et al., 2000; Wouters et al., 2002).

However, also many other Lactococcus strains exist. Some (parts) of naturally occurring (non man-made) Lactococcus strains have been analyzed, such as for example the “Lactococcus piscium MKFS47 genome assembly L_piscium, plasmid: II” (ncbi GenBank: LN774770.1); and the “Lactococcus garvieae strain LG791 plasmid unnamed1”: (ncbi GenBank: CP071292.1).

The widespread and intensive application of lactococcal strains is associated with the emergence of host-specific bacteriophages which are ubiquitous in the (non-sterile) dairy environment and which represent a persistent challenge to fermentation processes (Hayes et al., 2017). Phage infection may cause lysis of the starter culture, resulting in delayed or even failed fermentations with negative economic consequences for producers (Garneau & Moineau, 2011). Lactococcal phages have been classified into eleven distinct groups (Deveau et al., 2006; Zrelovs et al., 2021). Among these, three phage groups are particularly prevalent and problematic in modern, large-scale dairy fermentation plants, i.e. the skunaviruses (formerly called the 936 group phages), ceduoviruses (formerly termed the c2 group phages), and the P335 phage group. These three groups incorporate either virulent phages (phages of the genera Skunavirus & Ceduovirus) or both virulent and temperate phages (P335 group) (Mahony et al., 2016).

To defend themselves against phages, bacteria have adopted multiple resistance strategies that interfere with different stages of the phage life cycle, such as preventing phage adsorption or phage DNA injection, restriction of incoming phage nucleic acids (Restriction-modification (RM) & CRISPR-Cas systems) and abortive infection (Abi) (Chopin et al., 2005; Labrie et al., 2010). Abi systems are considered to represent altruistic defence mechanisms. Upon infection the cell activates the Abi system which then blocks an essential cellular activity thereby preventing phage proliferation and subsequent infection of neighbouring cells (Lopatina et al., 2020). Initially, the phage binds to a specific receptor on the cell surface and injects its genome into the host cell cytoplasm during or after which the Abi system is activated resulting in the interruption of the phage cycle (Chopin et al., 2005). It has been shown that Abi systems can be triggered by DNA-protein complexes of phage genome replication (Snyder, 1995), phage-encoded proteins (Schmitt & Molineux, 1991; Bidnenko et al., 2009) or phage-mediated shut-off of host gene expression (Koga et al., 2011), while activation can lead to cell death by degrading simultaneously phage and host DNA/RNA (Lau et al., 2020; Otsuka & Yonesaki, 2012) or by inactivating host protein synthesis (Bingham et al., 2000) or by damaging/disturbing the cell membrane (Schmitt & Molineux, 1991; Durmaz & Klaenhammer, 2007).

Abi systems are highly diverse in L. lactis & L. cremoris (Chopin et al., 2005). To date, 23 genetically distinct and mechanistically diverse lactococcal Abi mechanisms have been reported, designated AbiA, AbiB, AbiC, AbiD, AbiD1, AbiE, AbiF, AbiG, AbiH, Abil, AbiJ, AbiK, AbiL, AbiN, AbiO, AboP, AbiQ, AbiR, AbiS, AbiT, AbiU, AbiV and AbiZ (Ainsworth et al., 2014b). The majority of currently defined systems are plasmid-encoded, typically by one or two genes and with diverse modes of action interfering with critical stages of the phage cycle, including DNA replication, transcription and protein production (Makarova et al., 2006; Ainsworth et al., 2014b).

The technological necessity for starter strains which are endowed with improved phage-resistance, and which at the same time can be utilised to meet consumer demands for unique products with certain characteristics (Mills et al., 2010; Cavanagh et al., 2015), highlights the importance of creating novel strains with all the above-mentioned features.

Phage-resistance systems can be, however, bypassed through phage-specific mutations, resulting in delayed fermentations and downstream economic and food quality consequences. This highlights the necessity of expanding the known arsenal of defence systems by identifying novel phage resistance systems, such as novel Abi systems with different modes of actions, that may be combined for the ongoing and continuous arms race against phages. Although genetically modified organisms (GMOs) may address this problem to some extent, their use by the food industry is subject to tight regulatory constraints and considerable consumer opposition (Pedersen et al., 2005). Additionally, also the use of GMOs ultimately will require the expansion of the known arsenal of defence systems with novel phage resistance systems. There is a need for starter strains with improved phage resistance and phage resistance, preferably based on novel systems and mode of action. There thus remains a need for new methods of improving the phage resistance of starter strains. There is further a need for food-grade means to improve phage resistance.

### SUMMARY

As indicated above, phage infection may cause lysis of the starter culture, resulting in delayed or even failed fermentations with negative economic consequences for producers. This may cause loss of valuable food raw materials and products. The persistent challenge of (bacterio)phages in dairy fermentations requires the development of starter cultures with enhanced phage-resistance characteristics, which the inventors have achieved through the recruitment of novel natural phage defence systems as claimed herein.

Accordingly, the invention provides a nucleic acid construct comprising a first nucleotide sequence (herein also referred to as “first sequence”) that has at least 80% sequence identity, preferably at least 90% sequence identity, more preferably at least 95% sequence identity, even more preferably at least 99% sequence identity, to any one of SEQ ID NO: 60, SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 55, SEQ ID NO: 56, SEQ ID NO: 57, SEQ ID NO: 58, SEQ ID NO: 59, SEQ ID NO: 61, SEQ ID NO: 62, SEQ ID NO: 63, SEQ ID NO: 64, SEQ ID NO; 65, SEQ ID NO: 66 and/or SEQ ID NO: 67 (herein also referred to as “SEQ ID NOs: 1-8 and/or 55-67”). Most preferably the first sequence has at least 100% sequence identity to any one of SEQ ID NOs: 60, 1-8 and/or 55-59 and/or 61-67, and preferably it is operably linked to a promoter, preferably the native promoter, and/or terminator. The term “native promoter” is to be construed as the promotor that is associated with the sequence in a naturally occurring isolate.

Preferably, the nucleic acid construct according to the invention encodes a polypeptide with an amino acid sequence that has at least 80% sequence identity, preferably at least 90% sequence identity, more preferably at least 95% sequence identity, even more preferably at least 99% sequence identity to the amino acid sequence as set forth in any one of SEQ ID NOs: 73, 21-28 and/or 68-72 and/or 74-80.

Preferably, the first nucleotide sequence is an isolated nucleotide sequence, meaning that it has been isolated from nature by man. Alternatively, the nucleotide sequence can be constructed synthetically.

In some embodiments, the first sequence is followed by a second nucleotide sequence (herein also referred to as “second sequence”) that has at least 80% sequence identity, preferably at least 90% sequence identity, more preferably at least 95% sequence identity, even more preferably at least 99% sequence identity, and most preferably 100% sequence identity, to any one of SEQ ID NOS: 1-8 and/or 55-67, or by a second sequence which encodes a polypeptide with an amino acid sequence that has at least 80% sequence identity to the amino acid sequence as set forth in any one of SEQ ID NOS: 21-28 and/or 68-80. In some embodiments, the nucleic acid construct comprises a first and a second nucleic acid sequence. Preferably, the first and the second sequence have at least 80% sequence identity, preferably at least 90% sequence identity, more preferably at least 95% sequence identity, even more preferably at least 99% sequence identity, and most preferably 100% sequence identity, with respectively SEQ ID NOS: 5 and 6, or the first and second sequence have at least 80% sequence identity, preferably at least 90% sequence identity, more preferably at least 95% sequence identity, even more preferably at least 99% sequence identity, and most preferably 100% sequence identity, with respectively SEQ ID NOs: 7 and 8. Preferably, the first and the second sequence have at least 80% sequence identity, preferably at least 90% sequence identity, more preferably at least 95% sequence identity, even more preferably at least 99% sequence identity, and most preferably 100% sequence identity, with respectively SEQ ID NOS: 55 and 56, or the first and second sequence have at least 80% sequence identity, preferably at least 90% sequence identity, more preferably at least 95% sequence identity, even more preferably at least 99% sequence identity, and most preferably 100% sequence identity, with respectively SEQ ID NOs: 65 and 66.

Preferably, the first and second sequence preferably encode a polypeptide with an amino acid sequence that has at least 80% sequence identity to the amino acid sequence as set forth in SEQ ID NO:s 25 and 26, 27 and 28, 68 and 69, or 78 and 79, respectively.

Preferably, the second nucleotide sequence is an isolated nucleotide sequence, meaning that it has been isolated from nature by man. Alternatively, the nucleotide sequence can be constructed synthetically.

In some embodiments, the first and second sequences are followed by a third nucleotide sequence (herein also referred to as “third sequence”) that has at least 80% sequence identity, preferably at least 90% sequence identity, more preferably at least 95% sequence identity, even more preferably at least 99% sequence identity, and most preferably 100% sequence identity, to any one of SEQ ID NOS: 1-8 and/or 55-67. In some embodiments, the nucleic acid construct comprises a first, second and a third nucleic acid sequence.

Preferably, the first, second and third sequence have at least 80% sequence identity, preferably at least 90% sequence identity, more preferably at least 95% sequence identity, even more preferably at least 99% sequence identity, and most preferably 100% sequence identity, with respectively SEQ ID NOs: 57, 58 and 59, or the first, second and third sequence have at least 80% sequence identity, preferably at least 90% sequence identity, more preferably at least 95% sequence identity, even more preferably at least 99% sequence identity, and most preferably 100% sequence identity, with respectively SEQ ID NOs: 62, 63 and 64.

Preferably, the first second and third sequence encode a polypeptide with an amino acid sequence that has at least 80% sequence identity to the amino acid sequence as set forth in SEQ ID NOs: 70, 71 and 72, or 75, 76 and 77, respectively.

Preferably, the third nucleotide sequence is an isolated nucleotide sequence, meaning that it has been isolated from nature by man. Alternatively, the nucleotide sequence can be constructed synthetically.

In a further aspect, the invention also provides a nucleic acid construct comprising a nucleotide sequence that has at least 80% sequence identity, preferably at least 90% sequence identity, more preferably at least 95% sequence identity, even more preferably at least 99% sequence identity, and most preferably 100% sequence identity, to SEQ ID NO: 9, which suitably comprises a combination of SEQ ID NO: 5 and SEQ ID NO: 6, or a nucleic acid construct comprising a nucleotide sequence that has at least 80% sequence identity, preferably at least 90% sequence identity, more preferably at least 95% sequence identity, even more preferably at least 99% sequence identity, and most preferably 100% sequence identity, to SEQ ID NO: 10, which suitably comprises a combination of SEQ ID NO: 7 and SEQ ID NO: 8.

This aspect also provides a nucleic acid construct comprising a nucleotide sequence that has at least 80% sequence identity, preferably at least 90% sequence identity, more preferably at least 95% sequence identity, even more preferably at least 99% sequence identity, and most preferably 100% sequence identity to SEQ ID NO: 55 and further comprising a nucleotide sequence that has at least 80% sequence identity, preferably at least 90% sequence identity, more preferably at least 95% sequence identity, even more preferably at least 99% sequence identity, and most preferably 100% sequence identity to SEQ ID NO: 56.

This aspect also provides a nucleic acid construct comprising a nucleotide sequence that has at least 80% sequence identity, preferably at least 90% sequence identity, more preferably at least 95% sequence identity, even more preferably at least 99% sequence identity, and most preferably 100% sequence identity to SEQ ID NO: 65 and further comprising a nucleotide sequence that has at least 80% sequence identity, preferably at least 90% sequence identity, more preferably at least 95% sequence identity, even more preferably at least 99% sequence identity, and most preferably 100% sequence identity to SEQ ID NO: 66.

This aspect also provides a nucleic acid construct comprising a nucleotide sequence that has at least 80% sequence identity, preferably at least 90% sequence identity, more preferably at least 95% sequence identity, even more preferably at least 99% sequence identity, and most preferably 100% sequence identity to SEQ ID NO: 57, further comprising a nucleotide sequence that has at least 80% sequence identity, preferably at least 90% sequence identity, more preferably at least 95% sequence identity, even more preferably at least 99% sequence identity, and most preferably 100% sequence identity to SEQ ID NO: 58, and further comprising a nucleotide sequence that has at least 80% sequence identity, preferably at least 90% sequence identity, more preferably at least 95% sequence identity, even more preferably at least 99% sequence identity, and most preferably 100% sequence identity to SEQ ID NO: 59.

This aspect also provides a nucleic acid construct comprising a nucleotide sequence that has at least 80% sequence identity, preferably at least 90% sequence identity, more preferably at least 95% sequence identity, even more preferably at least 99% sequence identity, and most preferably 100% sequence identity to SEQ ID NO: 62, further comprising a nucleotide sequence that has at least 80% sequence identity, preferably at least 90% sequence identity, more preferably at least 95% sequence identity, even more preferably at least 99% sequence identity, and most preferably 100% sequence identity to SEQ ID NO: 63, and further comprising a nucleotide sequence that has at least 80% sequence identity, preferably at least 90% sequence identity, more preferably at least 95% sequence identity, even more preferably at least 99% sequence identity, and most preferably 100% sequence identity to SEQ ID NO: 64.

This aspect also provides a nucleic acid construct comprising a nucleotide sequence that encodes a polypeptide with an amino acid sequence that has at least 80% sequence identity, preferably at least 90% sequence identity, more preferably at least 95% sequence identity, even more preferably at least 99% sequence identity, and most preferably 100% sequence identity to SEQ ID NO: 25 and further comprising a nucleotide sequence that that encodes a polypeptide with an amino acid sequence that has at least 80% sequence identity, preferably at least 90% sequence identity, more preferably at least 95% sequence identity, even more preferably at least 99% sequence identity, and most preferably 100% sequence identity to SEQ ID NO: 26.

This aspect also provides a nucleic acid construct comprising a nucleotide sequence that encodes a polypeptide with an amino acid sequence that has at least 80% sequence identity, preferably at least 90% sequence identity, more preferably at least 95% sequence identity, even more preferably at least 99% sequence identity, and most preferably 100% sequence identity to SEQ ID NO: 27 and further comprising a nucleotide sequence that that encodes a polypeptide with an amino acid sequence that has at least 80% sequence identity, preferably at least 90% sequence identity, more preferably at least 95% sequence identity, even more preferably at least 99% sequence identity, and most preferably 100% sequence identity to SEQ ID NO: 28.

This aspect also provides a nucleic acid construct comprising a nucleotide sequence that encodes a polypeptide with an amino acid sequence that has at least 80% sequence identity, preferably at least 90% sequence identity, more preferably at least 95% sequence identity, even more preferably at least 99% sequence identity, and most preferably 100% sequence identity to SEQ ID NO: 68 and further comprising a nucleotide sequence that that encodes a polypeptide with an amino acid sequence that has at least 80% sequence identity, preferably at least 90% sequence identity, more preferably at least 95% sequence identity, even more preferably at least 99% sequence identity, and most preferably 100% sequence identity to SEQ ID NO: 69.

This aspect also provides a nucleic acid construct comprising a nucleotide sequence that encodes a polypeptide with an amino acid sequence that has at least 80% sequence identity, preferably at least 90% sequence identity, more preferably at least 95% sequence identity, even more preferably at least 99% sequence identity, and most preferably 100% sequence identity to SEQ ID NO: 78 and further comprising a nucleotide sequence that encodes a polypeptide with an amino acid sequence that has at least 80% sequence identity, preferably at least 90% sequence identity, more preferably at least 95% sequence identity, even more preferably at least 99% sequence identity, and most preferably 100% sequence identity to SEQ ID NO: 79.

This aspect also provides a nucleic acid construct comprising a nucleotide sequence that that encodes a polypeptide with an amino acid sequence has at least 80% sequence identity, preferably at least 90% sequence identity, more preferably at least 95% sequence identity, even more preferably at least 99% sequence identity, and most preferably 100% sequence identity to SEQ ID NO: 70, further comprising a nucleotide sequence that that encodes a polypeptide with an amino acid sequence has at least 80% sequence identity, preferably at least 90% sequence identity, more preferably at least 95% sequence identity, even more preferably at least 99% sequence identity, and most preferably 100% sequence identity to SEQ ID NO: 71, and further comprising a nucleotide sequence that that encodes a polypeptide with an amino acid sequence has at least 80% sequence identity, preferably at least 90% sequence identity, more preferably at least 95% sequence identity, even more preferably at least 99% sequence identity, and most preferably 100% sequence identity to SEQ ID NO: 72.

This aspect also provides a nucleic acid construct comprising a nucleotide sequence that encodes a polypeptide with an amino acid sequence that has at least 80% sequence identity, preferably at least 90% sequence identity, more preferably at least 95% sequence identity, even more preferably at least 99% sequence identity, and most preferably 100% sequence identity to SEQ ID NO: 75, further comprising a nucleotide sequence that encodes a polypeptide with an amino acid sequence that has at least 80% sequence identity, preferably at least 90% sequence identity, more preferably at least 95% sequence identity, even more preferably at least 99% sequence identity, and most preferably 100% sequence identity to SEQ ID NO: 76, and further comprising a nucleotide sequence that encodes a polypeptide with an amino acid sequence that has at least 80% sequence identity, preferably at least 90% sequence identity, more preferably at least 95% sequence identity, even more preferably at least 99% sequence identity, and most preferably 100% sequence identity to SEQ ID NO: 77.

Preferably the construct encodes a polypeptide that confers improved phage resistance to a lactococcal host cell. Preferably, the construct expresses in a lactococcal host cell a polypeptide that confers improved phage resistance to the lactococcal host cell.

Also provided is a vector, preferably a plasmid, comprising the nucleic acid construct described above, wherein the plasmid is preferably a conjugative plasmid, more preferably a lactococcal conjugative plasmid.

Also provided is a host cell comprising this nucleic acid construct, vector or this plasmid. Preferably, such host cell is an isolated host cell. Preferably the host cell is obtained by conjugation, transformation, electro transformation, electroporation, recombineering, mutagenesis, or genome editing, and subsequent selection for improved phage resistance. However, a naturally occurring host cell comprising a nucleic acid construct according to the invention may conveniently be used for the production of Lactococcus strain with improved phage resistance.

Accordingly, there is provided for a method for the production of a Lactococcus strain with improved phage resistance, the method comprising:
i) providing a host cell comprising a nucleic acid construct according to the invention;
ii) providing a recipient Lactococcus strain;
iii) contacting the host cell comprising the nucleic acid construct according to the invention with the recipient Lactococcus strain to obtain a transconjugant
iv) selecting for a transconjugant comprising the conjugative plasmid to identify a strain with improved phage resistance; and optionally
v) isolating the strain with improved phage resistance.

Also provided is use of a nucleic acid construct or a vector, preferably a plasmid as described above in the production of a Lactococcus strain with improved phage resistance. For such use, the nucleic acid construct or a vector, preferably a plasmid is contacted with the host cell, such that the nucleic acid construct or a vector, preferably a plasmid is taken up by the host cell, conferring improved phage resistance upon expression of a nucleic acid construct according to the invention. The person skilled in the art knows the recombinant and non-recombinant techniques that can be used to have a nucleic acid, vector of plasmid taken up by a cell.

Also provided is a method for the production of a Lactococcus strain with improved phage resistance, the method comprising:
i) providing a recipient Lactococcus strain;
ii) contacting the recipient Lactococcus strain with a donor Lactococcus strain, wherein the donor strain comprises a conjugative plasmid that comprises:
   a nucleotide sequence that has at least 80% sequence identity to any one of SEQ ID NOs: 1-8 and/or 55-67, to obtain a transconjugant; or
   a nucleotide sequence that has at least 80% sequence identity to SEQ ID NO: 9 or SEQ ID NO: 10, or
   a nucleotide sequence that encodes a polypeptide with an amino acid sequence that has at least 80% sequence identity to the amino acid sequence as set forth in any one of SEQ ID NOs: 21-28 and/or 68-80,
to obtain a transconjugant;
iii) selecting for a transconjugant comprising the conjugative plasmid to identify a strain with improved phage resistance; and optionally
iv) isolating the strain with improved phage resistance.

Also provided is the isolated Lactococcus strain obtainable by this method.

Also provided is a starter culture for the production of a fermented food product, comprising such an isolated strain or a multiplicity of distinct isolated strains according to the invention, wherein the starter culture further preferably comprises an excipient such as a cryoprotectant, a lyoprotectant, an antioxidant, and/or a nutrient, wherein the starter culture is preferably frozen, lyophilized, spray-dried, vacuum-dried, air dried, tray dried, or in liquid form, wherein the starter culture preferably further comprises a further Lactococcus strain or a Lactobacillus strain or a Streptococcus strain, preferably a further Lactococcus strain or a Lactobacillus helveticus strain or a Streptococcus thermophilus strain, most preferably the starter culture further comprises a further Lactococcus strain, or a Lactobacillus helveticus strain, or a Streptococcus thermophilus strain, or both a Lactobacillus helveticus strain and a Streptococcus thermophilus strain. Provided is the use of such an isolated strain, or of such a starter culture, for the production of a fermented food product.

Also provided is a method for the production of a fermented food product, the method comprising the step of fermenting a substrate with an isolated strain or with the starter culture described above, wherein the method is preferably performed in a non-sterile environment. Also provided is a fermented food product obtainable by such a method, wherein the fermented food product is preferably a fermented milk product. In this method, multiple distinct isolated strains as described herein are preferably used, wherein the multiple distinct strains each comprise a different nucleic acid construct according to the invention, and/or a different plasmid according to the invention, or a different set of nucleic acid constructs according to the invention, and/or different plasmids according to the invention. The method set forth hereinabove may be repeated at least once, wherein in each round of the method multiple distinct isolated strains are used so that a subsequent round will comprises a strain or starter culture with one or more different Abi's compared to the previous round. Such strains, may in addition to the novel Abi's according to the invention also comprise known Abi's. In such a way, a starter culture may comprise multiple distinct strains with different Abi systems and a so-called rotation scheme of multiple starter cultures may comprise multiple distinct strains with different Abi systems, as is known by the person skilled in the art with respect to dairy starter culture products.

Also provided is a fermented food product obtainable by a method as set forth herein above, wherein the fermented food product is preferably a fermented milk product.

Also provided is a fermented food product, preferably a fermented milk product, wherein the food product comprises a host cell comprising a nucleic acid construct, vector or plasmid according to the invention.

### DESCRIPTION OF THE FIGURES

The invention is illustrated by the following figures:

FIG. 1 provides an overview of the cloning approach. FIG. 1A illustrates the cloning of potential antiviral systems in high copy vector pNZ44. FIG. 1B illustrates the cloning of potential antiviral systems in low copy, nisin-inducible vector pPTPi.

FIG. 2 illustrates the results of a spot test on plate to test for phage resistance using
A: phage 936 phage (dilution sectors on plate) and L. cremoris 3107 strains with a vector as indicated, i.e. respectively pNZ44 (bottom petri-dish in top picture “A”), pNZ44AbiM (most right petri-dish in top picture “A”), pNZ44-AbiW (most left petri-dish in top picture “A”); and
B. phage P335 phage (dilution sectors on plate) and L. cremoris 3107 strains with a vector as indicated, i.e. respectively pNZ44 (bottom petri-dish in bottom picture “B”), pNZ44AbiM (most left petri-dish in bottom picture “B”), pNZ44-AbiW (most right petri-dish in bottom picture “B”).

Each sector indicated on the plates shown in FIGS. 2A and 2B represents a dilution factor of the spotted phage lysate; the left top sector of each plate represents the lowest dilution factor and the latter increases with ten-fold each sector following counterclockwise to the highest dilution factor in the top right sector.

FIG. 3 illustrates the Efficiency of Plaquing (EOP) and provides a summary of the results of novel and existing phage systems using various lactococcal strains, phage types and different phages as indicated. Skunaviruses: sk1, 712, P2 and JJ50 were tested with L. cremoris NZ9000, bIL66, bIL70, P008, P113G and 340 with L. lactis IL1403, and 66901 and 62601 were tested with L. cremoris 3107. Ceduovirus: C2 was tested with L. cremoris NZ9000. P335-group: P335 was tested with L. lactis IL1403, and TP901-1, LC3, Dub35A and 63301 were tested with L. cremoris 3107. P087-group: phage P087 was tested with L. cremoris 3107. On the left side of the Figure, the novel and known Abi-systems are indicated. The EOP reduction is expressed as a greyscale: the darker the grey tint, the higher the EOP reduction (see gray scale in the Figure). An asterisk indicates plaque size reduction.

FIG. 4 illustrates the plasmid map of LL81569_pD, with position of of LL81569_pD29 ORF (AbiW) indicated.

FIG. 5 illustrates the plasmid map of LL69075_pB, with position of LL69075_pB49 (AbiX) ORF indicated. Two additionally present and known Abi systems (AbiA & AbiZ) on this plasmid are indicated as well.

FIG. 6 illustrates the plasmid map of LL66563_pD, with position of LL66563_pD54 & pD53 ORF's (AbiYᵢ & AbiYᵢᵢ) indicated.

FIG. 7 illustrates the plasmid map of LL75843_pE, with position of pLL75843_pE085 ORF (AbiZA) indicated.

FIG. 8 illustrates the plasmid map of LL75953_pC, with position of pLL75953_pC024 & pC025 ORF's (Abi27a&b) indicated.

FIG. 9 illustrates the results of absorption and transduction assays. Adsorption percentages of phages sk1 (panel A) and c2 (panel B) to L. cremoris NZ9000 strains carrying antiphage systems compared to control strains L. cremoris NZ9000::pNZ44 and L. cremoris NZ9000::pPTPi induced at 1 ng/ml (panel C I) or 10 ng/mL nisin (panel C II). Transduction frequencies in CFUs/mL for L. cremoris NZ9000::pNZ44 derivatives transduced with PPTPL-sk1 cos (panel D) or pPTPi-c2 cos (panel E) or L. cremoris NZ9000::pPTPi derivatives induced at 1 ng/ml (panel F I) or 10 ng/ml (panel F II) transduced with pPTPI-sk1 cos. Experiments were performed in biological triplicate and data are presented as means±standard deviation (SD). Asterisk marks statistically significant differences in transduction frequency (unpaired t-test, P-value <0.05).

FIG. 10 illustrates the results of lysis in broth assays. Lysis-in-broth graphs for L. cremoris NZ9000::pNZ44 and various antiphage system-expressing derivatives employing phages sk1 (panel A) and c2 (panel B), as well as for L. cremoris::pPTPi and various antiphage-expressing derivatives employing phage sk1 (panel C).

### BRIEF DESCRIPTION OF THE SEQUENCE LISTING

This application contains a Sequence Listing in computer readable form, which is incorporated herein by reference. An overview is provided by Table 1 below:

TABLE 1
Overview of sequence listings:

| SEQ ID NO: | Name (Strain/Plasmid/ORF) | Type | Organism |
|---|---|---|---|
| SEQ ID NO: 1 | AbiM (LL79476_pB52) | other DNA | Lactococcus lactis |
| SEQ ID NO: 2 | AbiW (LL81569_pD29) | other DNA | Lactococcus lactis |
| SEQ ID NO: 3 | AbiX (LL69075_pB49) | other DNA | Lactococcus lactis |
| SEQ ID NO: 4 | AbiZA (LL75843_pE085) | other DNA | Lactococcus lactis |
| SEQ ID NO: 5 | AbiYi (LL66563_pD54) | other DNA | Lactococcus lactis |
| SEQ ID NO: 6 | AbiYii (LL66563_pD53) | other DNA | Lactococcus lactis |
| SEQ ID NO: 7 | Abi27a (LL75953_pC24) | other DNA | Lactococcus lactis |
| SEQ ID NO: 8 | Abi27b (LL75953_pC25) | other DNA | Lactococcus lactis |
| SEQ ID NO: 9 | AbiY combined | other DNA | Lactococcus lactis |
| SEQ ID NO: 10 | Abi27(a/b) combined | other DNA | Lactococcus lactis |
| SEQ ID NO: 11 | RNA for AbiM | transcribed RNA | Lactococcus lactis |
| SEQ ID NO: 12 | RNA for AbiW | transcribed RNA | Lactococcus lactis |
| SEQ ID NO: 13 | RNA for AbiX | transcribed RNA | Lactococcus lactis |
| SEQ ID NO: 14 | RNA for AbiZA | transcribed RNA | Lactococcus lactis |
| SEQ ID NO: 15 | RNA for AbiYi | transcribed RNA | Lactococcus lactis |
| SEQ ID NO: 16 | RNA for AbiYii | transcribed RNA | Lactococcus lactis |
| SEQ ID NO: 17 | RNA for Abi27a | transcribed RNA | Lactococcus lactis |
| SEQ ID NO: 18 | RNA for Abi27b | transcribed RNA | Lactococcus lactis |
| SEQ ID NO: 19 | RNA for AbiY | transcribed RNA | Lactococcus lactis |
| SEQ ID NO: 20 | RNA for Abi27 | transcribed RNA | Lactococcus lactis |
| SEQ ID NO: 21 | AbiM | protein | Lactococcus lactis |
| SEQ ID NO: 22 | AbiW | protein | Lactococcus lactis |
| SEQ ID NO: 23 | AbiX | protein | Lactococcus lactis |
| SEQ ID NO: 24 | AbiZA | protein | Lactococcus lactis |
| SEQ ID NO: 25 | AbiYi | protein | Lactococcus lactis |
| SEQ ID NO: 26 | AbiYii | protein | Lactococcus lactis |
| SEQ ID NO: 27 | Abi27a | protein | Lactococcus lactis |
| SEQ ID NO: 28 | Abi27b | protein | Lactococcus lactis |
| SEQ ID NO: 29 | pNZ44_F | other DNA | synthetic construct |
| SEQ ID NO: 30 | pNZ44_R | other DNA | synthetic construct |
| SEQ ID NO: 31 | pPTPi_F | other DNA | synthetic construct |
| SEQ ID NO: 32 | pPTPi_R | other DNA | synthetic construct |
| SEQ ID NO: 33 | pLL69075_B49_FW | other DNA | synthetic construct |
| SEQ ID NO: 34 | pLL69075_B49_RV | other DNA | synthetic construct |
| SEQ ID NO: 35 | NZ9000::pJP005_FW | other DNA | synthetic construct |
| SEQ ID NO: 36 | NZ9000:pJP005_RV | other DNA | synthetic construct |
| SEQ ID NO: 37 | MG1614_FW | other DNA | synthetic construct |
| SEQ ID NO: 38 | MG1614_RV | other DNA | synthetic construct |
| SEQ ID NO: 39 | LL64983_FW | other DNA | synthetic construct |
| SEQ ID NO: 40 | LL64983_RV | other DNA | synthetic construct |
| SEQ ID NO: 41 | pLL79476_B52_FW | other DNA | synthetic construct |
| SEQ ID NO: 42 | pLL79476_B52_RV | other DNA | synthetic construct |
| SEQ ID NO: 43 | pLL81569_D29_FW | other DNA | synthetic construct |
| SEQ ID NO: 44 | pLL81569_D29_RV | other DNA | synthetic construct |
| SEQ ID NO: 45 | pLL66563_D54_FW | other DNA | synthetic construct |
| SEQ ID NO: 46 | pLL66563_D54_RV | other DNA | synthetic construct |
| SEQ ID NO: 47 | pLL66563_D53_FW | other DNA | synthetic construct |
| SEQ ID NO: 48 | pLL66563_D53_RV | other DNA | synthetic construct |
| SEQ ID NO: 49 | pLL75953_C24_FW | other DNA | synthetic construct |
| SEQ ID NO: 50 | pLL75953_C24_RV | other DNA | synthetic construct |
| SEQ ID NO: 51 | pLL75953_C25_FW | other DNA | synthetic construct |
| SEQ ID NO: 52 | pLL75953_C25_RV | other DNA | synthetic construct |
| SEQ ID NO: 53 | pLL75843_E85_FW | other DNA | synthetic construct |
| SEQ ID NO: 54 | pLL75843_E85_RV | other DNA | synthetic construct |
| SEQ ID NO: 55 | abi28a (LL75972_pC26) | other DNA | Lactococcus lactis |
| SEQ ID NO: 56 | abi28b (LL75972_pC25) | other DNA | Lactococcus lactis |
| SEQ ID NO: 57 | abi29a (LL75843_pB62) | other DNA | Lactococcus lactis |
| SEQ ID NO: 58 | abi29b (LL75843_pB61) | other DNA | Lactococcus lactis |
| SEQ ID NO: 59 | abi29c (LL75843_pB60) | other DNA | Lactococcus lactis |
| SEQ ID NO: 60 | abi30 (LL79472_pA59) | other DNA | Lactococcus lactis |
| SEQ ID NO: 61 | abi31 (LL66563_pC18) | other DNA | Lactococcus lactis |
| SEQ ID NO: 62 | abi32a (LL66563_pD51) | other DNA | Lactococcus lactis |
| SEQ ID NO: 63 | abi32b (LL66563_pD50) | other DNA | Lactococcus lactis |
| SEQ ID NO: 64 | abi32c (LL66563_pD49) | other DNA | Lactococcus lactis |
| SEQ ID NO: 65 | abi33a (pLL56542_pE17) | other DNA | Lactococcus lactis |
| SEQ ID NO: 66 | abi33b (pLL56542_pE16) | other DNA | Lactococcus lactis |
| SEQ ID NO: 67 | abi34 (LLA22_pB040) | other DNA | Lactococcus lactis |
| SEQ ID NO: 68 | Abi28a | protein | Lactococcus lactis |
| SEQ ID NO: 69 | Abi28b | protein | Lactococcus lactis |
| SEQ ID NO: 70 | Abi29a | protein | Lactococcus lactis |
| SEQ ID NO: 71 | Abi29b | protein | Lactococcus lactis |
| SEQ ID NO: 72 | Abi29c | protein | Lactococcus lactis |
| SEQ ID NO: 73 | Abi30 | protein | Lactococcus lactis |
| SEQ ID NO: 74 | Abi31 | protein | Lactococcus lactis |
| SEQ ID NO: 75 | Abi32a | protein | Lactococcus lactis |
| SEQ ID NO: 76 | Abi32b | protein | Lactococcus lactis |
| SEQ ID NO: 77 | Abi32c | protein | Lactococcus lactis |
| SEQ ID NO: 78 | Abi33a | protein | Lactococcus lactis |
| SEQ ID NO: 79 | Abi33b | protein | Lactococcus lactis |
| SEQ ID NO: 80 | Abi34 | protein | Lactococcus lactis |
| SEQ ID NO: 81 | LL75972_pC26_FW | other DNA | Synthetic construct |
| SEQ ID NO: 82 | LL75972_pC25_RV | other DNA | Synthetic construct |
| SEQ ID NO: 83 | LL75843_pB62_FW | other DNA | Synthetic construct |
| SEQ ID NO: 84 | LL75843_pB60_RV | other DNA | Synthetic construct |
| SEQ ID NO: 85 | LL79472_pA59_FW | other DNA | Synthetic construct |
| SEQ ID NO: 86 | LL79472_pA59_RV | other DNA | Synthetic construct |
| SEQ ID NO: 87 | LL66563_pC18_FW | other DNA | Synthetic construct |
| SEQ ID NO: 88 | LL66563_pC18_RV | other DNA | Synthetic construct |
| SEQ ID NO: 89 | LL66563_pD51_FW | other DNA | Synthetic construct |
| SEQ ID NO: 90 | LL66563_pD49_RV | other DNA | Synthetic construct |
| SEQ ID NO: 91 | LLA22_pB0040_FW | other DNA | Synthetic construct |
| SEQ ID NO: 92 | LLA22_pB0040_RV | other DNA | Synthetic construct |

### DETAILED DESCRIPTION

### General Definitions

Unless defined otherwise or clearly indicated by context, all technical and scientific terms used herein have the same meaning as commonly understood by one of ordinary skill in the art.

By the term “Abi system” is herein preferably understood an abortive infection system. The Abi systems and their mechanisms are diverse. They can affect different critical stages of the phage cycle, such as DNA replication, transcription, protein production, phage packaging and cell lysis.

A “nucleic acid construct” or “nucleic acid vector” is herein understood to mean a man-made nucleic acid molecule, resulting preferably from the use of recombinant DNA technology. The term “nucleic acid construct” therefore does not include naturally occurring nucleic acid molecules although a nucleic acid construct may comprise (parts of) naturally occurring nucleic acid molecules. The terms “expression vector” or “expression construct” refer to nucleotide sequences that are capable of effecting expression of a gene in host cells or host organisms compatible with such sequences. These expression vectors typically include at least suitable transcription regulatory sequences and optionally, 3′ transcription termination signals. Additional factors necessary or helpful in effecting expression may also be present, such as expression enhancer elements. The expression vector will be introduced into a suitable host cell and be able to effect expression of the coding sequence in an in vitro cell culture of the host cell. The expression vector will be suitable for replication in the host cell or organism of the invention.

As used herein, the term “promoter” or “transcription regulatory sequence” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” promoter is a promoter that is physiologically or developmentally regulated, e.g. by the application of a chemical inducer. An inducible promoter may also be present but not induced.

The term “selectable marker” is a term familiar to one of ordinary skill in the art and is used herein to describe any genetic entity which, when expressed, can be used to select for a cell or cells containing the selectable marker. The term “reporter” may be used interchangeably with marker, although it is mainly used to refer to visible markers, such as green fluorescent protein (GFP). Selectable markers may be dominant or recessive or bidirectional.

As used herein, the term “operably linked” refers to a linkage of polynucleotide elements in a functional relationship. A nucleic acid is “operably linked” when it is placed into a functional relationship with another nucleic acid sequence. For instance, a transcription regulatory sequence is operably linked to a coding sequence if it affects the transcription of the coding sequence. Operably linked means that the DNA sequences being linked are typically contiguous and, where necessary to join two protein encoding regions, contiguous and in reading frame.

The term “gene” means a DNA fragment comprising a region (transcribed region), which is transcribed into an RNA molecule (e.g. an mRNA) in a cell, operably linked to suitable regulatory regions (e.g. a promoter). A gene will usually comprise several operably linked fragments, such as a promoter, a 5′ leader sequence, a coding region, exons, introns and a 3′-nontranslated sequence (3′-end) e.g. comprising a polyadenylation- and/or transcription termination site.

“Expression of a gene” refers to the process wherein a DNA region which is operably linked to appropriate regulatory regions, particularly a promoter, is transcribed into an RNA, which is biologically active, i.e. which is capable of being translated into a biologically active protein or peptide.

The term “homologous” when used to indicate the relation between a given (recombinant) nucleic acid or polypeptide molecule and a given host organism or host cell, is understood to mean that in nature the nucleic acid or polypeptide molecule is produced by a host cell or organisms of the same species, preferably of the same variety or strain. If homologous to a host cell, a nucleic acid sequence encoding a polypeptide will typically (but not necessarily) be operably linked to another (heterologous) promoter sequence and, if applicable, another (heterologous) secretory signal sequence and/or terminator sequence than in its natural environment. It is understood that the regulatory sequences, signal sequences, terminator sequences, etc. may also be homologous to the host cell. In this context, the use of only “homologous” sequence elements allows the construction of “self-cloned” genetically modified organisms (GMO's) (self-cloning is defined herein as in European Directive 98/81/EC Annex II). When used to indicate the relatedness of two nucleic acid sequences the term “homologous” means that one single-stranded nucleic acid sequence may hybridize to a complementary single-stranded nucleic acid sequence. The degree of hybridization may depend on a number of factors including the amount of identity between the sequences and the hybridization conditions such as temperature and salt concentration as discussed earlier herein.

The terms “heterologous” and “exogenous” when used with respect to a nucleic acid (DNA or RNA) or protein refers to a nucleic acid or protein that does not occur naturally as part of the organism, cell, genome or DNA or RNA sequence in which it is present, or that is found in a cell or location or locations in the genome or DNA or RNA sequence that differ from that in which it is found in nature. Heterologous and exogenous nucleic acids or proteins are not endogenous to the cell into which it is introduced, but have been obtained from another cell or synthetically or recombinantly produced. Generally, though not necessarily, such nucleic acids encode proteins, i.e. exogenous proteins, that are not normally produced by the cell in which the DNA is transcribed or expressed. Similarly exogenous RNA encodes for proteins not normally expressed in the cell in which the exogenous RNA is present. Heterologous/exogenous nucleic acids and proteins may also be referred to as foreign nucleic acids or proteins. Any nucleic acid or protein that one of skill in the art would recognize as foreign to the cell in which it is expressed is herein encompassed by the term heterologous or exogenous nucleic acid or protein. The terms heterologous and exogenous also apply to non-natural combinations of nucleic acid or amino acid sequences, i.e. combinations where at least two of the combined sequences are foreign with respect to each other. The terms heterologous and exogenous specifically also apply to non-naturally occurring modified versions of otherwise endogenous nucleic acids or proteins.

As used herein the term “milk substrate” (also referred to as “milk base”) is the starting material, or starting substrate, for the fermentation process to provide a fermented milk product. It includes whole milk, skim milk, fat-free milk, low fat milk, full fat milk, lactose-free or lactose-reduced milk (produced by hydrolyzing the lactose by lactase enzyme to glucose and galactose, or by other methods such as nanofiltration, electro dialysis, ion exchange chromatography and centrifugation technology), concentrated milk or dry milk. It can also include synthetic milk. It can also include plant-based milk or non-dairy milk. As used herein, “fat-free milk” is non-fat or skim milk product. Low-fat milk is typically defined as milk that contains from about 1% to about 2% fat. Full fat milk often contains 2% fat or more.

As used herein, the term “milk” encompasses milks from mammals and plant sources or mixtures thereof. It can also comprise a “synthetic milk” (see further below). Preferably, the milk is from a mammal source. Mammals sources of milk include, but are not limited to cow, sheep, goat, buffalo, camel, llama, mare and deer. In an embodiment, the milk is from a mammal selected from the group consisting of cow, sheep, goat, buffalo, camel, llama, mare and deer, and combinations thereof. Plant sources of milk include, but are not limited to, milk extracted from soy bean, pea, peanut, barley, rice, oat, quinoa, almond, cashew, coconut, hazelnut, hemp, sesame seed and sunflower seed. Soy bean milk is preferred. In addition, the term “milk” refers to not only whole milk, but also skim milk or any liquid component derived thereof.

A synthetic milk is defined herein as a liquid that has been (re) constituted from proteins produced by methods such as fermentation, such as precision fermentation. Synthetic dairy companies (like e.g., Vivici, and Perfect Day) are producing milk proteins using a process known as precision fermentation. It involves genetically engineering yeast or other microorganisms using (synthetic) DNA to produce a specific milk protein. Such milk proteins, produced by the process of precision fermentation, are subsequently blended with other (milk) proteins (either isolated from animal milk, or produced by precision fermentation), minerals, vitamins, lipids (fat) and other constituents normally present in animal milk, yielding a synthetic milk.

As used herein, the term “fermented milk product” or “acidified dairy product” refers to products which are obtained by the multiplication of lactic acid bacteria in a milk base leading to a milk coagulum. The milk preparation used as raw material for the fermentation may be skimmed or non-skimmed milk, optionally concentrated or in the form of powder. Furthermore, this milk preparation may have been subjected to a thermal processing operation which is at least as efficient as pasteurization. The particular characteristics of the various fermented dairy products depend upon various factors, such as the composition of milk base, the incubation temperature, the lactic acid flora and/or non-lactic acid flora. Thus, fermented dairy products manufactured herein include, various types of regular yoghurt, low fat yoghurt, non fat yoghurt, kefir, dahi, ymer, buttermilk, butterfat, sour cream and sour whipped cream as well as fresh cheeses and quark and twarog. A fermented dairy product may further comprise other cheeses such as soft cheeses or cream cheeses or semi-hard cheeses or hard cheeses or ripened cheeses. These types may comprise familiar cheese types such as cheddar or Gouda or Edam or Maasdam or feta or Camembert or Brie or cottage cheese or Manchego. A preferred fermented milk product is a cheese, preferably a cheese as described here above.

The term “fermentation” or “fermentation process” is herein broadly defined in accordance with its common definition as used in industry as any (large-scale) microbial process occurring in the presence or absence of oxygen, comprising the cultivation of at least one microorganism whereby preferably the microorganism produces a useful product at the expense of consuming one or more organic substrates. The term “fermentation” is herein thus has a much broader definition than the more strict scientific definition wherein it is defined as being limited a microbial process wherein the microorganism extracts energy from carbohydrates in the absence of oxygen. Likewise, the term “fermentation product” is herein broadly defined as any useful product produced in a (large-scale) microbial process occurring in the presence or absence of oxygen.

The term “starter composition” or “starter culture” as used herein refers to a culture of one or more food-grade micro-organisms, in particular lactic acid bacteria, which are responsible for the acidification of the milk base. Starter cultures may be fresh (liquid), frozen or freeze-dried. Freeze dried cultures need to be regenerated before use. For the production of a fermented dairy product, the starter is usually added in an amount from 0.01 to 3%, preferably from 0.01 and 0.02% by weight of the total amount of milk base.

As used herein, the terms “host cell”, “cell” and “strain” are used interchangeably. As used herein, the term “lactic acid bacteria” (LAB) or “lactic bacteria” refers to food-grade bacteria producing lactic acid as the major metabolic end-product of carbohydrate fermentation. These bacteria are related by their common metabolic and physiological characteristics and are usually Gram positive, low-GC, acid tolerant, non-sporulating, non-respiring, rod-shaped bacilli, or cocci. During the fermentation stage, the consumption of lactose by these bacteria causes the formation of lactic acid, reducing the pH and leading to the formation of a protein coagulum. These bacteria are thus responsible for the acidification of milk and for the texture of the dairy product. As used herein, the term “lactic acid bacteria” or “lactic bacteria” encompasses, but is not limited to, bacteria belonging to the genus of Lactobacillus spp., Bifidobacterium spp., Streptococcus spp., Lactococcus spp., such as Lactobacillus delbruekii subsp. bulgaricus, Streptococcus thermophilus, Lactobacillus lactis, Bifidobacterium animalis, Lactococcus lactis, Lactococcus cremoris, Lactocaseibacillus casei (formerly named Lactobacillus casei), Lactiplantibacillus plantarum (formerly named Lactobacillus plantarum), Lactobacillus helveticus, Lactobacillus acidophilus and Bifidobacterium breve.

“Sequence identity” is herein defined as a relationship between two or more amino acid (peptide, polypeptide, or protein) sequences or two or more nucleic acid (nucleotide, polynucleotide) sequences, as determined by comparing the sequences. In the art, “identity” also means the degree of sequence relatedness between amino acid or nucleotide sequences, as the case may be, as determined by the match between strings of such sequences. “Similarity” between two amino acid sequences is determined by comparing the amino acid sequence and its conserved amino acid substitutes of one peptide or polypeptide to the sequence of a second peptide or polypeptide. In a preferred embodiment, identity or similarity is calculated over the whole SEQ ID NO as identified herein. “Identity” and “similarity” can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Preferred methods to determine identity are designed to give the largest match between the sequences tested. Methods to determine identity and similarity are codified in publicly available computer programs. Preferred computer program methods to determine identity and similarity between two sequences include e.g. the GCG program package (Devereux, J., et al., Nucleic Acids Research 12 (1): 387 (1984)), BestFit, BLASTP, BLASTN, and FASTA (Altschul, S. F. et al., J. Mol. Biol. 215:403-410 (1990). The BLAST X program is publicly available from NCBI and other sources (BLAST Manual, Altschul, S., et al., NCBI NLM NIH Bethesda, MD 20894; Altschul, S., et al., J. Mol. Biol. 215:403-410 (1990). The well-known Smith Waterman algorithm may also be used to determine identity.

Preferred parameters for polypeptide sequence comparison include the following: Algorithm: Needleman and Wunsch, J. Mol. Biol. 48:443-453 (1970); Comparison matrix: BLOSUM62 from Hentikoff and Hentikoff, Proc. Natl. Acad. Sci. USA. 89:10915-10919 (1992); Gap Penalty: 12; and Gap Length Penalty: 4. A program useful with these parameters is publicly available as the “Ogap” program from Genetics Computer Group, located in Madison, WI. The aforementioned parameters are the default parameters for amino acid comparisons (along with no penalty for end gaps).

Preferred parameters for nucleic acid comparison include the following: Algorithm: Needleman and Wunsch, J. Mol. Biol. 48:443-453 (1970); Comparison matrix: matches=+10, mismatch=0; Gap Penalty: 50; Gap Length Penalty: 3. Available as the Gap program from Genetics Computer Group, located in Madison, Wis. Given above are the default parameters for nucleic acid comparisons.

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to ser; Arg to lys; Asn to gln or his; Asp to glu; Cys to ser or ala; Gln to asn; Glu to asp; Gly to pro; His to asn or gln; Ile to leu or val; Leu to ile or val; Lys to arg; gln or glu; Met to leu or ile; Phe to met, leu or tyr; Ser to thr; Thr to ser; Trp to tyr; Tyr to trp or phe; and, Val to ile or leu.

A “nucleic acid molecule” or “polynucleotide” (the terms are used interchangeably herein) is represented by a nucleotide sequence. Sometimes in the field, the term “nucleotide sequence” or plainly “sequence” is used when an entity (polynucleotide) is intended. A “polypeptide” is represented by an amino acid sequence. A “polypeptide” as used herein refers to any peptide, oligopeptide, polypeptide, gene product, expression product, or protein. A polypeptide is comprised of consecutive amino acids. The term “polypeptide” encompasses naturally occurring and synthetic molecules.

The sequence information as provided herein should not be so narrowly construed as to require inclusion of erroneously identified bases. The skilled person is capable of identifying such erroneously identified bases and knows how to correct for such errors.

In this document and in its claims, the verb “to comprise” and its conjugations is used in its non-limiting sense to mean that items following the word are included, but items not specifically mentioned are not excluded. In addition the verb “to consist” may be replaced by “to consist essentially of” meaning that a product or a composition or a nucleic acid molecule or a peptide or polypeptide of a nucleic acid construct or vector or cell as defined herein may comprise additional component(s) than the ones specifically identified; said additional component(s) not altering the unique characteristic of the invention. In addition, reference to an element by the indefinite article “a” or “an” does not exclude the possibility that more than one of the elements is present, unless the context clearly requires that there be one and only one of the elements. The indefinite article “a” or “an” thus usually means “at least one”. The word “about” or “approximately” when used in association with a numerical value (e.g. about 10) preferably means that the value may be the given value (of 10) more or less 10% of the value.

Unless explicitly indicated otherwise, the various embodiments of the invention described herein can be cross-combined.

All patent and literature references cited in the present specification are hereby incorporated by reference in their entirety. Unless otherwise indicated each embodiment as described herein may be combined with another embodiment as described herein. The following examples are offered for illustrative purposes only, and are not intended to limit the scope of the present invention in any way.

### Nucleic Acids

As indicated above, the “nucleic acid construct” or “nucleic acid vector” is herein understood to mean a man-made nucleic acid molecule. In such embodiments, the term “nucleic acid construct” therefore does not include naturally occurring nucleic acid molecules although a nucleic acid construct may comprise (parts of) naturally occurring nucleic acid molecules. Preferably the “nucleic acid construct” or “nucleic acid vector” is the result from the use of recombinant DNA technology. However, in other embodiments, the “nucleic acid construct” may be a naturally occurring nucleic acid construct. Such construct may be isolated from a host cell and may be used in the methods as set forward herein, or such construct may be used within the host cell it occurs in a method according to the invention.

In a first aspect, there is provided for a nucleic acid construct comprising a first sequence (i.e. a first nucleotide sequence) that has at least 80% sequence identity to any one of SEQ ID NOs: 60, 1-8 and/or 55-59 and/or 61-67, or wherein the first sequence encodes a polypeptide with an amino acid sequence that has at least 80% sequence identity to the amino acid sequence as set forth in any one of SEQ ID NOS: 73, 21-28 and/or 68-72 and/or 74-80. In a further aspect, there is provided for a nucleic acid construct comprising a nucleotide sequence that has at least 80% sequence identity to SEQ ID NO: 9 or SEQ ID NO: 10. These sequences SEQ ID NO: 1-8 and 55-67, SEQ ID NO: 9, SEQ ID NO: 10 and sequences that encode a polypeptide with an amino acid sequence that has at least 80% sequence identity to the amino acid sequence as set forth in any one of SEQ ID NOs: 21-28 and/or 68-80, are referred to herein as sequences according to the invention. The nucleic acid construct is preferably an isolated nucleic acid construct.

In some preferred embodiments the nucleic acid construct, comprises, consists or essentially consists of said first sequence. In preferred embodiments the first sequence has at least 81, 82, 83, 84, 85, 86, 87, 88, 89, 90, 91, 92, 93, 94, 95, 96, 97, 98, 99, or 100% sequence identity with any one of SEQ ID NOS: 1-8 and/or 55-67, or the first sequence encodes a polypeptide with an amino acid sequence that has at least 81, 82, 83, 84, 85, 86, 87, 88, 89, 90, 91, 92, 93, 94, 95, 96, 97, 98, 99, or 100% sequence identity to the amino acid sequence as set forth in any one of SEQ ID NOs: 21-28 and/or 68-80. More preferably the first sequence has at least 85% sequence identity, even more preferably at least 90%, still more preferably at least 95%, still more preferably at least 98%, still more preferably at least 99%, most preferably at least 100% with any one of SEQ ID NOS: 1-8 and/or 55-67, or the first sequence encodes a polypeptide with an amino acid sequence that has at least 85% sequence identity, even more preferably at least 90%, still more preferably at least 95%, still more preferably at least 98%, still more preferably at least 99%, most preferably at least 100% sequence identity to the amino acid sequence as set forth in any one of SEQ ID NOs: 21-28 and/or 68-80. In some embodiments, the first sequence has at least one mutation, more preferably a silent mutation.

In a preferred embodiment, the invention provides a nucleic acid construct comprising a first sequence that:
has at least 80%, preferably at least 90%, more preferably, in order of preference, 95%, 99%, 99.90% or 99.99%, and most preferably 100% sequence identity with SEQ ID NO: 1; or
has at least 80%, preferably at least 90%, more preferably, in order of preference, 95%, 99%, 99.90% or 99.99%, and most preferably 100% sequence identity with SEQ ID NO: 2; or
has at least 80%, preferably at least 90%, more preferably, in order of preference, 95%, 99%, 99.90% or 99.99%, and most preferably 100% sequence identity with SEQ ID NO: 3; or
has at least 80%, preferably at least 90%, more preferably, in order of preference, 95%, 99%, 99.90% or 99.99%, and most preferably 100% sequence identity with SEQ ID NO: 4; or
has at least 80%, preferably at least 90%, more preferably, in order of preference, 95%, 99%, 99.90% or 99.99%, and most preferably 100% sequence identity with SEQ ID NO: 5; or
has at least 80%, preferably at least 90%, more preferably, in order of preference, 95%, 99%, 99.90% or 99.99%, and most preferably 100% sequence identity with SEQ ID NO: 6; or
has at least 80%, preferably at least 90%, more preferably, in order of preference, 95%, 99%, 99.90% or 99.99%, and most preferably 100% sequence identity with SEQ ID NO: 7; or
has at least 80%, preferably at least 90%, more preferably, in order of preference, 95%, 99%, 99.90% or 99.99%, and most preferably 100% sequence identity with SEQ ID NO: 8; or
has at least 80%, preferably at least 90%, more preferably, in order of preference, 95%, 99%, 99.90% or 99.99%, and most preferably 100% sequence identity with SEQ ID NO: 55; or
has at least 80%, preferably at least 90%, more preferably, in order of preference, 95%, 99%, 99.90% or 99.99%, and most preferably 100% sequence identity with SEQ ID NO: 56; or
has at least 80%, preferably at least 90%, more preferably, in order of preference, 95%, 99%, 99.90% or 99.99%, and most preferably 100% sequence identity with SEQ ID NO: 57, or
has at least 80%, preferably at least 90%, more preferably, in order of preference, 95%, 99%, 99.90% or 99.99%, and most preferably 100% sequence identity with SEQ ID NO: 58; or
has at least 80%, preferably at least 90%, more preferably, in order of preference, 95%, 99%, 99.90% or 99.99%, and most preferably 100% sequence identity with SEQ ID NO: 59; or
has at least 80%, preferably at least 90%, more preferably, in order of preference, 95%, 99%, 99.90% or 99.99%, and most preferably 100% sequence identity with SEQ ID NO: 60; or
has at least 80%, preferably at least 90%, more preferably, in order of preference, 95%, 99%, 99.90% or 99.99%, and most preferably 100% sequence identity with SEQ ID NO: 61; or
has at least 80%, preferably at least 90%, more preferably, in order of preference, 95%, 99%, 99.90% or 99.99%, and most preferably 100% sequence identity with SEQ ID NO: 62; or
has at least 80%, preferably at least 90%, more preferably, in order of preference, 95%, 99%, 99.90% or 99.99%, and most preferably 100% sequence identity with SEQ ID NO: 63; or
has at least 80%, preferably at least 90%, more preferably, in order of preference, 95%, 99%, 99.90% or 99.99%, and most preferably 100% sequence identity with SEQ ID NO: 64; or
has at least 80%, preferably at least 90%, more preferably, in order of preference, 95%, 99%, 99.90% or 99.99%, and most preferably 100% sequence identity with SEQ ID NO: 65; or
has at least 80%, preferably at least 90%, more preferably, in order of preference, 95%, 99%, 99.90% or 99.99%, and most preferably 100% sequence identity with SEQ ID NO: 66; or
has at least 80%, preferably at least 90%, more preferably, in order of preference, 95%, 99%, 99.90% or 99.99%, and most preferably 100% sequence identity with SEQ ID NO: 67; or
encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 21; or
encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 22; or
encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 23; or
encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 24; or
encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 25; or
encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 26; or
encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 27; or
encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 28; or
encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 68; or
encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 69; or
encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 70; or
encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 71; or
encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 72; or
encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 73; or
encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 74; or
encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 75; or
encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 76; or
encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 77; or
encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 78; or
encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 79; or
encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 80,
wherein preferably such first sequence is operably linked to a promotor; and/or
wherein preferably the construct encodes a polypeptide that confers improved phage resistance to a lactococcal host cell. The mentioned sequence may or may not have at least one mutation, more preferably a silent mutation. Preferably, the first nucleotide sequence is an isolated nucleotide sequence, meaning that it has been isolated from nature by man. Alternatively, the nucleotide sequence can be constructed synthetically.

Further preferences for this embodiment are as provided herein above and herein below.

In further preferred embodiments the nucleic acid construct, comprises, consists or essentially consists of a sequence, preferably an isolated or synthesized sequence, that has at least 81, 82, 83, 84, 85, 86, 87, 88, 89, 90, 91, 92, 93, 94, 95, 96, 97, 98, 99, or 100% sequence identity with SEQ ID NO: 9 or SEQ ID NO: 10. More preferably this sequence has at least 85% sequence identity, even more preferably at least 90%, still more preferably at least 95%, still more preferably at least 98%, still more preferably at least 99%, most preferably at least 100% with SEQ ID NO: 9 or SEQ ID NO: 10. In some embodiments, this sequence has at least one mutation, more preferably a silent mutation.

In a preferred embodiment, the invention provides a nucleic acid construct, wherein the nucleic acid construct, comprises consists or essentially consists of a sequence that has:
at least 80%, preferably at least 90%, more preferably, in order of preference, 95%, 99%, 99.90% or 99.99%, and most preferably 100% sequence identity with SEQ ID NO: 9; or
at least 80%, preferably at least 90%, more preferably, in order of preference, 95%, 99%, 99.90% or 99.99%, and most preferably 100% sequence identity with SEQ ID NO: 10,
wherein preferably the construct encodes a polypeptide that confers improved phage resistance to a lactococcal host cell.

As indicated above, preferred is a nucleic acid construct wherein the construct encodes a polypeptide that confers improved phage resistance to a lactococcal host cell. Transcription of any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO; 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66 and/or SEQ ID NO: 67 (herein also referred to as “SEQ ID NOS: 1-10 and/or 55-67”) may suitably lead to RNA. Suitably, transcription of the respective nucleotide sequences SEQ ID NOS: 1-10, each individually, may lead into the respective RNA sequence of SEQ ID NOs: 11-20. Expression of the nucleotide sequence SEQ ID NOs 1-10 and/or 55-67, respectively, into RNA may advantageously lead to production of proteins with amino acid sequence SEQ ID NOS: 21-28 and/or 68-80, respectively. This may advantageously confer phage resistance to the host cell. It is therefore preferred that the nucleotide sequences are transcriptionally active when comprised in a host cell. Accordingly, in preferred embodiments the first nucleotide sequence is operably linked to a promoter, preferably the native promoter. Suitable promoters can be selected by a skilled person. For exemplary SEQ ID NOs, the Examples section provides combinations of the SEQ ID NO with promoters, as also reflected in the sequence listing. In some embodiments the promoter is a constitutive promoter. In other embodiments the promoter is an inducible promoter, such as a nisin-inducible promoter.

Multiple sequences according to the invention can be combined. In the nucleic acid construct, the first sequence can be followed by a second sequence that has at least 80% sequence identity to any one of SEQ ID NOS: 1-8 and/or 55-67 or wherein the second sequence encodes a polypeptide with an amino acid sequence that has at least 80% sequence identity to the amino acid sequence as set forth in any one of SEQ ID NOs: 21-28 and/or 68-80, wherein preferably the first and the second sequence have at least 80% sequence identity with SEQ ID NOs: 5 and 6, 7 and 8, 55 and 56, or 65 and 66, respectively, or wherein the first and second sequence preferably encode a polypeptide with an amino acid sequence that has at least 80% sequence identity to the amino acid sequence as set forth in SEQ ID NO:s 25 and 26, 27 and 28, 68 and 69, or 78 and 79, respectively.

In the nucleic acid construct, the first and second sequences can be followed by a third sequence that has at least 80% sequence identity to any one of SEQ ID NOs: 1-8 and/or 55-67, or wherein the third sequence encodes a polypeptide with an amino acid sequence that has at least 80% sequence identity to the amino acid sequence as set forth in any one of SEQ ID NOs: 21-28 and/or 68-80, wherein preferably the first, second and third sequence have at least 80% sequence identity with SEQ ID NOS: 57, 58, and 59 and 62, 63 and 64, respectively, or wherein the first second and third sequence encode a polypeptide with an amino acid sequence that has at least 80% sequence identity to the amino acid sequence as set forth in SEQ ID NOs: 70, 71 and 72, or 75, 76 and 77, respectively.

In some embodiments, 2 sequences are present in the construct. In other embodiments, 3, 4, 5, 6, 7, or 8 sequences are present. Preferred combinations are SEQ ID NOs: 5 and 6 (which are combined in SEQ ID NO: 9), or SEQ ID NOs: 7 and 8 (which are combined in SEQ ID NO: 10,) SEQ ID NOs: 55 and 56, SEQ ID NOs: 65 and 66, SEQ ID NO: 57, 58 and 59, and SEQ ID NOs: 62, 63 and 64. Further preferred combinations are a pair of sequences that encode a polypeptide with an amino acid sequence that has at least 80% sequence identity to the amino acid sequence as set forth in SEQ ID NOs: 25 and 26, 27 and 28, 68 and 69, or 78 and 79, respectively. Further preferred combinations are a set of three sequences that encode a polypeptide with an amino acid sequence that has at least 80% sequence identity to the amino acid sequence as set forth in SEQ ID NOs: 70, 71 and 72, or 75, 76 and 77, respectively. The multiple sequences can each be individually linked to a promoter, or can be under the control of a single promoter, for instance in a single operon. Combinations can also be made, where for instance any one of SEQ ID NOs: 1-4 and/or 60, 61 and 67 are each operably linked to an individual promoter, while SEQ ID NOs: 5 and 6, SEQ ID NOs: 7 and 8, SEQ ID NOS: 55 and 56, SEQ ID NOs: 65 and 66, SEQ ID NOs: 57, 58 and 59, and SEQ ID NOs: 62, 63 and 64 are present on or within a single operon (such as SEQ ID NOs: 9 and 10).

An overview of the respective nucleotide sequences, RNA sequences and protein sequences is provided below in Table 2. In addition, an indication of preferred DNA primer pairs is provided, although the person skilled in the art is perfectly capable of designing primers for amplication. When used, each nucleotide sequence, that is each “ORF”, is preferably operationally linked to a promoter as explained above.

TABLE 2
Overview of the respective nucleotide sequences,
RNA sequences and protein sequences

| | | | | | Abi detection |
|---|---|---|---|---|---|
| | | ORF | RNA | | DNA primer |
| Abi | | SEQ ID | SEQ ID | Protein | pair |
| system | Strain/Plasmid/ORF | NO | NO | SEQ ID NO | SEQ ID NO |
| AbiM | LL79476_pB52 | 1 | 11 | 21 | 41 |
| | | | | | 42 |
| AbiW | LL81569_pD29 | 2 | 12 | 22 | 43 |
| | | | | | 44 |
| AbiX | LL69075_pB49 | 3 | 13 | 23 | 33 |
| | | | | | 34 |
| AbiZA | LL75843_pE85 | 4 | 14 | 24 | 53 |
| | | | | | 54 |
| AbiYi | LL66563_pD54 | 5 | 15 | 25 | 45 |
| | | | | | 46 |
| AbiYii | LL66563_pD53 | 6 | 16 | 26 | 47 |
| | | | | | 48 |
| AbiY | LL66563_pD54_53 | 9 | 19 | | 45 |
| | | | | | 48 |
| Abi27a | LL75953_pC24 | 7 | 17 | 27 | 49 |
| | | | | | 50 |
| Abi27b | LL75953_pC25 | 8 | 18 | 28 | 51 |
| | | | | | 52 |
| Abi27 | LL75953_pC24_25 | 10 | 20 | | 49 |
| | | | | | 52 |
| Abi28a | LL75972_pC26 | 55 | | 68 | 81-82 |
| Abi28b | LL75972_pC25 | 56 | | 69 | 81-82 |
| Abi29a | LL75843_pB62 | 57 | | 70 | 83-84 |
| Abi29b | LL75843_pB61 | 58 | | 71 | 83-84 |
| Abi29c | LL75843_pB60 | 59 | | 72 | 83-84 |
| Abi30 | LL79472_pA59 | 60 | | 73 | 85-86 |
| Abi31 | LL66563_pC18 | 61 | | 74 | 85-86 |
| Abi32a | LL66563_pD51 | 62 | | 75 | 89-90 |
| Abi32b | LL66563_pD50 | 63 | | 76 | 89-90 |
| Abi32c | LL66563_pD49 | 64 | | 77 | 89-90 |
| Abi33a | pLL56542_pE17 | 65 | | 78 | |
| Abi33b | pLL56542_pE16 | 66 | | 79 | |
| Abi34 | LLA22_pB040 | 67 | | 80 | 91-92 |

In one preferred embodiment, the nucleic acid construct, respectively the plasmid, comprises a first sequence and a second sequence, wherein the first sequence has preferably at least 80%, more preferably at least 90%, still more preferably at least 95%, yet more preferably equal to or more than 99%, still even more preferably equal to or more than 99.90%, yet even more preferably equal to or more than 99.99% and most preferably 100% sequence identity with SEQ ID NO: 5 and the second sequence has preferably at least 80%, more preferably at least 90%, still more preferably at least 95%, yet more preferably equal to or more than 99%, still even more preferably equal to or more than 99.90%, yet even more preferably equal to or more than 99.99% and most preferably 100% sequence identity with SEQ ID NO: 6.

In another preferred embodiment, the nucleic acid construct, respectively the plasmid, comprises a first sequence and a second sequence, wherein the first sequence has preferably at least 80%, more preferably at least 90%, still more preferably at least 95%, yet more preferably equal to or more than 99%, still even more preferably equal to or more than 99.90%, yet even more preferably equal to or more than 99.99% and most preferably 100% sequence identity with SEQ ID NO: 7 and the second sequence has preferably at least 80%, more preferably at least 90%, still more preferably at least 95%, yet more preferably equal to or more than 99%, still even more preferably equal to or more than 99.90%, yet even more preferably equal to or more than 99.99% and most preferably 100% sequence identity with SEQ ID NO: 8.

The nucleic acid constructs can conveniently be comprised in a plasmid. Plasmids can be linear or circular, where circular plasmids are preferred. Accordingly, provided is a plasmid comprising the nucleic acid construct as described above, wherein the plasmid is preferably a conjugative plasmid, more preferably a lactococcal conjugative plasmid.

A skilled person can select a useful plasmid. For instance, for cloning with an intent to multiply the sequences according to the invention, a plasmid should generally consist of a backbone and an insert, wherein the insert can be the sequence according to the invention. Such backbones then comprise the required regulatory elements and selectable markers. Suitable backbones in this case can be any one of the plasmids pNZ44 (McGrath et al., 2001), pPTPi (O'Driscoll et al., 2004), pPEPi, PPTPL-skicos (Mahony et al., 2008), pJP005 (Van Pijkeren & Britton, 2012) respectively.

Plasmids can also be naturally occurring plasmids or derivatives thereof, such as any one of pLL79476B (AbiM), pLL81569D (AbiW), pLL69075B (AbiX), pLL66563D (AbiY), pLL75953C (Abi27), pLL75843E (AbiZA), pLL79472A (Abi30), pLL66563C (Abi31), pLLA22B (Abi34), respectively. A pLL66563D (AbiY) and/or pLL75953C (Abi27) and/or pLL75972C (Abi28) and/or pLL56542E (Abi33) comprising a 2-component system is especially preferred. A pLL75843B (Abi29) and/or pLL66563D (Abi32) comprising a 3-component-system is especially preferred.

In a preferred embodiment, the invention provides a vector, preferably a plasmid, wherein the vector, preferably the plasmid is preferably a conjugative plasmid, more preferably a lactococcal conjugative plasmid,
wherein the plasmid comprises a nucleic acid construct,
(i) wherein the nucleic acid construct comprises a first sequence that:
   has at least 80%, preferably at least 90%, more preferably, in order of preference, 95%, 99%, 99.90% or 99.99%, and most preferably 100% sequence identity with SEQ ID NO: 1; or
   has at least 80%, preferably at least 90%, more preferably, in order of preference, 95%, 99%, 99.90% or 99.99%, and most preferably 100% sequence identity with SEQ ID NO: 2; or
   has at least 80%, preferably at least 90%, more preferably, in order of preference, 95%, 99%, 99.90% or 99.99%, and most preferably 100% sequence identity with SEQ ID NO: 3; or
   has at least 80%, preferably at least 90%, more preferably, in order of preference, 95%, 99%, 99.90% or 99.99%, and most preferably 100% sequence identity with SEQ ID NO: 4; or
   has at least 80%, preferably at least 90%, more preferably, in order of preference, 95%, 99%, 99.90% or 99.99%, and most preferably 100% sequence identity with SEQ ID NO: 5; or
   has at least 80%, preferably at least 90%, more preferably, in order of preference, 95%, 99%, 99.90% or 99.99%, and most preferably 100% sequence identity with SEQ ID NO: 6; or
   has at least 80%, preferably at least 90%, more preferably, in order of preference, 95%, 99%, 99.90% or 99.99%, and most preferably 100% sequence identity with SEQ ID NO: 7; or
   has at least 80%, preferably at least 90%, more preferably, in order of preference, 95%, 99%, 99.90% or 99.99%, and most preferably 100% sequence identity with SEQ ID NO: 8; or
   has at least 80%, preferably at least 90%, more preferably, in order of preference, 95%, 99%, 99.90% or 99.99%, and most preferably 100% sequence identity with SEQ ID NO: 55; or
   has at least 80%, preferably at least 90%, more preferably, in order of preference, 95%, 99%, 99.90% or 99.99%, and most preferably 100% sequence identity with SEQ ID NO: 56; or
   has at least 80%, preferably at least 90%, more preferably, in order of preference, 95%, 99%, 99.90% or 99.99%, and most preferably 100% sequence identity with SEQ ID NO: 57; or
   has at least 80%, preferably at least 90%, more preferably, in order of preference, 95%, 99%, 99.90% or 99.99%, and most preferably 100% sequence identity with SEQ ID NO: 58;
   has at least 80%, preferably at least 90%, more preferably, in order of preference, 95%, 99%, 99.90% or 99.99%, and most preferably 100% sequence identity with SEQ ID NO: 59; or
   has at least 80%, preferably at least 90%, more preferably, in order of preference, 95%, 99%, 99.90% or 99.99%, and most preferably 100% sequence identity with SEQ ID NO: 60; or
   has at least 80%, preferably at least 90%, more preferably, in order of preference, 95%, 99%, 99.90% or 99.99%, and most preferably 100% sequence identity with SEQ ID NO: 61; or
   has at least 80%, preferably at least 90%, more preferably, in order of preference, 95%, 99%, 99.90% or 99.99%, and most preferably 100% sequence identity with SEQ ID NO: 62; or
   has at least 80%, preferably at least 90%, more preferably, in order of preference, 95%, 99%, 99.90% or 99.99%, and most preferably 100% sequence identity with SEQ ID NO: 63; or
   has at least 80%, preferably at least 90%, more preferably, in order of preference, 95%, 99%, 99.90% or 99.99%, and most preferably 100% sequence identity with SEQ ID NO: 64; or
   has at least 80%, preferably at least 90%, more preferably, in order of preference, 95%, 99%, 99.90% or 99.99%, and most preferably 100% sequence identity with SEQ ID NO: 65; or
   has at least 80%, preferably at least 90%, more preferably, in order of preference, 95%, 99%, 99.90% or 99.99%, and most preferably 100% sequence identity with SEQ ID NO: 66; or
   has at least 80%, preferably at least 90%, more preferably, in order of preference, 95%, 99%, 99.90% or 99.99%, and most preferably 100% sequence identity with SEQ ID NO: 67; or
   encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 21; or
   encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 22; or
   encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 23; or
   encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 24; or
   encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 25; or
   encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 26; or
   encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 27; or
   encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 28; or
   encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 68; or
   encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 69; or
   encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 70; or
   encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 71; or
   encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 72; or
   encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 73; or
   encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 74; or
   encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 75; or
   encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 76; or
   encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 77; or
   encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 78; or
   encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 79; or
   encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 80,
wherein preferably such first sequence is operably linked to a promotor; and/or wherein preferably the construct encodes a polypeptide that confers improved phage resistance to a lactococcal host cell;
and/or
(ii) wherein the nucleic acid construct, comprises consists or essentially consists of a sequence that has:
   at least 80%, preferably at least 90%, more preferably, in order of preference, 95%, 99%, 99.90% or 99.99%, and most preferably 100% sequence identity with SEQ ID NO: 9; or
   at least 80%, preferably at least 90%, more preferably, in order of preference, 95%, 99%, 99.90% or 99.99%, and most preferably 100% sequence identity with SEQ ID NO: 10,
wherein preferably such first sequence is operably linked to a promotor; and/or
wherein preferably the construct encodes a polypeptide that confers improved phage resistance to a lactococcal host cell;
and/or
(iii) wherein the nucleic acid construct comprises, consists or essentially consists of two sequences that have:
   at least 80%, preferably at least 90%, more preferably, in order of preference, 95%, 99%, 99.90% or 99.99%, and most preferably 100% sequence identity with SEQ ID NO: 55 and at least 80%, preferably at least 90%, more preferably, in order of preference, 95%, 99%, 99.90% or 99.99%, and most preferably 100% sequence identity with SEQ ID NO: 56; or
   at least 80%, preferably at least 90%, more preferably, in order of preference, 95%, 99%, 99.90% or 99.99%, and most preferably 100% sequence identity with SEQ ID NO: 65 and at least 80%, preferably at least 90%, more preferably, in order of preference, 95%, 99%, 99.90% or 99.99%, and most preferably 100% sequence identity with SEQ ID NO: 66;
wherein preferably such sequences are operably linked to a promotor; and/or
wherein preferably the construct encodes a polypeptide that confers improved phage resistance to a lactococcal host cell; and/or
(iv) wherein the nucleic acid construct comprises, consists or essentially consists of three sequences that have:
   at least 80%, preferably at least 90%, more preferably, in order of preference, 95%, 99%, 99.90% or 99.99%, and most preferably 100% sequence identity with SEQ ID NO: 57, at least 80%, preferably at least 90%, more preferably, in order of preference, 95%, 99%, 99.90% or 99.99%, and most preferably 100% sequence identity with SEQ ID NO: 58, and at least 80%, preferably at least 90%, more preferably, in order of preference, 95%, 99%, 99.90% or 99.99%, and most preferably 100% sequence identity with SEQ ID NO: 59; or
   at least 80%, preferably at least 90%, more preferably, in order of preference, 95%, 99%, 99.90% or 99.99%, and most preferably 100% sequence identity with SEQ ID NO: 62, at least 80%, preferably at least 90%, more preferably, in order of preference, 95%, 99%, 99.90% or 99.99%, and most preferably 100% sequence identity with SEQ ID NO: 63, and at least 80%, preferably at least 90%, more preferably, in order of preference, 95%, 99%, 99.90% or 99.99%, and most preferably 100% sequence identity with SEQ ID NO: 64; and/or
(v) wherein the nucleic acid construct comprises, consists or essentially consists of two sequences that encode:
   a polypeptide with an amino acid sequence that has at least 80%, preferably at least 90%, more preferably, in order of preference, 95%, 99%, 99.90% or 99.99%, and most preferably 100% sequence identity to the amino sequence as set forth in SEQ ID NO: 25 and at least 80%, preferably at least 90%, more preferably, in order of preference, 95%, 99%, 99.90% or 99.99%, and most preferably 100% sequence identity to the amino sequence as set forth in SEQ ID NO: 26; or
   a polypeptide with an amino acid sequence that has at least 80%, preferably at least 90%, more preferably, in order of preference, 95%, 99%, 99.90% or 99.99%, and most preferably 100% sequence identity to the amino sequence as set forth in SEQ ID NO: 27 and at least 80%, preferably at least 90%, more preferably, in order of preference, 95%, 99%, 99.90% or 99.99%, and most preferably 100% sequence identity to the amino sequence as set forth in SEQ ID NO: 28; or
   a polypeptide with an amino acid sequence that has at least 80%, preferably at least 90%, more preferably, in order of preference, 95%, 99%, 99.90% or 99.99%, and most preferably 100% sequence identity to the amino sequence as set forth in SEQ ID NO: 68 and at least 80%, preferably at least 90%, more preferably, in order of preference, 95%, 99%, 99.90% or 99.99%, and most preferably 100% sequence identity to the amino sequence as set forth in SEQ ID NO: 69; or
   a polypeptide with an amino acid sequence that has at least 80%, preferably at least 90%, more preferably, in order of preference, 95%, 99%, 99.90% or 99.99%, and most preferably 100% sequence identity to the amino sequence as set forth in SEQ ID NO: 78 and at least 80%, preferably at least 90%, more preferably, in order of preference, 95%, 99%, 99.90% or 99.99%, and most preferably 100% sequence identity to the amino sequence as set forth in SEQ ID NO: 79;
wherein preferably such sequences are operably linked to a promotor; and/or
wherein preferably the construct encodes a polypeptide that confers improved phage resistance to a lactococcal host cell; and/or
(vi) wherein the nucleic acid construct comprises, consists or essentially consists of three sequences that encode:
   a polypeptide with an amino acid sequence that has at least 80%, preferably at least 90%, more preferably, in order of preference, 95%, 99%, 99.90% or 99.99%, and most preferably 100% sequence identity to the amino sequence as set forth in SEQ ID NO: 70, at least 80%, preferably at least 90%, more preferably, in order of preference, 95%, 99%, 99.90% or 99.99%, and most preferably 100% sequence identity to the amino sequence as set forth in SEQ ID NO: 71, and at least 80%, preferably at least 90%, more preferably, in order of preference, 95%, 99%, 99.90% or 99.99%, and most preferably 100% sequence identity to the amino sequence as set forth in SEQ ID NO: 72; or
   a polypeptide with an amino acid sequence that has at least 80%, preferably at least 90%, more preferably, in order of preference, 95%, 99%, 99.90% or 99.99%, and most preferably 100% sequence identity to the amino sequence as set forth in SEQ ID NO: 75, at least 80%, preferably at least 90%, more preferably, in order of preference, 95%, 99%, 99.90% or 99.99%, and most preferably 100% sequence identity to the amino sequence as set forth in SEQ ID NO: 76, and at least 80%, preferably at least 90%, more preferably, in order of preference, 95%, 99%, 99.90% or 99.99%, and most preferably 100% sequence identity to the amino sequence as set forth in SEQ ID NO: 77;
wherein preferably such sequences are operably linked to a promotor; and/or
wherein preferably the construct encodes a polypeptide that confers improved phage resistance to a lactococcal host cell.

Further preferences for such a plasmid are as described herein above and herein below.

The plasmids may conveniently comprise a first sequence, a second sequence and third sequence as explained above. In the context of this invention a derivative of a naturally occurring plasmid preferably has one mutation, more preferably a silent mutation. Naturally occurring plasmids are convenient for use in generating Lactococcus strains with improved phage resistance. These allow their anti-phage activities to be transferred by conjugation to generate robust starter strains with enhanced phage-resistance properties without yielding genetically modified organisms. It follows that preferred plasmids are conjugative plasmids, which preferably comprise conjugation clusters. As is known in the art, a plasmid can be considered conjugative when it encodes one or several relaxases, a VirB4, a T4CP, and a number of additional mating pair formation (MPF) proteins, as is described by Ortiz-Charnaco et al. (2021). The required number of MPF can vary according to the MPF type: two proteins for MPFFA and MPFFATA and three for the other MPF types (B, C, F, G, I, and T) (Guglielmini et al., 2014). Thereby, a conjugative plasmid preferably comprises a cluster of tra genes.

Within the scope of the invention, the conjugative plasmid may also be a mobilizable plasmid. This type of plasmid may be present in a strain without the conjugative plasmid and may be considered non-conjugative in such strain. However, the mobilizable plasmid may be (co-)mobilised upon the introduction of a conjugative plasmid to said strain which can subsequently utilized as donor strain of the genetic trait, in this case (a) mobilizable plasmid(s) bearing Abi systems described in present invention. Ortiz-Charnaco et al. (2021) states that such plasmids may comprise an origin of transfer (oriT) sequence and at least one mobilization gene: mobA, mobD (encoding nickases), mobB and mobC (encoding proteins that are thought to form a relaxosome with an associated nickase, either mobA or mobD) (O'Brien et al., 2015; Kelleher et al., 2019). Plasmids with highly similar oriT sites may lack mob or tra genes but, when a conjugative plasmid is present together with a non-conjugative plasmid in the same donor strain, the relaxase from the conjugative plasmid may recognize the oriT sequence within the non-conjugative plasmid, promoting transfer of either or both plasmids to a recipient cell (Francia et al., 2004).

The desired traits conferred by sequences according to the invention can be transferred among strains using conjugative plasmids, thus conferring benefits on the host (Mills et al., 2006). Conjugation is a non-GMO, horizontal gene transfer (HGT) mechanism that involves natural transfer of genetic material from a donor to a recipient cell via a conjugative apparatus through direct cell-to-cell contact (Kohler et al., 2019). Conjugation is regarded a food-grade process (Mills et al., 2006). The desired traits conferred by sequences according to the invention may also be transferred among strains by natural competence. Natural competence is a non-GMO, horizontal gene transfer (HGT) mechanism that involves natural transfer of genetic material (which may comprise plasmid DNA or linear DNA) through uptake of said genetic material in the environment by a DNA uptake system (encoded by com genes). Successful plasmid DNA transfer to a recipient Lactococcus strain has been described by Mulder et al. (2017).

Preferred plasmids may comprise a restriction modification system. Such systems are known in the art and provide defence against foreign DNA, such as bacteriophage DNA. Preferred plasmids may also comprise a further Abi system in addition to an Abi system according to the invention.

### Polypeptides

The nucleic acid sequences according to the invention can suitably encode polypeptides. These polypeptides can suitably confer improved phage resistance to host cells comprising the polypeptides. The invention thus provides these polypeptides. The polypeptides preferably comprise or consist of an amino acid sequence that has at least 80% sequence identity, preferably at least 90% sequence identity, more preferably at least 95% sequence identity, even more preferably at least 99% sequence identity, and most preferably 100% sequence identity to any one of amino acid sequences SEQ ID NOS: 21-28 and/or 68-80. These polypeptides are referred to herein as polypeptides according to the invention.

In a further aspect, the invention therefore also provides a polypeptide that comprises or consists of an amino acid sequence that has at least 80% sequence identity, preferably at least 90% sequence identity, more preferably at least 95% sequence identity, even more preferably at least 99% sequence identity, and most preferably 100% sequence identity, to any one of amino acid sequences SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 68, SEQ NO: 69, SEQ ID NO; 70, SEQ ID NO: 71, SEQ ID NO: 72, SEQ ID NO: 73, SEQ ID NO; 74, SEQ ID NO: 75, SEQ ID NO: 76, SEQ ID NO: 77, SEQ ID NO: 78, SEQ ID NO: 79 and/or SEQ ID NO: 80 (herein also referred to as “SEQ ID NOs: 21-28 and/or 68-80”). The polypeptide is preferably an isolated polypeptide. In preferred embodiments the polypeptide consists or essentially consists of said sequence. In preferred embodiments the amino acid sequence has at least 81, 82, 83, 84, 85, 86, 87, 88, 89, 90, 91, 92, 93, 94, 95, 96, 97, 98, 99, or 100% sequence identity with any one of SEQ ID NOs: 21-28 and 68-80. More preferably the amino acid sequence has at least 85% sequence identity, even more preferably at least 90%, still more preferably at least 95%, still more preferably at least 98%, still more preferably at least 99%, most preferably at least 100% sequence identity, to any one of amino acid sequences SEQ ID NOs: 21-28 and/or 68-80. In some embodiments, the sequence of the polypeptide may have at least one mutation, more preferably a silent mutation, or it is extended by one amino acid, or by one or more amino acids, or it is truncated by one amino acid, or it is truncated by one or more amino acids. A preferred polypeptide comprises a transmembrane domain.

Also provided are nucleic acid constructs that encode a polypeptide according to the invention. Such nucleic acid constructs further preferably have features as described elsewhere herein.

The polypeptide may advantageously confer improved phage resistance, preferably via abortive infection of phages in a Lactococcus cell. Preferably, the resistance is to a phage from the phage group Skunavirus, P335, P087, or Ceduovirus. Examples of Skunavirus are sk1, 712, p2, JJ50, 66901, 62601, bIL66, bIL70, P008, P113G, and 340. Examples of p335 are TP901-1, LC3, Dub35A, and 63301. An example of Ceduovirus is c2. An example of P087 is P087. Preferably, resistance is to Skunavirus or p335 or Ceduovirus. Most preferably resistance is to Skunavirus. Preferably, phage resistance is present when efficiency of plaquing (EOP) is reduced or when plaque size is reduced. Plaque size is preferably reduced by at least about 30%, more preferably by at least about 50%. EOP is preferably reduced to 10⁻⁴, more preferably 10⁻⁵, still more preferably 10⁻⁶, still more preferably 10⁻⁸, even more preferably 10⁻⁹; most preferably, complete resistance is conferred whereby no plaques are observed after the introduction of the nucleic acid construct while the respective control is still susceptible to the phage. EOP is preferably determined by dividing the titre (pfu/ml) of a phage propagated on a strain comprising the sequence to be assessed by that of the same phage propagated on a control strain. A control strain preferably differs from the strain to be assessed only in the presence of the sequence to be assessed, or of a nucleic acid construct comprising that sequence. For instance, the control strain can comprise an empty vector where the strain to be assessed comprises that vector carrying the relevant nucleic acid sequence. Suitable methods are described in the examples.

In preferred embodiments, phage resistance is resistance to Skunavirus. In other preferred embodiments, phage resistance is resistance to Skunavirus and at least one P335 phage, preferably TP901-1. In other preferred embodiments, phage resistance is resistance to Skunavirus and Ceduovirus. In highly preferred embodiments, phage resistance is resistance to Skunavirus and at least one P335 phage, preferably TP901-1, and Ceduovirus.

### Cells and Cultures

Novel and robust starter strains comprising the above-mentioned features may be created by classical means or by modern biotechnology. Advantageously, phage resistance in Lactococcus can be obtained via the new phage resistance mechanisms, such as abortive infection mechanisms, which can advantageously be encoded by any one of SEQ ID NOs: 1-10 and/or 55-67, as detailed above, and preferably having an amino acid sequence as set forth in SEQ ID NO: 21-28 and/or 68-80.

Phage resistance is a trait of a bacterial cell. Host cells comprising the nucleic acid constructs according to the invention are therefore preferably bacterial cells that are preferably capable of expressing or multiplying the nucleic acid sequences according to the invention. Host cells can also be for multiplication of the sequence according to the invention, in which case the host cell can be any suitable microbial cell, such as E. coli.

In an aspect is provided for a host cell preferably comprising the nucleic acid construct according to the invention, or the plasmid according to the invention. Preferably, such host cell according to the invention is an isolated host cell. The host cell is preferably a Lactococcus, such as Lactococcus chungangensis, Lactococcus formosensis, Lactococcus fujiensis, Lactococcus garvieae, L. garvieae subsp. garvieae, L. garvieae subsp. bovis, Lactococcus hircilactis, Lactococcus lactis, L. cremoris, L. lactis subsp. hordniae, L. lactis subsp. lactis, L. lactis subsp. tructae, Lactococcus laudensis, Lactococcus nasutitermitis, Lactococcus piscium, Lactococcus plantarum, Lactococcus raffinolactis, or Lactococcus taiwanensis. Preferred Lactococcus are Lactococcus lactis, and subspecies such as Lactococcus lactis subsp. diacetylactis and Lactococcus cremoris (formerly assigned as two subspecies of Lactococcus lactis).

In preferred embodiments the host cell comprises the nucleic acid construct according to the invention, wherein the nucleic acid construct is heterologous to the host cell. In more preferred embodiments the host cell comprises the plasmid according to the invention, wherein the plasmid is heterologous to the host cell.

In preferred embodiments the host cell is capable of expressing the polypeptide according to the invention. In preferred embodiments the nucleic acid construct according to the invention confers phage resistance to the host cell. In preferred embodiments the polypeptide according to the invention confers phage resistance to the host cell.

The host cell can be obtained by conjugation, transformation, mutagenesis, or genome editing, and subsequent selection for improved phage resistance. Selection for improved phage resistance can be performed by exposing a cultured host cell to the phage. The examples provide suitable conditions for this. Means for conjugation, transformation, mutagenesis, or genome editing are widely known and examples are provided later herein.

A preferred host cell is obtained by conjugation, resulting in a transconjugated host cell. The host cell can be obtained through transconjugation of plasmid or chromosomal DNA and the transconjugated host cell preferably expresses one of the nucleic acid constructs according to the invention.

The person skilled in the art will comprehend that a host cell may comprise more than one nucleic acid construct and/or nucleotide sequence according to the invention, such as two, three, four, five, six, seven, eight, nine, ten, eleven, twelve or thirteen nucleic acid constructs according to the invention. This means that the host cell according to the invention may comprise two or more different Abi systems.

In an especially preferred embodiment, the invention thus provides a Lactococcus cell, preferably Lactococcus lactis cell or a Lactococcus cremoris cell, wherein the Lactococcus cell comprises a nucleic acid construct, more preferably a plasmid comprising a nucleic acid construct, wherein the nucleic acid construct is heterologous to the cell and/or the plasmid is heterologous to the cell and
(i) wherein the nucleic acid construct comprises a first sequence that:
   has at least 80%, preferably at least 90%, more preferably, in order of preference, 95%, 99%, 99.90% or 99.99%, and most preferably 100% sequence identity with SEQ ID NO: 1; or
   has at least 80%, preferably at least 90%, more preferably, in order of preference, 95%, 99%, 99.90% or 99.99%, and most preferably 100% sequence identity with SEQ ID NO: 2; or
   has at least 80%, preferably at least 90%, more preferably, in order of preference, 95%, 99%, 99.90% or 99.99%, and most preferably 100% sequence identity with SEQ ID NO: 3; or
   has at least 80%, preferably at least 90%, more preferably, in order of preference, 95%, 99%, 99.90% or 99.99%, and most preferably 100% sequence identity with SEQ ID NO: 4; or
   has at least 80%, preferably at least 90%, more preferably, in order of preference, 95%, 99%, 99.90% or 99.99%, and most preferably 100% sequence identity with SEQ ID NO: 5; or
   has at least 80%, preferably at least 90%, more preferably, in order of preference, 95%, 99%, 99.90% or 99.99%, and most preferably 100% sequence identity with SEQ ID NO: 6; or
   has at least 80%, preferably at least 90%, more preferably, in order of preference, 95%, 99%, 99.90% or 99.99%, and most preferably 100% sequence identity with SEQ ID NO: 7; or
   has at least 80%, preferably at least 90%, more preferably, in order of preference, 95%, 99%, 99.90% or 99.99%, and most preferably 100% sequence identity with SEQ ID NO: 8, or
   has at least 80%, preferably at least 90%, more preferably, in order of preference, 95%, 99%, 99.90% or 99.99%, and most preferably 100% sequence identity with SEQ ID NO: 55, or
   has at least 80%, preferably at least 90%, more preferably, in order of preference, 95%, 99%, 99.90% or 99.99%, and most preferably 100% sequence identity with SEQ ID NO: 56, or
   has at least 80%, preferably at least 90%, more preferably, in order of preference, 95%, 99%, 99.90% or 99.99%, and most preferably 100% sequence identity with SEQ ID NO: 57, or
   has at least 80%, preferably at least 90%, more preferably, in order of preference, 95%, 99%, 99.90% or 99.99%, and most preferably 100% sequence identity with SEQ ID NO: 58, or
   has at least 80%, preferably at least 90%, more preferably, in order of preference, 95%, 99%, 99.90% or 99.99%, and most preferably 100% sequence identity with SEQ ID NO: 59, or
   has at least 80%, preferably at least 90%, more preferably, in order of preference, 95%, 99%, 99.90% or 99.99%, and most preferably 100% sequence identity with SEQ ID NO: 60, or
   has at least 80%, preferably at least 90%, more preferably, in order of preference, 95%, 99%, 99.90% or 99.99%, and most preferably 100% sequence identity with SEQ ID NO: 61, or
   has at least 80%, preferably at least 90%, more preferably, in order of preference, 95%, 99%, 99.90% or 99.99%, and most preferably 100% sequence identity with SEQ ID NO: 62, or
   has at least 80%, preferably at least 90%, more preferably, in order of preference, 95%, 99%, 99.90% or 99.99%, and most preferably 100% sequence identity with SEQ ID NO: 63, or
   has at least 80%, preferably at least 90%, more preferably, in order of preference, 95%, 99%, 99.90% or 99.99%, and most preferably 100% sequence identity with SEQ ID NO: 64, or
   has at least 80%, preferably at least 90%, more preferably, in order of preference, 95%, 99%, 99.90% or 99.99%, and most preferably 100% sequence identity with SEQ ID NO: 65, or
   has at least 80%, preferably at least 90%, more preferably, in order of preference, 95%, 99%, 99.90% or 99.99%, and most preferably 100% sequence identity with SEQ ID NO: 66, or
   has at least 80%, preferably at least 90%, more preferably, in order of preference, 95%, 99%, 99.90% or 99.99%, and most preferably 100% sequence identity with SEQ ID NO: 67, or
   encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 21; or
   encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 22; or
   encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 23; or
   encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 24; or
   encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 25; or
   encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 26; or
   encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 27; or
   encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 28; or
   encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 68; or
   encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 69; or
   encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 70; or
   encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 71; or
   encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 72; or
   encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 73; or
   encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 74; or
   encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 75; or
   encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 76; or
   encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 77; or
   encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 78; or
   encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 79; or
   encodes a polypeptide with an amino acid sequence that has at least 80%, preferably at least 90%, more preferably, in order of preference, 95%, 99%, 99.90% or 99.99%, and most preferably 100% sequence identity with SEQ ID NO: 80,
wherein preferably such first sequence is operably linked to a promotor; and/or wherein preferably the construct encodes a polypeptide that confers improved phage resistance to a lactococcal host cell;
and/or
(ii) wherein the nucleic acid construct comprises, consists or essentially consists of a sequence that has:
   at least 80%, preferably at least 90%, more preferably, in order of preference, 95%, 99%, 99.90% or 99.99%, and most preferably 100% sequence identity with SEQ ID NO: 9; or
   at least 80%, preferably at least 90%, more preferably, in order of preference, 95%, 99%, 99.90% or 99.99%, and most preferably 100% sequence identity with SEQ ID NO: 10,
wherein preferably the construct encodes a polypeptide that confers improved phage resistance to a lactococcal host cell;
and/or
(iii) wherein the nucleic acid construct comprises, consists or essentially consists of two sequences that have:
   at least 80%, preferably at least 90%, more preferably, in order of preference, 95%, 99%, 99.90% or 99.99%, and most preferably 100% sequence identity with SEQ ID NO: 55 and at least 80%, preferably at least 90%, more preferably, in order of preference, 95%, 99%, 99.90% or 99.99%, and most preferably 100% sequence identity with SEQ ID NO: 56; or
   at least 80%, preferably at least 90%, more preferably, in order of preference, 95%, 99%, 99.90% or 99.99%, and most preferably 100% sequence identity with SEQ ID NO: 65 and at least 80%, preferably at least 90%, more preferably, in order of preference, 95%, 99%, 99.90% or 99.99%, and most preferably 100% sequence identity with SEQ ID NO: 66;
wherein preferably such sequences are operably linked to a promotor; and/or
wherein preferably the construct encodes a polypeptide that confers improved phage resistance to a lactococcal host cell; and/or
(iv) wherein the nucleic acid construct comprises, consists or essentially consists of three sequences that have:
   at least 80%, preferably at least 90%, more preferably, in order of preference, 95%, 99%, 99.90% or 99.99%, and most preferably 100% sequence identity with SEQ ID NO: 57, at least 80%, preferably at least 90%, more preferably, in order of preference, 95%, 99%, 99.90% or 99.99%, and most preferably 100% sequence identity with SEQ ID NO: 58, and at least 80%, preferably at least 90%, more preferably, in order of preference, 95%, 99%, 99.90% or 99.99%, and most preferably 100% sequence identity with SEQ ID NO: 59; or
   at least 80%, preferably at least 90%, more preferably, in order of preference, 95%, 99%, 99.90% or 99.99%, and most preferably 100% sequence identity with SEQ ID NO: 62, at least 80%, preferably at least 90%, more preferably, in order of preference, 95%, 99%, 99.90% or 99.99%, and most preferably 100% sequence identity with SEQ ID NO: 63, and at least 80%, preferably at least 90%, more preferably, in order of preference, 95%, 99%, 99.90% or 99.99%, and most preferably 100% sequence identity with SEQ ID NO: 64; and/or
(v) wherein the nucleic acid construct comprises, consists or essentially consists of two sequences that encode:
   a polypeptide with an amino acid sequence that has at least 80%, preferably at least 90%, more preferably, in order of preference, 95%, 99%, 99.90% or 99.99%, and most preferably 100% sequence identity to the amino sequence as set forth in SEQ ID NO: 25 and at least 80%, preferably at least 90%, more preferably, in order of preference, 95%, 99%, 99.90% or 99.99%, and most preferably 100% sequence identity to the amino sequence as set forth in SEQ ID NO: 26; or
   a polypeptide with an amino acid sequence that has at least 80%, preferably at least 90%, more preferably, in order of preference, 95%, 99%, 99.90% or 99.99%, and most preferably 100% sequence identity to the amino sequence as set forth in SEQ ID NO: 27 and at least 80%, preferably at least 90%, more preferably, in order of preference, 95%, 99%, 99.90% or 99.99%, and most preferably 100% sequence identity to the amino sequence as set forth in SEQ ID NO: 28; or—a polypeptide with an amino acid sequence that has at least 80%, preferably at least 90%, more preferably, in order of preference, 95%, 99%, 99.90% or 99.99%, and most preferably 100% sequence identity to the amino sequence as set forth in SEQ ID NO: 68 and at least 80%, preferably at least 90%, more preferably, in order of preference, 95%, 99%, 99.90% or 99.99%, and most preferably 100% sequence identity to the amino sequence as set forth in SEQ ID NO: 69; or
   a polypeptide with an amino acid sequence that has at least 80%, preferably at least 90%, more preferably, in order of preference, 95%, 99%, 99.90% or 99.99%, and most preferably 100% sequence identity to the amino sequence as set forth in SEQ ID NO: 78 and at least 80%, preferably at least 90%, more preferably, in order of preference, 95%, 99%, 99.90% or 99.99%, and most preferably 100% sequence identity to the amino sequence as set forth in SEQ ID NO: 79;
wherein preferably such sequences are operably linked to a promotor; and/or
wherein preferably the construct encodes a polypeptide that confers improved phage resistance to a lactococcal host cell; and/or
(vi) wherein the nucleic acid construct comprises, consists or essentially consists of three sequences that encode:
   a polypeptide with an amino acid sequence that has at least 80%, preferably at least 90%, more preferably, in order of preference, 95%, 99%, 99.90% or 99.99%, and most preferably 100% sequence identity to the amino sequence as set forth in SEQ ID NO: 70, at least 80%, preferably at least 90%, more preferably, in order of preference, 95%, 99%, 99.90% or 99.99%, and most preferably 100% sequence identity to the amino sequence as set forth in SEQ ID NO: 71, and at least 80%, preferably at least 90%, more preferably, in order of preference, 95%, 99%, 99.90% or 99.99%, and most preferably 100% sequence identity to the amino sequence as set forth in SEQ ID NO: 72; or
   a polypeptide with an amino acid sequence that has at least 80%, preferably at least 90%, more preferably, in order of preference, 95%, 99%, 99.90% or 99.99%, and most preferably 100% sequence identity to the amino sequence as set forth in SEQ ID NO: 75, at least 80%, preferably at least 90%, more preferably, in order of preference, 95%, 99%, 99.90% or 99.99%, and most preferably 100% sequence identity to the amino sequence as set forth in SEQ ID NO: 76, and at least 80%, preferably at least 90%, more preferably, in order of preference, 95%, 99%, 99.90% or 99.99%, and most preferably 100% sequence identity to the amino sequence as set forth in SEQ ID NO: 77;
wherein preferably such sequences are operably linked to a promotor; and/or
wherein preferably the construct encodes a polypeptide that confers improved phage resistance to a lactococcal host cell.

In an especially preferred embodiment, the invention thus provides a Lactococcus cell, preferably Lactococcus lactis cell or a Lactococcus cremoris cell, wherein the Lactococcus cell comprises a nucleic acid sequence, more preferably a plasmid comprising a nucleic acid sequence, wherein the nucleic nucleic sequence is heterologous to the cell and/or the plasmid is heterologous to the cell and
(i) wherein the nucleic acid sequence comprises a first sequence that:
   has at least 80%, preferably at least 90%, more preferably, in order of preference, 95%, 99%, 99.90% or 99.99%, and most preferably 100% sequence identity with SEQ ID NO: 1; or
   has at least 80%, preferably at least 90%, more preferably, in order of preference, 95%, 99%, 99.90% or 99.99%, and most preferably 100% sequence identity with SEQ ID NO: 2; or
   has at least 80%, preferably at least 90%, more preferably, in order of preference, 95%, 99%, 99.90% or 99.99%, and most preferably 100% sequence identity with SEQ ID NO: 3; or
   has at least 80%, preferably at least 90%, more preferably, in order of preference, 95%, 99%, 99.90% or 99.99%, and most preferably 100% sequence identity with SEQ ID NO: 4; or
   has at least 80%, preferably at least 90%, more preferably, in order of preference, 95%, 99%, 99.90% or 99.99%, and most preferably 100% sequence identity with SEQ ID NO: 5; or
   has at least 80%, preferably at least 90%, more preferably, in order of preference, 95%, 99%, 99.90% or 99.99%, and most preferably 100% sequence identity with SEQ ID NO: 6; or
   has at least 80%, preferably at least 90%, more preferably, in order of preference, 95%, 99%, 99.90% or 99.99%, and most preferably 100% sequence identity with SEQ ID NO: 7; or
   has at least 80%, preferably at least 90%, more preferably, in order of preference, 95%, 99%, 99.90% or 99.99%, and most preferably 100% sequence identity with SEQ ID NO: 8,
   has at least 80%, preferably at least 90%, more preferably, in order of preference, 95%, 99%, 99.90% or 99.99%, and most preferably 100% sequence identity with SEQ ID NO: 55,
   has at least 80%, preferably at least 90%, more preferably, in order of preference, 95%, 99%, 99.90% or 99.99%, and most preferably 100% sequence identity with SEQ ID NO: 56,
   has at least 80%, preferably at least 90%, more preferably, in order of preference, 95%, 99%, 99.90% or 99.99%, and most preferably 100% sequence identity with SEQ ID NO: 57,
   has at least 80%, preferably at least 90%, more preferably, in order of preference, 95%, 99%, 99.90% or 99.99%, and most preferably 100% sequence identity with SEQ ID NO: 58,
   has at least 80%, preferably at least 90%, more preferably, in order of preference, 95%, 99%, 99.90% or 99.99%, and most preferably 100% sequence identity with SEQ ID NO: 59,
   has at least 80%, preferably at least 90%, more preferably, in order of preference, 95%, 99%, 99.90% or 99.99%, and most preferably 100% sequence identity with SEQ ID NO: 60,
   has at least 80%, preferably at least 90%, more preferably, in order of preference, 95%, 99%, 99.90% or 99.99%, and most preferably 100% sequence identity with SEQ ID NO: 61,
   has at least 80%, preferably at least 90%, more preferably, in order of preference, 95%, 99%, 99.90% or 99.99%, and most preferably 100% sequence identity with SEQ ID NO: 62,
   has at least 80%, preferably at least 90%, more preferably, in order of preference, 95%, 99%, 99.90% or 99.99%, and most preferably 100% sequence identity with SEQ ID NO: 63,
   has at least 80%, preferably at least 90%, more preferably, in order of preference, 95%, 99%, 99.90% or 99.99%, and most preferably 100% sequence identity with SEQ ID NO: 64,
   has at least 80%, preferably at least 90%, more preferably, in order of preference, 95%, 99%, 99.90% or 99.99%, and most preferably 100% sequence identity with SEQ ID NO: 65,
   has at least 80%, preferably at least 90%, more preferably, in order of preference, 95%, 99%, 99.90% or 99.99%, and most preferably 100% sequence identity with SEQ ID NO: 66,
   has at least 80%, preferably at least 90%, more preferably, in order of preference, 95%, 99%, 99.90% or 99.99%, and most preferably 100% sequence identity with SEQ ID NO: 67,
wherein preferably such first sequence is operably linked to a promotor; and/or wherein preferably the nucleic acid sequence encodes a polypeptide that confers improved phage resistance to a lactococcal host cell;
and/or
(ii) wherein the nucleic acid sequence consists or essentially consists of a sequence that has:
   at least 80%, preferably at least 90%, more preferably, in order of preference, 95%, 99%, 99.90% or 99.99%, and most preferably 100% sequence identity with SEQ ID NO: 9; or
   at least 80%, preferably at least 90%, more preferably, in order of preference, 95%, 99%, 99.90% or 99.99%, and most preferably 100% sequence identity with SEQ ID NO: 10,
wherein preferably the nucleic acid sequence encodes a polypeptide that confers improved phage resistance to a lactococcal host cell;
and/or
(iii) wherein the nucleic acid construct comprises, consists or essentially consists of two sequences that have:
   at least 80%, preferably at least 90%, more preferably, in order of preference, 95%, 99%, 99.90% or 99.99%, and most preferably 100% sequence identity with SEQ ID NO: 55 and at least 80%, preferably at least 90%, more preferably, in order of preference, 95%, 99%, 99.90% or 99.99%, and most preferably 100% sequence identity with SEQ ID NO: 56; or
   at least 80%, preferably at least 90%, more preferably, in order of preference, 95%, 99%, 99.90% or 99.99%, and most preferably 100% sequence identity with SEQ ID NO: 65 and at least 80%, preferably at least 90%, more preferably, in order of preference, 95%, 99%, 99.90% or 99.99%, and most preferably 100% sequence identity with SEQ ID NO: 66;
wherein preferably such sequences are operably linked to a promotor; and/or
wherein preferably the construct encodes a polypeptide that confers improved phage resistance to a lactococcal host cell; and/or
(iv) wherein the nucleic acid construct comprises, consists or essentially consists of three sequences that have:
   at least 80%, preferably at least 90%, more preferably, in order of preference, 95%, 99%, 99.90% or 99.99%, and most preferably 100% sequence identity with SEQ ID NO: 57, at least 80%, preferably at least 90%, more preferably, in order of preference, 95%, 99%, 99.90% or 99.99%, and most preferably 100% sequence identity with SEQ ID NO: 58, and at least 80%, preferably at least 90%, more preferably, in order of preference, 95%, 99%, 99.90% or 99.99%, and most preferably 100% sequence identity with SEQ ID NO: 59; or
   at least 80%, preferably at least 90%, more preferably, in order of preference, 95%, 99%, 99.90% or 99.99%, and most preferably 100% sequence identity with SEQ ID NO: 62, at least 80%, preferably at least 90%, more preferably, in order of preference, 95%, 99%, 99.90% or 99.99%, and most preferably 100% sequence identity with SEQ ID NO: 63, and at least 80%, preferably at least 90%, more preferably, in order of preference, 95%, 99%, 99.90% or 99.99%, and most preferably 100% sequence identity with SEQ ID NO: 64; and/or
(v) wherein the nucleic acid construct comprises, consists or essentially consists of two sequences that encode:
   a polypeptide with an amino acid sequence that has at least 80%, preferably at least 90%, more preferably, in order of preference, 95%, 99%, 99.90% or 99.99%, and most preferably 100% sequence identity to the amino sequence as set forth in SEQ ID NO: 25 and at least 80%, preferably at least 90%, more preferably, in order of preference, 95%, 99%, 99.90% or 99.99%, and most preferably 100% sequence identity to the amino sequence as set forth in SEQ ID NO: 26; or
   a polypeptide with an amino acid sequence that has at least 80%, preferably at least 90%, more preferably, in order of preference, 95%, 99%, 99.90% or 99.99%, and most preferably 100% sequence identity to the amino sequence as set forth in SEQ ID NO: 27 and at least 80%, preferably at least 90%, more preferably, in order of preference, 95%, 99%, 99.90% or 99.99%, and most preferably 100% sequence identity to the amino sequence as set forth in SEQ ID NO: 28; or
   a polypeptide with an amino acid sequence that has at least 80%, preferably at least 90%, more preferably, in order of preference, 95%, 99%, 99.90% or 99.99%, and most preferably 100% sequence identity to the amino sequence as set forth in SEQ ID NO: 68 and at least 80%, preferably at least 90%, more preferably, in order of preference, 95%, 99%, 99.90% or 99.99%, and most preferably 100% sequence identity to the amino sequence as set forth in SEQ ID NO: 69; or
   a polypeptide with an amino acid sequence that has at least 80%, preferably at least 90%, more preferably, in order of preference, 95%, 99%, 99.90% or 99.99%, and most preferably 100% sequence identity to the amino sequence as set forth in SEQ ID NO: 78 and at least 80%, preferably at least 90%, more preferably, in order of preference, 95%, 99%, 99.90% or 99.99%, and most preferably 100% sequence identity to the amino sequence as set forth in SEQ ID NO: 79;
wherein preferably such sequences are operably linked to a promotor; and/or
wherein preferably the construct encodes a polypeptide that confers improved phage resistance to a lactococcal host cell; and/or
(vi) wherein the nucleic acid construct comprises, consists or essentially consists of three sequences that encode:
   a polypeptide with an amino acid sequence that has at least 80%, preferably at least 90%, more preferably, in order of preference, 95%, 99%, 99.90% or 99.99%, and most preferably 100% sequence identity to the amino sequence as set forth in SEQ ID NO: 70, at least 80%, preferably at least 90%, more preferably, in order of preference, 95%, 99%, 99.90% or 99.99%, and most preferably 100% sequence identity to the amino sequence as set forth in SEQ ID NO: 71, and at least 80%, preferably at least 90%, more preferably, in order of preference, 95%, 99%, 99.90% or 99.99%, and most preferably 100% sequence identity to the amino sequence as set forth in SEQ ID NO: 72; or
   a polypeptide with an amino acid sequence that has at least 80%, preferably at least 90%, more preferably, in order of preference, 95%, 99%, 99.90% or 99.99%, and most preferably 100% sequence identity to the amino sequence as set forth in SEQ ID NO: 75, at least 80%, preferably at least 90%, more preferably, in order of preference, 95%, 99%, 99.90% or 99.99%, and most preferably 100% sequence identity to the amino sequence as set forth in SEQ ID NO: 76, and at least 80%, preferably at least 90%, more preferably, in order of preference, 95%, 99%, 99.90% or 99.99%, and most preferably 100% sequence identity to the amino sequence as set forth in SEQ ID NO: 77;
wherein preferably such sequences are operably linked to a promotor; and/or
wherein preferably the construct encodes a polypeptide that confers improved phage resistance to a lactococcal host cell.

Also provided is a starter culture for the production of a fermented food product, comprising a host cell, preferably an isolated strain as described herein. The starter culture further preferably comprises an excipient such as a cryoprotectant, a lyoprotectant, an antioxidant, and/or a nutrient. The starter culture can be frozen, lyophilized, spray-dried, vacuum-dried, air dried, tray dried, or in liquid form. The starter culture preferably further comprises a further Lactococcus strain or a Lactobacillus strain or a Streptococcus strain, preferably a further Lactococcus strain or a Lactobacillus helveticus strain or a Streptococcus thermophilus strain, most preferably the starter culture further comprises a further Lactococcus strain, or a Lactobacillus helveticus strain, or a Streptococcus thermophilus strain, or both a Lactobacillus helveticus strain and a Streptococcus thermophilus strain. The starter culture is suitable for the production of a fermented milk product. The starter culture may be a composition or a kit of parts.

The sequences according to the invention can advantageously be used for improving the phage resistance of cells as discussed above. Provided herein is therefore also the use of a nucleic acid construct according to the invention or a plasmid according to the invention in the production of a Lactococcus strain with improved phage resistance. The use can entail conjugation of the plasmid or nucleic acid construct, transformation of the plasmid or nucleic acid construct, or genome editing to integrate the plasmid or nucleic acid construct in the genome of the strain. Suitable techniques are widely known. The strain is preferably isolated after its phage resistance has been improved. Improvement of resistance is preferably as described elsewhere herein.

The use described above is suitable for producing strains with improved phage resistance. Accordingly the invention provides a method for the production of a Lactococcus strain with improved phage resistance, the method comprising:
i) providing a recipient Lactococcus strain;
ii) contacting the recipient Lactococcus strain with a donor Lactococcus strain, wherein the donor strain comprises a conjugative plasmid that comprises a nucleotide sequence that has at least 80% sequence identity, preferably at least 90% sequence identity, more preferably at least 95% sequence identity, even more preferably at least 99% sequence identity, still even more preferably at least 99.90% sequence identity, yet even more preferably at least 99.99% sequence identity and most preferably 100% sequence identity, to any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66 and/or SEQ ID NO: 67 (herein also referred to as “SEQ ID NOs: 1-10 and/or 55-67”), or
a nucleotide sequence that encodes a polypeptide with an amino acid sequence that has at least 80% sequence identity to the amino acid sequence as set forth in any one of SEQ ID NO: 21, SEQ ID NO: 22, SEQ ID NO: 23, SEQ ID NO: 24, SEQ ID NO; 25, SEQ ID NO: 26, SEQ ID NO: 27, SEQ ID NO: 28, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77. SEQ ID NO: 78, SEQ ID NO: 79, and/or SEQ ID NO: 80,
to obtain a transconjugant;
iii) selecting for a transconjugant comprising the conjugative plasmid to identify a strain with improved phage resistance; and optionally
iv) isolating the strain with improved phage resistance.

Preferably the produced strain is an isolated strain. In step i) a recipient Lactococcus strain is provided. This can be any Lactococcus strain, preferably it is a strain that is suitable in processes for producing fermented food products.

In step ii) the recipient strain is transconjugated. It is contacted with a donor Lactococcus strain that comprises a conjugative plasmid that comprises a nucleotide sequence that has at least 80% sequence identity to any one of SEQ ID NOS: 1-10 and/or 55-67, or that comprises a nucleotide sequence that encodes a polypeptide with an amino acid sequence that has at least 80% sequence identity to any one of SEQ ID NOS: 21-28 and/or 68-80. The plasmid can be a conjugative plasmid, but it is also possible to use a plasmid that has been mobilized from the donor to the recipient strain due to the presence of a conjugative plasmid in the donor cell (as for example illustrated by Ortiz Charneco et al. (2021). The latter is also considered covered by the claims and above embodiment. Preferably, the plasmid is a conjugative plasmid.

Preferably the plasmid is a conjugative plasmid as described elsewhere herein. It is preferred that the recipient strain and the donor strain are not identical. More particularly, it is preferred that the recipient strain, at the time of its provision in step i), does not comprise the conjugative plasmid. After transconjugation (conjugative transfer), the recipient strain has received the conjugative plasmid and is thus a transconjugant.

In step iii) the transconjugant is selected. This can be done using any means known in the art. For instance, a single selection strategy for the recipient strain can be performed, for instance using agar plates supplemented with either streptomycin, chloramphenicol or nisin to be selective for recipient cells when they are L. cremoris MG1614, L. cremoris NZ9000::pJP005 or L. cremoris LL64983, respectively. This selection should not select the donor strain. The presence of transconjugated plasmid in the recipient strain can be confirmed via culturing in the presence of the relevant phage against which the plasmid confers resistance. Alternately the presence of the plasmid can be verified using specific primers. Several of such primers are listed in Table 2 and in the sequence listing. The person skilled in the art is well aware how to design amplification primers for the detection of a polynucleotide of interest. The method can also be performed by means of the spread solid mating approach as described by Ortiz Charneco et al. (2021), using sequences according to the invention instead of sequences as described therein.

The invention also provides the isolated Lactococcus strain obtainable by the method described above.

### Further Applications and Products

The invention further provides for the use of the isolated strain as described above, or of the starter culture as described above, for the production of a fermented food product. This food product is preferably a fermented milk product.

Provided is further a method for the production of a fermented food product, the method preferably comprising the step of fermenting a substrate with an isolated strain as described above, or with the starter culture as described above, wherein the method is preferably performed in a non-sterile environment. In some embodiments the method is performed in a sterile environment. A substrate is preferably a dairy substrate such as a milk substrate. Also provided is a fermented food product obtainable by this method, wherein the fermented food product is preferably a fermented milk product, preferably as described elsewhere herein, more preferably a cheese product, preferably a cheese product as described elsewhere herein.

The invention also provides a kit of parts comprising a forward primer and a reverse primer, wherein the primers are complementary to any one of nucleotide sequences SEQ ID NOs: 1-10 and/or 55-67. Some of the primer sets provided herein detect two genes in tandem such as SEQ ID NO:s 9, 10, 25 and 26, and 27 and 28. Some of the primer set provided herein three genes in tandem such as SEQ ID NO;s 57, 58 and 58, and 62, 63 and 64. Preferably both primers are complementary to the same SEQ ID NO. A skilled person knows how to design a primer, which is preferably an oligonucleotide having a length that is preferably in the range of about 10 to about 50 nucleotides, more preferably about 12-40 nucleotides, more preferably about 15-30 nucleotides, most preferably about 18-24 nucleotides. The primers are preferably complementary to sequences within a SEQ ID NO that are about 50 to 500 nucleotides apart, more preferably about 100 to 300. Examples of primers and primers and primer sets that can be used for detection of the sequences of the invention are depicted in Table 2 and are, for example, SEQ ID NO: 33 and 34, SEQ ID NO: 41 and 42, SEQ ID NO: 43 and 44, SEQ ID NO: 45 and 46, SEQ ID NO: 47 and 48, SEQ ID NO: 45 and 48, SEQ ID NO: 49 and 50, SEQ ID NO: 51 and 52, SEQ ID NO: 49 and 52, SEQ ID NO: 53 and 54, SEQ ID NO: 81 and 82, SEQ ID NO: 83 and 84, SEQ ID NO: 85 and 86, SEQ ID NO: 87 and 88, SEQ ID NO: 89 and 90, SEQ ID NO: 91 and 92). This kit of parts or the primers as described herein can advantageously be used for detecting an isolated strain according to the invention; such strain may be a strain constructed as described herein or may be a naturally occurring strain.

Accordingly, there is provided for a method for the detection and/or isolation of a constructed or naturally occurring strain comprising an Abi system, or a component thereof, according to the invention. As an example, a sample comprising or suspected of comprising a constructed or naturally occurring strain, preferably a strain according to the invention, can be plated on a suitable substrate such as an agar plate, after which colony PCR can be performed using primers as described herein. This can detect a sequence of an Abi system, or a component thereof, according to the invention, and thus confirm presence of a strain according to the invention. Subsequently, such identified strain can be isolated. Preferably, such strain is a lactococcal strain as defined elsewhere herein and such strain can preferably be used in the methods and uses according to the invention.

### EXAMPLES

Research on over 300 plasmid sequences comprising many thousands of coding genes, derived from many different lactococcal strains resulted in the selection of more than 100 candidate genes belonging to over 82 different gene families for follow-up testing. These selected sequences were functionally cloned into plasmids with constitutive and/or inducible promoters to allow expression of the candidate genes after introduction within a lactococcal tester strain.

Surprisingly, a number of these candidate genes were able to confer phage resistance upon introduction. Upon testing individual genes, sequences which originally reside on lactococcal plasmids resulted in the identification of several novel lactococcal phage resistance and/or Abi mechanisms (SEQ ID NOS: 1-10). These systems conferred high resistance against phages of the genera Skunavirus and/or Ceduovirus in both solid and liquid media, regardless of the growth phase of the culture. The phage resistance and Abi systems, for example can be encoded on conjugative plasmids, allowing these anti-phage activities to be transferred by conjugation from a donor to an acceptor strain to generate robust starter strains with enhanced phage-resistance properties.

### Example 1—Materials & Methods

### 1.2 Media and Growth Conditions

The bacterial strains, plasmids and bacteriophages used in this study are listed in Table 3. Lactococcal strains used in this study were grown overnight at 30° C. in M17 broth (Oxoid, Basingstoke, Hampshire, United Kingdom) supplemented with 0.5% (w/v) glucose (GM17) for 16-20 hours. For the preparation of GM17 agar plates 1.5% (w/v) bacteriological agar was added. GM17 was supplemented with either chloramphenicol (5 μg/ml, to select for strains carrying pNZ44 or its derivatives), tetracycline (5 μg/ml, to select for strains harboring pPTPi or its derivatives), erythromycin (10 μg/ml, for pPEPi- or derivative-containing strains) or streptomycin (500 μg/ml, to select for L. cremoris MG1614). Nisin from L. lactis (1-10 ng/ml, Sigma-Aldrich, Gillingham, UK) was added to growing cultures when an OD_{600 nm} of 0.2-0.3 was reached for the induction of the PnisA promoter of pPTPi-derivative plasmids. Where appropriate, GM17 agar plates were also supplemented with nisin as described above.

TABLE 3
Bacterial strains, plasmids and bacteriophages used in this study

| | | |
|---|---|---|
| Bacterial strain, | | |
| plasmid or | | |
| bacteriophage | Characteristics* | Source or Reference |

Lactococcal strains

| | | |
|---|---|---|
| L. cremoris | Plasmid-free, host for cloning & for | (Linares et al., 2010) |
| NZ9000 | bacteriophage assays | |
| L. cremoris 3107 | Plasmid-free, host for cloning & bacteriophage | (Erazo Garzon et al., |
| | assays | 2019) |
| L. lactis IL1403 | Plasmid-free, host for cloning & bacteriophage | (Bolotin et al., 2001) |
| | assays | |
| L. cremoris | Lactococcal strain carrying pPTPL-sk1cos for the | (Mahony et al., 2008) |
| MG1363 | preparation of the transduction lysate | |
| L. cremoris | Plasmid free strain, used as recipient for | (Gasson, 1983) |
| MG1614 | conjugation, Str^{r} | |
| LL64983 | L. lactis commercial strain, recipient for mating | DSM culture collection |
| | experiments | |
| LL69075 | L. cremoris commercial strain, donor for mating | DSM culture collection |
| | experiments | |

Plasmids (SEQ ID NO)

| | | |
|---|---|---|
| pNZ44 | High-copy number vector for cloning, Cm^{r} | (McGrath et al., 2001) |
| pPTPi | Nisin-inducible, low-copy number vector for | (O'Driscoll et al., 2004) |
| | cloning, Tc^{r} | |
| pPEPi | Nisin-inducible, low-copy number vector for | |
| | cloning, Em^{r} | |
| | Low-copy, tetracycline-resistant vector | |
| PPTPL-sk1cos | encompassing the cos site of phage sk1. Used | (Mahony et al., 2008) |
| | for the transduction assays | |
| pJP005 | Plasmid encoding RecT and chloramphenicol | Van Pijkeren & Britton, |
| | resistance | 2012 |

Bacteriophages

| | | |
|---|---|---|
| sk1 | Skunavirus propagated on L. cremoris NZ9000 | (Chandry et al., 1994) |
| 712 | Skunavirus propagated on L. cremoris NZ9000 | (Rincé et al., 2000) |
| jj50 | Skunavirus propagated on L. cremoris NZ9000 | (Josephsen, 1989) |
| p2 | Skunavirus propagated on L. cremoris NZ9000 | (Bebeacua et al., 2013) |
| c2 | Ceduovirus propagated on L. cremoris NZ9000 | (Higgins et al., 1988) |
| 66901 | Skunavirus propagated on L. cremoris 3107 | (Oliveira et al., 2018) |
| 62601 | Skunavirus propagated on L. cremoris 3107 | (Oliveira et al., 2018) |
| 69075 | Skunavirus propagated on L. cremoris 3107 | DSM culture collection |
| TP901-1 | P335 phage propagated on L. cremoris 3107 | (Christiansen et al., |
| | | 1994) |
| LC3 | P335 phage propagated on L. cremoris 3107 | (Lillehaug et al., 1991) |
| Dub35A | P335 phage propagated on L. cremoris 3107 | (Mahony et al., 2017) |
| 63301 | P335 phage propagated on L. cremoris 3107 | (Oliveira et al., 2016) |
| P087 | P087 phage propagated on L. cremoris 3107 | (Villion et al., 2009) |
| P008 | Skunavirus propagated on L. lactis IL1403 | (Loof et al., 1983) |
| bIL170 | Skunavirus propagated on L. lactis IL1403 | (Crutz-Le Coq et al., |
| | | 2002) |
| bIL66 | Skunavirus propagated on L. lactis IL1403 | (Bidnenko et al., 1995) |
| P113G | Skunavirus propbagated on L. lactis IL1403 | (Dupont et al., 2005) |
| 340 | Skunavirus propagated on L. lactis IL1403 | (Mahony et al., 2013) |

*Str^{r}, streptomycin resistant; Cm^{r}, chloramphenicol resistant; Tc^{r}, tetracycline resistant; Em^{r}, erythromycin resistant

### 1.2 Molecular Cloning of Candidate Phage Defence-Encoding Genes

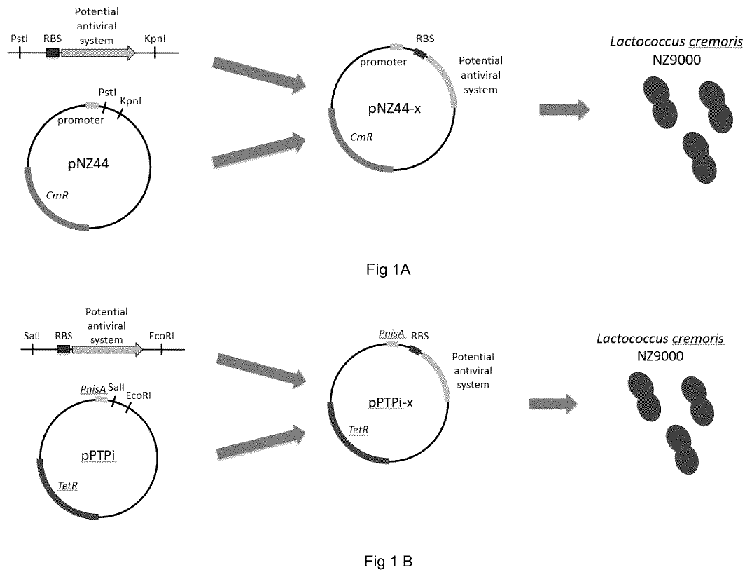

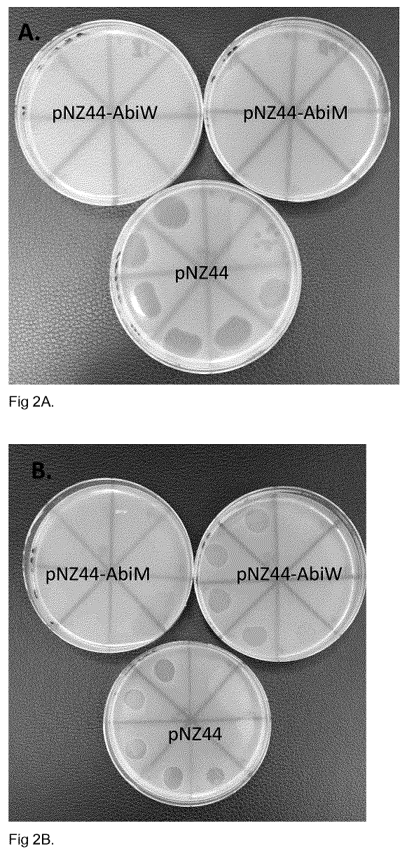

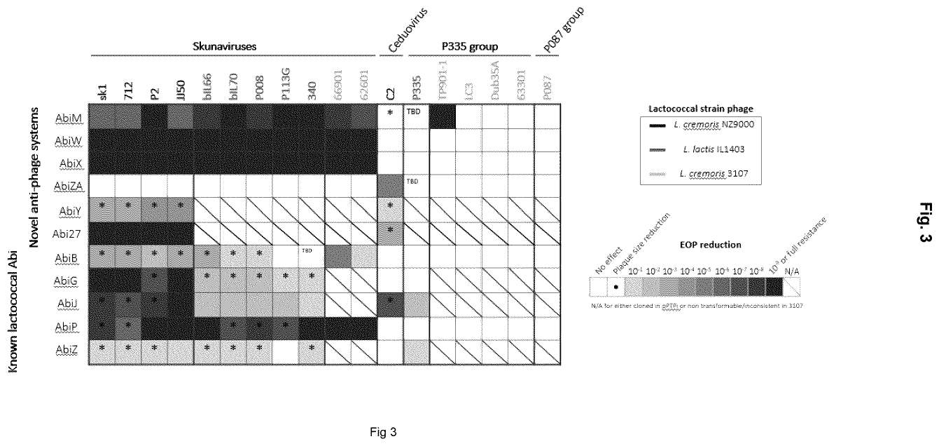

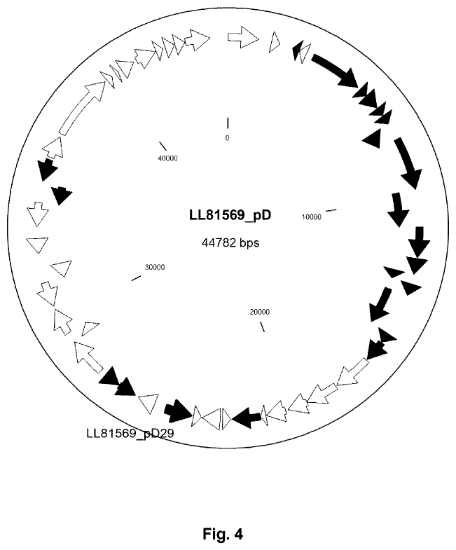

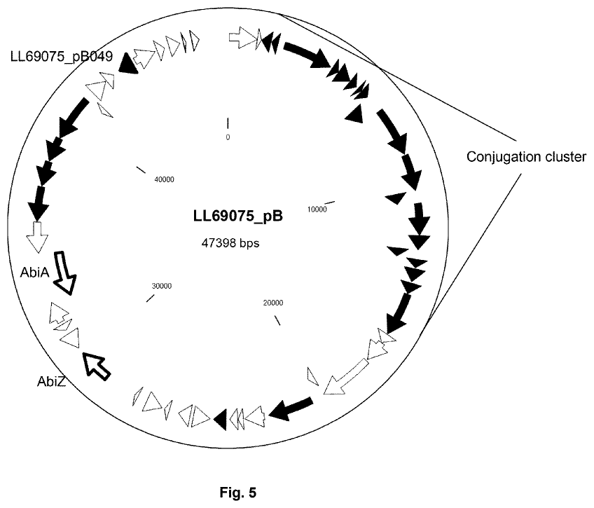

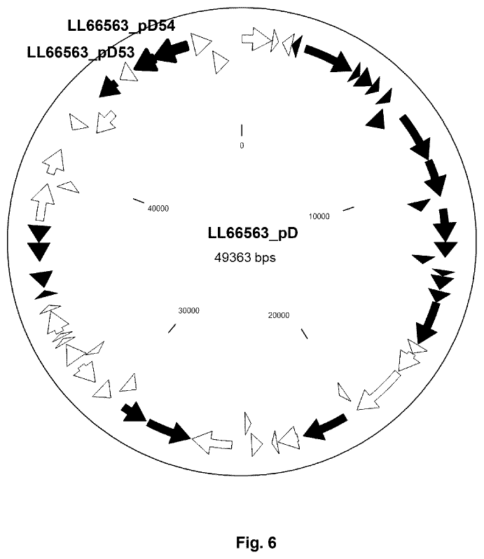

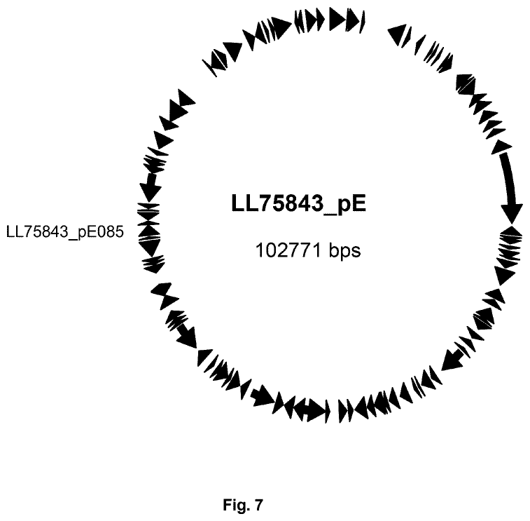

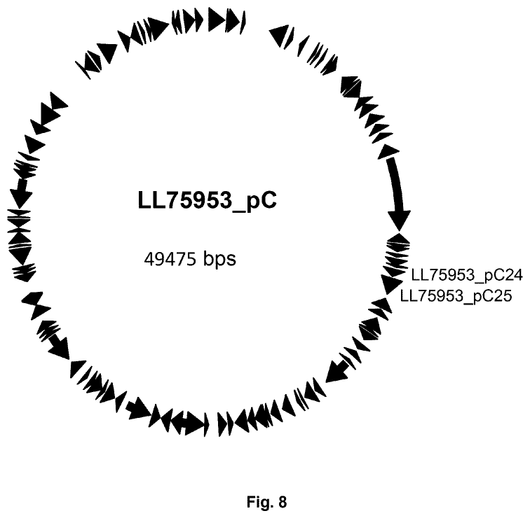

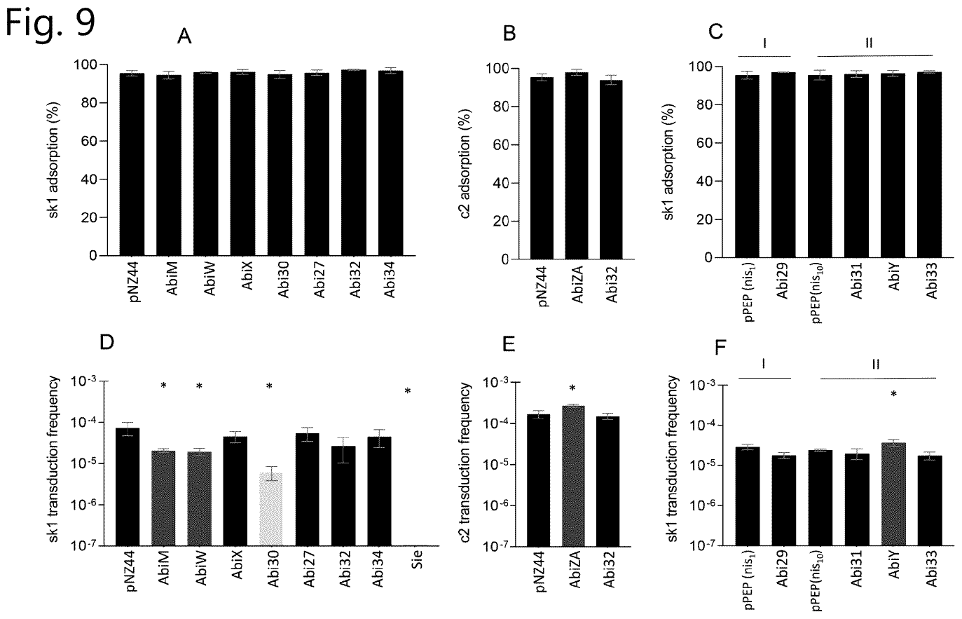

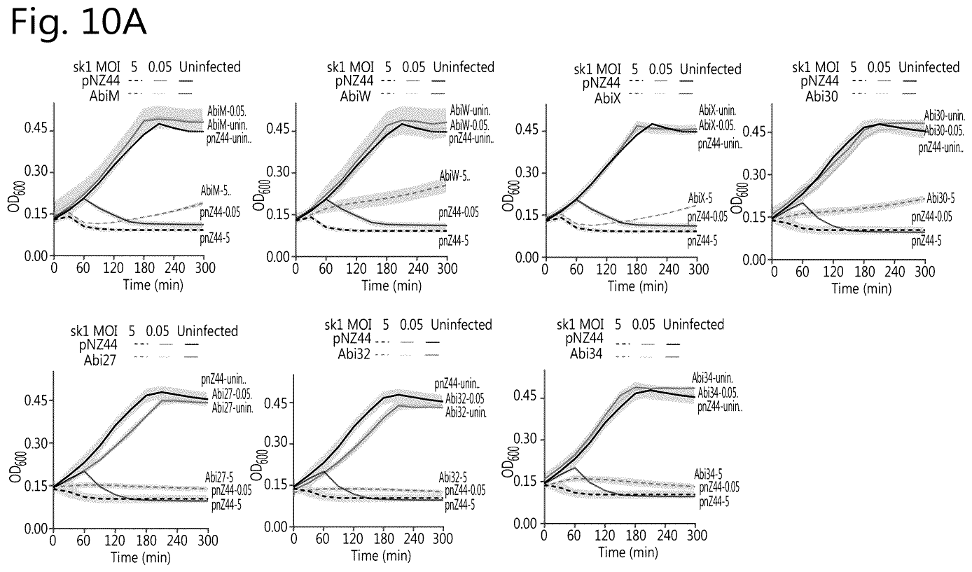

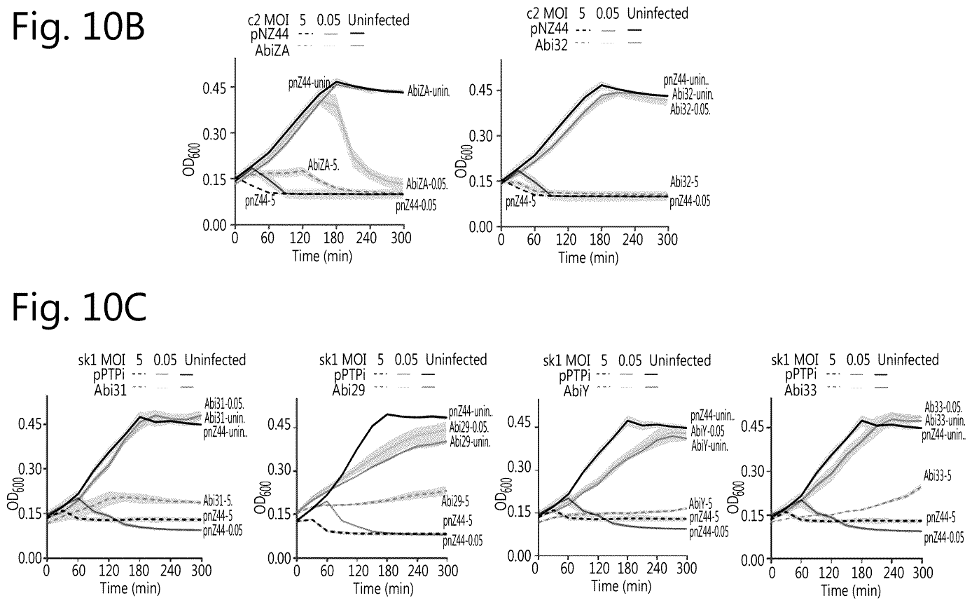

DNA fragments to be cloned were amplified using Phusion Green High-Fidelity DNA Polymerase (Thermo Fisher Scientific, Waltham, MA, United States) according to the manufacturer's instructions and with relevant primers. The initial denaturation step was performed for 10 min to allow cell disruption and release of the template DNA. The high-copy, constitutive-expression lactococcal plasmid pNZ44 was used as a cloning vector, although in some cases DNA fragments, which were recalcitrant to cloning in pNZ44, were instead cloned in the low-copy, nisin-inducible vector, pPTPi (Table 3) in a manner as also depicted in FIG. 1. FastDigest restriction enzymes, including PstI, KpnI, XbaI, HindIII, SalI, SacI, EcoRI, and T4 DNA ligase (Promega, Hampshire, United Kingdom) were used according to the instructions of the manufacturer. Restricted DNA fragments and corresponding cloning vector backbones were purified prior to ligation using the GenElute PCR Clean-up Kit (Sigma-Aldrich). Ligation mixtures were introduced into L. lactis NZ9000 by electroporation (see next paragraph) and transformants were selected based on either chloramphenicol or tetracycline (for the presence of [derivatives of] pNZ44 or pPTPi, respectively). Transformants carrying the desired recombinant plasmids were screened by colony PCR using pNZ44_F 5′-CTAATGTCACTAACCTGCCCCG-3′ (SEQ ID NO: 29) and pNZ44_R 5′-GCTTTATCAACTGCTGCT-3′ (SEQ ID NO: 30) or pPTPi_F 5′-TGATTTCGTTCGAAGGAACTA-3′ (SEQ ID NO: 31 and pPTPi_R 5′-TGGCGGACAATAAGTCCTC-3′ (SEQ ID NO: 32 primers. Plasmid DNA was extracted from the positive clones using the GeneJET Plasmid Miniprep/Maxiprep Kit (Thermo Scientific, Waltham, MA, USA) according to the manufacturer's instructions with some modifications as follows. Harvested cells were resuspended in TE buffer (10 mM Tris, 1 mM EDTA, pH 7.5) containing 25% sucrose and 30 mg/mL lysozyme (Sigma Aldrich) and incubated for 30 min at 37° C. prior to the plasmid DNA extraction procedure. The sequence integrity of the recombinant plasmids was verified by Sanger sequencing (Eurofins, Ebersberg, Germany).

Electrocompetent cells were prepared by inoculating 40 ml of GM17 supplemented with 0.5 M sucrose and 1% (for L. cremoris 3107) or 1.5% glycine (for L. cremoris NZ9000 and L. lactis IL1403) with up to 5% of fresh or pre-adapted overnight culture. The culture was incubated at 30° C. until an OD_{600 nm} of 0.5 was reached. Cells were harvested, washed, aliquoted and stored as previously described (Holo and Nes, 1989). 45 μl of competent cell suspension was mixed with 5 μl of the ligation mixture and electroporated at 2.0 kV, 200Ω and 25 μF in 0.2 cm cuvettes using an ECM 630 Exponential Decay Wave Electroporation System (BTX, Holliston, USA). After electroporation, cells were recovered in 950 μl GM17 broth containing 20 mM MgCl₂ and 2 mM CaCl₂ and incubated at 30° C. for 2.5 h. Bacteria were then plated on M17 supplemented with the relevant antibiotics for the selection of pNZ44 or pPTPi and incubated at 30° C. for 24-48 h.

### 1.3 Bacteriophage Propagation, Phage & Lysis-In-Broth Assays

Bacteriophages used in this study (Table 3) were propagated by adding 2-5% (v/v) of a fresh overnight culture of the appropriate lactococcal host strain, CaCl₂ (10 mM) and 1% (v/v) of the phage lysate or a single plaque in GM17 broth. Incubation was continued at room temperature or 30° C. until lysis occurred. The lysates were filtered (pore size 0.45 μm, Sarstedt AG & Co. KG, Nümbrecht, Germany) and stored at 4° C.

Spot and plaque assays were performed using the double agar method of Lillehaug (1997) with some modifications. Solid (bottom layer) and semi-solid agar (top layer) was prepared using GM17 medium supplemented with CaCl₂ (10 mM); 1% bacteriological agar was incorporated in the solid layer, and 0.4% agar was used included in the semi-solid agar. SM buffer (10 mM CaCl₂, 100 mM NaCl, 10 mM MgSO₄, 50 mM Tris-HCl at pH 7.5) was used as the diluent in all the bacteriophage assays. The efficiency of plaquing (EOP) was determined by dividing the titre (pfu/ml) of the test strain by that of the control strain.

Lysis-in-broth assays were performed by infecting 10 ml cultures of the lactococcal host strain NZ9000 carrying either the empty vector or the vector with a novel phage-resistance system, to an optical density at 600 nm (OD_{600 nm}) of 0.2 with sk1 phage lysate at a multiplicity of infection (MOI) of 0.1 or 5. Cells and phages were incubated at 30° C. and OD_{600 nm} was recorded at 15 min intervals for a total of 90 min. Uninfected cultures were included as control.

### 1.4 Phage Escape Mutant Isolation & Analysis

Strains were challenged with high titre lysates (≥10⁹ pfu/ml) of skunaviruses or ceduoviruses. Single plaques of spontaneous phage mutants were isolated from plates containing the novel defence systems and propagated on the relevant strain carrying the phage-resistance system or, if not possible, on the wild-type strain. To improve phage plaque visualization, 0.5% glycine was added to top agar or agarose 0.2% was used to replace agar. The phage genomic DNA was extracted using the Phage DNA extraction kit (Norgen, Thorold, Canada) as recommended by the manufacturer. The genomes of the phage escape mutants were sequenced using Illumina MiSeq technology (GenProbio, Parma, Italy). MIRA (Mimicking Intelligent Read Assembly) version 4.0.2 was used for de novo assembly of MiSeq-derived phage genome sequences to generate a consensus sequence. Open reading frames (ORFs) were predicted using a combination of Prodigal version 2.6 and BLASTX (Gish & States, 1993; Hyatt et al., 2010), followed by manual assessment, curation, and correction of predicted ORFs. Functional annotations were generated using BLASTP (Altschul et al., 1997) analysis against the nonredundant protein database (nr) provided by the National Center for Biotechnology Information (NCBI; available on the internet at blast.ncbi.nlm.nih.gov/Blast.cgi), as well as using the MEGAnnotator pipeline (Lugli et al., 2016). Proposed protein functions were validated by querying protein domain database Pfam (Bateman et al., 2004) and the NCBI Conserved Domain Database (Marchler-Bauer et al., 2011), and by performing homology prediction searches using HHpred (Söding et al., 2005). The generated sequences were analysed for the presence of single nucleotide polymorphisms (SNPs) when compared to the wild-type genome sequence.

### 1.5 Adsorption & Transduction Assays

Adsorption assays were carried out as described by Garvey et al. (1996) with some modifications. Specifically, host cells were grown at 30° C. until OD_{600 nm}~0.7, while CaCl₂ was added to a final concentration of 10 mM.

Transduction assays were based on the protocol of McGrath et al. (2001). Briefly, the transduction lysate was prepared by infecting the lactococcal strain MG1363 harbouring PPTPL-sk1 cos (Table 3) with phage sk1 lysate. Transduction of host cells was determined by adding the transduction lysate at a multiplicity of infection (MOI) of 0.1 and CaCl₂ to a final concentration of 10 mM, while the plates were supplemented with 5 μg/ml tetracycline for the selection of the transductants. All experiments were performed in triplicate and data are presented as means±standard deviation (SD).

### 1.6 Conjugation Experiments

Conjugation was performed by means of the spread solid mating approach as described by Ortiz Charneco et al. (2021). A single selection strategy was adopted as there were no other selectable characteristics present on the plasmids carrying the novel anti-phage traits (e.g. resistance to antibiotics), that could be used when the latter are transferred to the recipient strains. Therefore, the agar plates were supplemented with either streptomycin, chloramphenicol or nisin to be selective for the recipient cells (for L. cremoris MG1614, L. cremoris NZ9000::pJP005 and LL64983, respectively), but not for the plasmid (pLL69075B).

The presence of pLL69075B in L. cremoris MG1614, L. cremoris NZ9000::pJP005 and LL64983 was further verified by using pLL69075B- and NZ9000/MG1614/LL64983-specific primers (SEQ ID NOS: 48-55), confirming conjugative transfer of pLL69075B to the recipient strain. In a similar way the presence of respective plasmids and/or genes and sequence of the invention can be detected/confirmed by PCR using the primer sets as referred to above and below and/or as listed in SEQ ID NO's: 33-54.

### Example 2—Results

### 2.1 Selection of Putative Plasmid-Encoded Phage Defence Systems

To identify novel, non-RM (i.e. restriction-modification)-based phage defence systems encoded by lactococcal plasmids, a total of 321 plasmid sequences derived from 53 sequenced lactococcal strains were analysed. Genes that encode proteins with significant similarity to previously characterized Abi systems (e.g. AbiA, AbiB, AbiP) were also included as positive controls. The above applied criteria resulted in the identification of >100 candidate phage defence-encoding genes representing many different gene families. In most cases, the candidate phage-defence systems were represented by a single gene, though in some cases where adjacent genes fulfilled the selection criteria such adjacent genes were cloned and tested together.

### 2.2 Identification of Novel Phage Defence Systems

The more than one-hundred selected candidate genes or gene pairs, were cloned in pNZ44 or pPTPi and introduced into L. cremoris NZ9000 (Approached as summarized in FIG. 1). Following verification of the resulting recombinant plasmids, each of the resulting strains was challenged with phages to determine if the presence of a given recombinant plasmid was associated with phage-resistance.

Spot assays provided a preliminary overview of the phage sensitivity profile of the strains carrying the selected candidate phage defence encoding-genes. Among the tested genes/gene pairs, twelve were shown to be associated with resistance against the tested phages. Six of these were previously established Abi systems (AbiA, AbiB, AbiG, AbiJ, AbiP and AbiZ) that were identified during the candidate gene selection and included as positive controls, confirming the validity of the experimental approach.

The remaining sequences that were associated with a phage-resistance phenotype were identified as novel phage defence systems that were subsequently named AbiM, AbiW, AbiX, AbiY, AbiZA & Abi27 (as they fulfil the criteria for Abi systems, see below), representing the products of the genes LL79476_pB052, LL81569_pD29, LL69075_pB49, LL66563_pD53 &_pD54, LL75843_pE085 and LL75953_pC24 & pC25, respectively (Table 4).

TABLE 4
Characteristics of the novel Abi systems

| | | Length | GC | MW | Isoelectric | TM | Prevalence |
|---|---|---|---|---|---|---|---|
| Abi | Strain/Plasmid/ORF | (aa) | (%) | (kDa) | point | helices | (strains) |
| AbiM | LL79476_pB52 | 303 | 28 | 35 | 9.6 | 0 | L. lactis, Lactobacilloae, |
| | | | | | | | Lactobacillies, L. cremoris |
| AbiW | LL81569_pD29 | 316 | 28 | 37 | 5.3 | 0 | L. lactis & L. cremoris |
| AbiX | LL69075_pB49 | 180 | 31 | 22 | 4.1 | 0 | L. lactis & L. cremoris |
| AbiYi | LL66563_pD54 | 453 | 23 | 53 | 7.2 | 0 | L. lactis & L. piscium |
| AbiYii | LL66563_pD53 | 280 | 26 | 33 | 4.5 | 0 | L. lactis & L. piscium |
| AbiZA | LL75843_pE085 | 392 | 27 | 47 | 9.7 | 4 | L. lactis & L. piscium |
| Abi27a | LL75953_pC24 | 190 | 28 | 22 | 5.7 | 2 | L. lactis & L. piscium |
| Abi27b | LL75953_pC25 | 408 | 30 | 47 | 8.9 | 0 | L. lactis & L. piscium |

AbiM, AbiW AbiX and abiZA are encoded as single genes that were cloned in the high-copy number vector pNZ44, whereas AbiY and Abi27 were one of the genes that also were cloned in the nisin inducible vector pPTPi instead. AbiY and Abi27 was considered a potential two gene phage defence system, as the stop codon of the first gene [abiYᵢ, abi27ₐ] is very close to the start of second gene [abiYᵢᵢ, abi27_{b}]. For all systems a spot test (one example for AbiM and AbiW systems is shown in FIG. 2) was performed to assess potential phage resistance. Surprisingly phage resistance was detected with various profiles. To test for the 2 system genes, if both genes are required to confer the phage defence phenotype, the genes were also cloned separately in pPTPi. Gene expression was induced through the addition of nisin, and phage-resistance was observed only when both genes were present (data not shown), confirming that AbiY and Abi27 represents a novel, two-protein lactococcal phage defence system (Table 4).

Table 4 summarizes some of the salient properties of the novel putative Abi systems. In FIG. 4 through 8, the respective plasmid maps of the plasmids derived of the sequenced lactococcal strains and the relative positions of the Abi genes on the plasmids are indicated. The protein length, the molecular weight and the isoelectric point, varies greatly among them. Interestingly, these genes are rare among the gene families tested.

Based on the comparative sequence analysis (BLASTN & BLASTP) of the novel putative Abi systems against available sequence data on NCBI database and on Pfam for the identification of any identifiable functional domains, AbiM, AbiW, AbiX, abiZA, AbiY and Abi27 are hypothetical proteins with no significant homology to any previously described phage defence system. AbiW and AbiYᵢᵢ proteins contain domains of unknown function (DUF2971 and DUF4435, respectively). AbiX contains a YfbU protein domain, that has been reported to be involved in cell death when triggered by DNA damage (Amitai et al., 2009). No significant protein domain predictions were found for AbiM. AbiYᵢ (LL66563_pD54) is predicted to contain an AAA (ATPases Associated with a variety of cellular Activities) domain, which are found in members of the AAA+ protein superfamily. These proteins are involved in various cellular processes and have in some instances been shown to be responsible for conformational changes in targeted proteins (Frickey & Lupas, 2004; Hanson & Whiteheart, 2005). Comparative protein domain analysis for Abi27a&27b suggest a Type I CBASS (cyclic oligonucleotide-based antiphage signalling system) consisting of 2 factors wherein cyclic nucleotide signals induce cell death. The Abi27a factor could be the transmembrane effector and Abi27b would produce the signal.

### 2.3 Anti-Phage Spectrum of the Newly Identified Phage Defence Systems

To evaluate if the antiphage activity of the novel phage-resistance systems is associated with a specific phage (e.g. sk1, bIL66 or c2) or with a whole phage group (i.e. Skunavirus, Ceduovirus, P335 & P087 phage groups), various lactococcal bacteriophages representing the afore-mentioned groups were employed in this study (Table 3). Furthermore, since L. cremoris NZ9000 is sensitive to just a small number of phages of the genera Skunavirus and Ceduovirus, the pNZ44-derivatives carrying the novel phage defence genes were introduced into L. cremoris 3107 and L. lactis IL1403, allowing assessment of the anti-phage activity spectrum using various Skunavirus and Ceduovirus phages and phages belonging to the P335 and P087 groups.

Table 5 summarizes the efficiency of plaquing (EOP) of each employed phage on the lactococcal strain (i.e., L. cremoris NZ9000, L. cremoris 3107, L. lactis IL1403) harbouring each of the novel Abi systems compared to the wild type strain carrying the empty vector (pNZ44 or pPTPi). Interestingly, the novel systems exhibited high resistance against phages of the Skunavirus genus and in some cases against the Ceduovirus genus or even both. The Skunavirus genus is the most frequently encountered phage genus in the dairy industry (Mahony et al., 2012). AbiZA was shown to provide resistance against the C2 Ceduovirus. AbiW, AbiX, Abi27 and AbiY were shown to provide the highest level of anti-phage activity against the Skunavirus group. Resistance was recorded against all tested phages of the Skunavirus genus, highlighting the broad effectiveness of these novel systems against this problematic group of phages. AbiM, Abi27 and AbiY exhibited partial resistance against Ceduovirus c2, with a plaque size reduction (approx. 50% reduction) or both EOP and plaque size reduction, respectively. AbiM was unique in providing resistance specifically against a single P335 phage, namely TP901-1, in addition to the resistance against all tested phages of the Skunavirus genus. Similar levels of anti-phage activity were recorded when the strains carrying the novel Abi systems were challenged with phage sk1 at early log (OD_{600 nm}~0.5) instead of stationary phase (Table 5). Additionally, in congruence with the plaque assay results, the novel phage defence systems confer phage resistance in liquid media against the Skunavirus sk1 (see below, 3.5 paragraph). A summary of all resistances as tested by efficiency of plaquing (EOP) results including the comparison of resistance profile with 5 known Abi (AbiB, AbiG, AbiJ, AbiP and AbiZ) systems is shown in FIG. 3. From this overview and profiles, it is clear that the identified novel anti-phage Abi systems have very different resistance profiles compared to known Abi systems. The darker colour indicates an increased resistance and illustrates that the new Abi systems have an improved resistance compared to the known Abi systems.

TABLE 5
Efficiency of plaquing (EOP) of different phage group representatives (Skunavirus,
Ceduovirus, P335 & P087 phage group) on different lactococcal hosts (NZ9000, 3107, IL
1403) carrying the novel phage-resistance systems (AbiM, AbiW, AbiX, AbiZA, AbiY & Abi27)

| Phage | Lactococcal strain | | | | |
|---|---|---|---|---|---|
| | NZ9000::pNZ | NZ9000::pNZ44 + | NZ9000::pNZ | NZ9000:pNZ | NZ9000::p |
| | 44/ | AbiM | 44 + | 44 + | PTPi + |
| | NZ9000::pPT | | AbiW | AbiX | AbiY |
| | Pi | | | | |
| | Phage group: | Skunavirus | | | |
| sk1 | 1 | 1.48 × 10⁻⁶ ± | ≤1.25 × 10⁻⁷ | ≤3.4 × 10⁻⁸ | ≤3.4 × 10⁻⁸ |
| | | 0.58 × 10⁻⁶ | | | |
| 712 | 1 | 1.14 × 10⁻⁶ ± | ≤1.66 × 10⁻⁷ | ≤4.9 × 10⁻⁸ | ≤4.9 × 10⁻⁸ |
| | | 0.04 × 10⁻⁶ | | | |
| p2 | 1 | ≤1.17 × 10⁻⁷ | ≤2.5 × 10⁻⁷ | ≤7.24 × 10⁻⁸ | ≤7.24 × 10⁻⁸ |
| JJ50 | 1 | 1.48 × 10⁻⁶ ± | ≤7.1 × 10⁻⁸ | ≤8.31 × 10⁻⁸ | ≤8.31 × 10⁻⁸ |
| | | 0.35 × 10⁻⁶ | | | |
| | 3107::pNZ44 | 3107::pNZ44 + | 3107::pNZ44 + | 3107::pNZ44 + | |
| | | AbiM | AbiW | AbiX | |
| 66901 | 1 | 3.11 × 10⁻⁸ ± | ≤3.11 × 10⁻⁹ | ≤7.11 × 10⁻⁹ | |
| | | 3.11 × 10⁻⁸ | | | |
| 62601 | 1 | 2.81 × 10⁻⁷ ± | ≤1.20 × 10⁻⁷ | ≤2.80 × 10⁻⁸ | |
| | | 0.46 × 10⁻⁷ | | | |
| | IL1403::pNZ4 | IL1403::pNZ44 + | IL1403::pNZ | IL1403::pNZ | |
| | 4 | AbiM | 44 + | 44 + | |
| | | | AbiW | AbiX | |
| bIL66 | 1 | 8 × 10⁻⁹ ± | ≤6.12 × 10⁻⁹ | ≤6.12 × 10⁻⁹ | |
| | | 2.81 × 10⁻⁹ | | | |
| bIL70 | 1 | ≤3.51 × 10⁻⁸ | ≤3.51 × 10⁻⁸ | ≤3.51 × 10⁻⁸ | |
| P008 | 1 | 6.68 × 10⁻⁷ ± | ≤1.16 × 10⁻⁸ | ≤1.16 × 10⁻⁸ | |
| | | 0.08 × 10⁻⁹ | | | |
| P113G | 1 | ≤1.39 × 10⁻⁸ | ≤1.39 × 10⁻⁸ | ≤1.39 × 10⁻⁸ | |
| 340 | 1 | ≤2.42 × 10⁻⁸ | ≤2.42 × 10⁻⁸ | ≤2.42 × 10⁻⁸ | |
| | 3107::pNZ44 | 3107::pNZ44 + | 3107::pNZ44 + | 3107::pNZ44 + | |
| | | AbiM | AbiW | AbiX | |
| | Phage group: | P335 | | | |
| TP901-1 | 1 | ≤3.73 × 10⁻⁸ | ≈1 | ≈1 | |
| LC3 | 1 | ≈1 | ≈1 | ≈1 | |
| Dub35A | 1 | ≈1 | ≈1 | ≈1 | |
| 63301 | 1 | ≈1 | ≈1 | ≈1 | |
| | NZ9000::pNZ | NZ9000::pNZ44 + | NZ9000::pNZ | NZ9000::pNZ | NZ9000::p |
| | 44/ | AbiM | 44 + | 44 + | PTPi + |
| | NZ9000::pPT | | AbiW | AbiX | AbiY |
| | Pi | | | | |
| | Phage group: | Ceduovirus | | | |
| c2 | 1 | Size-reduction | ≈1 | ≈1 | 6.54 × 10⁻² ± |
| | | | | | 2.56 × 10⁻² |
| | 3107::pNZ44 | 3107::pNZ44 + | 3107::pNZ44 + | 3107::pNZ44 + | |
| | | AbiM | AbiW | AbiX | |
| | Phage group: | P087 | | | |
| P087 | 1 | ≈1 | ≈1 | ≈1 | |

Efficiency of plaquing (EOP) of phage sk1 (Skunavirus) on lactococcal host L. cremoris NZ9000 carrying novel phage-resistance systems (AbiM, AbiW, AbiX, & AbiY) at an early log (OD₆₀₀~0.5)

| | Lactococcal strain | | | | |
|---|---|---|---|---|---|
| | NZ9000::pNZ | | | | |
| | 44/ | | NZ9000::pNZ4 | NZ9000::pNZ4 | NZ9000::pPT |
| | NZ9000::pPT | NZ9000::pNZ44 + | 4 + | 4 + | Pi + |
| Phage | Pi | AbiM | AbiW | AbiX | AbiY |
| sk1 | 1 | 2.95 × 10⁻⁷ ± 0.52 × 10⁻⁷ | ≤1.80 × 10⁻⁸ | ≤1.80 × 10⁻⁸ | 2.44 × 10⁻⁴ ± |
| | | | | | 0.83 × 10⁻⁴ |

Efficiency of plaquing (EOP) of phage sk1 (Skunavirus) and C2 viruses on lactococcal host L. cremoris NZ9000 carrying novel phage-resistance system (Abi27) at an early log (OD₆₀₀~0.5)

| Strain | Phage family | Phage | EOP |
|---|---|---|---|
| NZ9000 | Skunavirus | sk1 | <2.81 ± 0.19 × 10⁻⁹ |
| | | p2 | <3.61 ± 0.49 × 10⁻⁸ |
| | | JJ50 | <1.01 ± 0.02 × 10⁻⁷ |
| | | 712 | <2.82 ± 0.56 × 10⁻⁸ |
| | Ceduovirus | c2* | 1.12 ± 0.46 × 10⁻³ |
| | | 50501 | <2.69 ± 0.74 × 10⁻⁷ |
| | | 60301 | <2.65 ± 0.52 × 10⁻⁸ |
| | | 62606 | <2.96 ± 0.47 × 10⁻⁸ |

*plaque size reduction

Efficiency of plaquing (EOP) of phage sk1 (Skunavirus) and C2 viruses on lactococcal host L. cremoris NZ9000, 3107 and IL1403 carrying novel phage-resistance system (AbiZA) at an early log (OD₆₀₀~0.5)

| Strain | Phage family | Phage | EOP |
|---|---|---|---|
| NZ9000 | Skunavirus | | N.A. |
| | Ceduovirus | c2* | 7.78 ± 1.89 × 10⁻⁵ |
| | | 50501* | 6.53 ± 4.36 × 10⁻⁷ |
| | | 60301* | 4.50 ± 2.31 × 10⁻⁷ |
| | | 62606 | 2.30 ± 0.64 × 10⁻⁵ |
| 3107 | Skunavirus | | N.A. |
| | Ceduovirus | c2* | 1.67 ± 1.11 × 10⁻⁵ |
| | | 50102 | 2.62 ± 0.80 × 10⁻⁵ |
| | | 50501* | 3.98 ± 3.62 × 10⁻⁶ |
| | | 60301* | 6.64 ± 0.95 × 10⁻⁶ |
| | | 62606 | 4.03 ± 2.30 × 10⁻⁶ |
| IL1403 | Skunavirus | | N.A. |
| | Ceduovirus | 50501 | <6.75 ± 2.05 × 10⁻⁶ |
| | | 56006* | 3.14 ± 1.29 × 10⁻⁶ |
| | | 60302 | <1.09 ± 0.34 × 10⁻⁵ |

*plaque size reduction

### 2.4 the Novel Phage-Resistance Systems are Active Post Phage DNA Injection

To examine if the novel phage-resistance systems impact adsorption or DNA injection stages of the phage cycle, adsorption and transduction assays were performed. L. cremoris NZ9000 harbouring pNZ44/pPTPi with or without the identified phage defence genes with sk1 as the test (/transducing) phage were used. Adsorption assays (Table 6) revealed that the systems do not interfere with the process of adsorption of phage sk1 (>91% adsorption). Similarly, DNA injection does not appear to be affected by the presence of the phage defence system, as the observed transduction frequency was similar to that obtained for the control strains (Table 6). Therefore, the novel phage resistance systems do not block, prevent or inhibit phage adsorption or DNA injection, indicating that they are intracellularly active and that they can be categorized as abortive infection systems.

TABLE 6
Adsorption and transduction of phage sk1 in L. cremoris
NZ9000 strains carrying the novel Abi systems (AbiM,
AbiW, AbiX, AbiZA, AbiY & Abi27) compared to the control
strains (L. cremoris pNZ44/pPTPi)

| L. cremoris | | Transduction |
|---|---|---|
| NZ9000 | Adsorption (%) | frequency |
| pNZ44 | 92.00 ± 1.36 | 6.09 × 10⁻⁴ ± 0.66 × 10⁻⁴ |
| pNZ44_AbiM | 96.02 ± 1.19 | 3.06 × 10⁻⁴ ± 0.62 × 10⁻⁴ |
| pNZ44_AbiW | 93.70 ± 1.40 | 4.12 × 10⁻⁴ ± 0.32 × 10⁻⁴ |
| pNZ44_AbiX | 94.63 ± 0.50 | 4.87 × 10⁻⁴ ± 0.75 × 10⁻⁴ |
| pNZ44_AbiZA | 97.84 ± 1.43 | N.A. |
| pPTPi/pPEPi | 93.86 ± 2.10 | 4.48 × 10⁻⁴ ± 0.47 × 10⁻⁴ |
| PPTPi/pPEPi_AbiY | 91.32 ± 2.95 | 8.46 × 10⁻⁴ ± 1.74 × 10⁻⁴ |
| pPTPi/pPEPi_Abi27 | N.A. | 2.01 × 10⁻⁴ ± 0.22 × 10⁻⁴ |

N.A.: Not analysed

### Lysis-In-Broth Experiments

To assess if the novel phage defence systems are abortive infection systems, lysis-in-broth assays in a low and a high multiplicity of infection (MOI) were performed. The results of such are summarized in Table 7 below. A comparison of the lysis profiles of L. cremoris NZ9000 harbouring pNZ44 in both uninfected cells and cells infected with sk1 and its derivatives bearing ID NO: 1 (AbiM) (a), SEQ ID NO: 2 (AbiW) (b), SEQ ID NO: 3 (AbiX) (c), or SEQ ID NOs: 4 (AbiZA) or SEQ ID NOs: 5 and 6 (AbiY) (d) or SEQ ID NOs: 7 and 8 (Abi27). OD₆₀₀ readings were taken at 15-min intervals. Each of the novel phage defence systems provides resistance against sk1 infection at an MOI=0.1, while the cultures demise at a high MOI (5), indicating that the novel defence systems are Abi. NZ9000 harbouring pNZ44 was used as a positive control. Each assay was performed in triplicate.

For the purpose of these experiments, the lysis profile of L. cremoris NZ9000 was recorded, carrying either the novel defence systems or the empty vector, with or without infection of sk1 at an OD_{600 nm} of 0.2 (MOI=0.1 or 5). In a low MOI infection, growth of the infected cultures carrying the novel phage defence system was unaffected by the phage infection, generating similar profiles to those of the uninfected strains, while cultures carrying the empty cloning vector pNZ44/pPTPi collapsed. However, at high MOI, bacterial cultures containing the novel defence systems demised, similar to cultures carrying the empty cloning vectors. According to the Abi phenotype, an infection of all cells carrying an Abi system would lead to its activation and therefore to bacterial death of nearly all cells. Consequently, the novel phage defence systems were characterised as abortive infection mechanisms.

TABLE 7
Lysis in broth - Characterization
of all Abi phage-resistance systems

| | Adsorption assay | | Transduction assay | | Lysis in broth | |
|---|---|---|---|---|---|---|
| Strain | Sk1 | C2 | Sk1 | C2 | Sk1 | C2 |
| pNZ44 | ++ | ++ | ++ | ++ | ++ | ++ |
| AbiM | ++ | N.A | ++ | N.A | ++ (high | N.A |
| | | | | | MOI) | |
| | | | | | −− (Low | |
| | | | | | MOI) | |
| AbiW | ++ | N.A | ++ | N.A | ++ (high | N.A |
| | | | | | MOI) | |
| | | | | | −− (Low | |
| | | | | | MOI) | |
| AbiX | ++ | N.A | ++ | N.A | ++ (high | N.A |
| | | | | | MOI) | |
| | | | | | −− (Low | |
| | | | | | MOI) | |
| AbiYi | ++ | N.A | ++ | N.A | ++ (high | N.A |
| | | | | | MOI) | |
| | | | | | −− (Low | |
| | | | | | MOI) | |
| AbiZA | N.A. | ++ | N.A. | ++ | N.A. | ++ (high |
| | | | | | | MOI) |
| | | | | | | +/− (Low |
| | | | | | | MOI) |
| Abi27 | ++ | ++ | ++ | ++ | ++ (high | ++ |
| | | | | | MOI) | |
| | | | | | −− (Low | |
| | | | | | MOI) | |
| pPTPi | ++ | ++ | ++ | ++ | ++ | ++ |

Lysis in broth ++ means full lysis (phage sensitivity),
+/− means delayed lysis at low MOI and partial outgrowth (as measured by OD),
− means no lysis (full resistance)
MOI = multiplicity of infection.
N.A. Not tested

### 2.5 Escape Mutant Isolation & Characterization

To assess if phages could bypass the defence systems by incorporating phage-specific mutations, but also to reveal possible triggers for each of the novel phage resistance systems by examining the genes that had to mutate for the phage to escape the defence, as previously described (Stokar-Avihail et al, 2022), phage escape mutants were generated when possible and their full genomes were compared to the wild type phage.

For the isolation of AbiM-escape phage mutants, the lactococcal virulent phages 66901, 62601, P008 & sk1 of the genus Skunavirus (Table 3) were selected for challenge experiments, as those phages were sensitive to AbiM (EOP 10⁻⁷ to 10⁻⁸, Table 5). L. cremoris 3107 and L. lactis IL1403 carrying AbiM, were challenged with high titre (~10⁹ pfu/ml) of the relevant phages 66901, 62601, P008 & sk1. From the escape mutants that were able to overcome AbiM at a rate of approximately 10⁻⁶, three mutants for each phage were randomly selected and were sequenced along with the wild-type phage. Finally, based on the phage genome analysis, among the isolated mutants, eight were non-clonal showing different amino acid substitutions (Table 8). Interestingly, all AbiM-escape mutants shared point mutations in the gene encoding the predicted large subunit of the terminase (Table 8) and more specifically to the ATPase domain. The large terminase is involved in phage DNA packaging (Feiss & Rao, 2012) and is responsible for pumping the DNA into the empty procapsid shell and then for cleaving (endonuclease) when one genome-length of DNA has been packaged (Hilbert et al., 2017).

The seven AbiM-escape mutants were subsequently used to test if they could bypass any of the other novel Abi systems as well as several known Abi systems (e.g. novel Abi systems AbiW, and AbiY and known Abi systems AbiB, AbiJ, AbiP and AbiZ, respectively). Interestingly, all the Abi systems tested were resistant to the AbiM escape mutants, indicating possibly a different activation/target, which is a characteristic of these anti-phage mechanisms.

Strains carrying AbiW and AbiX were challenged with various phages of the genus Skunavirus (e.g. sk1, 66901 & P008), but no escape mutants were found capable of bypassing the newly identified phage defence systems when expressed in the high-copy number vector pNZ44 despite a high phage titre challenge (~10⁹ pfu). Nonetheless, AbiX-escape escape mutants were isolated when L. cremoris NZ9000 bearing abiX was challenged with phage φLL69075. Genome analysis of the AbiX-69075 escape mutants showed that two of the three mutants had a different mutation in the same genomic position of the Sak protein-encoding gene (Table 8). It is known that Sak (sensitivity to Abik), is a recombinase with ssDNA binding properties, which is involved in dsDNA break repair during DNA replication, but how Sak is associated with AbiK is unclear (Bouchard & Moineau, 2004; Dy et al., 2014).

Regarding AbiY, three sk1 escape mutants were isolated. Genome sequencing revealed that they carry the same mutation in the putative DNA polymerase subunit-encoding gene (Table 8). DNA polymerase is the main enzyme responsible for phage DNA replication (Doublié et al., 1998), and is probably associated with either the activation of AbiY or the targeting of this anti-phage mechanism.

TABLE 8
Characteristics of the phage genes of the genus Skunavirus
involved in sensitivity of AbiM, AbiX & AbiZ

| Escape mutant | Number of | | | | |
|---|---|---|---|---|---|
| (Abi.phage.number) | mutations | Gene (locus tag) | Size (aa) | Mutation(s) | Function |
| AbiM.66901.1 | (1) | Terminase | 540 | H204Q | DNA |
| | | large subunit | | | packaging |
| | | (66901_gp03) | | | |
| AbiM.66901.2 | (2) | Terminase | 540 | T58A & | DNA |
| | | large subunit | | S108I | packaging |
| | | (66901_gp03) | | | |
| AbiM.66901.3 | (3) | Terminase | 540 | S108I | DNA |
| | | large subunit | | | packaging |
| | | (66901_gp03) | | | |
| | | Terminase | 174 | E101K | DNA |
| | | small subunit | | | packaging |
| | | (66901_gp01) | | | |
| | | Receptor- | 264 | G78W | Tail and |
| | | binding protein | | | base-plate |
| | | (66901_gp20) | | | morphogenes |
| | | | | | is |
| AbiM.62601.1 | (1) | Terminase | 540 | A132G | DNA |
| | | large subunit | | | packaging |
| | | (62601_gp02) | | | |
| AbiM.62601.2 | (2) | Terminase | 540 | A132G | DNA |
| | | large subunit | | | packaging |
| | | (62601_gp02) | | | |
| | | Terminase | 174 | E101K | |
| | | small subunit | | | |
| | | (62601_gp01) | | | |
| AbiM.P008.1 | (1) | Terminase | 540 | G233V | DNA |
| | | large subunit | | | packaging |
| | | (P008_gp02) | | | |
| AbiM.P008.2 | (1) | Terminase | 540 | H204N | DNA |
| | | large subunit | | | packaging |
| | | (P008_gp02) | | | |
| AbiM.sk1.1 | (1) | Terminase | 540 | G233V | DNA |
| | | large subunit | | | packaging |
| | | (sk1p02) | | | |
| AbiY.69075.1 | (1) | sak protein | 207 | G22D | Recombinase/ |
| | | (69075_40) | | | DNA |
| | | | | | replication |
| AbiY. 69075.2 | (2) | sak protein | 207 | G22A | Recombinase/ |
| | | (69075_40) | | | DNA |
| | | | | | replication |
| AbiZ.sk1.1 | (1) | Putative DNA | 316 | E16K | DNA |
| | | polymerase | | | replication |
| | | subunit | | | |
| | | (sk1p46 & | | | |
| | | p45) | | | |

### 2.6 Phage Resistant Strains Generated by Natural Transfer of the Novel Abi System

Interestingly, at least three of the novel Abi systems (AbiW, AbiX and AbiY) are encoded on pMRC01-like conjugative plasmids (pLL81569D, pLL69075B and pLL66563D, respectively), as shown by BLASTP alignment against the two prevalent lactococcal conjugation systems on pNP40 and pMRC01 (Ortiz Charneco et al., 2021). Plasmid pMRC01 has previously been successfully transferred to over 30 different lactococcal strains, including commercial starter strains (Hickey et al., 2001). Therefore, the novel Abi systems may naturally be transferred to strains of interest by conjugation, to generate non-GMO starter strains with enhanced anti-phage characteristics that can be used in the food industry.

Plasmid pLL69075B was selected as a representative for mating experiments, as this plasmid encodes several additional (predicted) phage-resistance systems including AbiA, AbiZ and AbiX, as well as a restriction modification (RM) system. Given that there was no obvious suitable selection marker for the plasmid, high efficiencies were necessary to screen for a transconjugant among the recipient strains. Indeed, pLL69075B was successfully transferred by conjugation to three strains (L. cremoris NZ9000, L. cremoris MG1614 & LL64983). Although the conjugation efficiency cannot be estimated due to the lack of a selectable phenotype, one to two of fifty randomly plasmid-specific PCR-screened recipient colonies were shown to represent transconjugants. The presence of pLL69075B in L. cremoris MG1614, L. cremoris NZ9000::pJP005 and LL64983 was further verified by using NZ9000/MG1614/LL64983-specific primers (SEQ ID Nos: 33-40).

The transconjugants were subsequently challenged against phages that infect the recipient strain (i.e. 712, p2, sk1, jj50 for L. cremoris NZ9000 & MG1614 and LL64983.1-5 for LL64983). Indeed, the three transconjugants were resistant against all the above-mentioned phages of the genus Skunavirus that infect the strains (Table 9). Therefore, it was confirmed that pLL69075B with its associated anti-phage traits can be transferred by conjugation.

TABLE 9
Obtained phage-resistance of the transconjugants (L. cremoris NZ9000::pJP005::p60975B,
L. cremoris MG1614::pLL69075B & LL64983::pLL69075B) compared to the recipient/wild type
strains (L. cremoris NZ9000::pJP005, L. cremoris MG1614 & LL64983)

| | Strain | | | | | |
|---|---|---|---|---|---|---|
| | L. cremoris | L. cremoris | L. cremoris | L. cremoris | | |
| Phage | NZ9000::pJP005 | NZ9000::pJP005::p60975B | MG1614 | MG1614::pLL69075B | LL64983 | LL64983::pLL69075B |
| sk1 | S | R | S | R | N.A. | N.A. |
| 712 | S | R | S | R | N.A. | N.A. |
| jj50 | S | R | S | R | N.A. | N.A. |
| φLL64983.1 | N.A. | N.A. | N.A. | N.A. | S | R |
| φLL64983.2 | N.A. | N.A. | N.A. | N.A. | S | R |
| φLL64983.3 | N.A. | N.A. | N.A. | N.A. | S | R |
| φLL64983.4 | N.A. | N.A. | N.A. | N.A. | S | R |
| φLL64983.5 | N.A. | N.A. | N.A. | N.A. | S | R |

R, resistant;
S, sensitive (based on spot assays);
N.A.: phage does not infect the relevant strain

### Summary of Performance of Novel Abi Phage-Resistance Mechanisms

The new sequences offer high resistance against phages such as those of the Skunavirus, Ceduovirus, and P335 phage groups and the resistance profile of the novel systems are clearly different compared to known ones tested. Strains with an introduced novel resistance mechanism (GMO) became phage resistant. Importantly the resistance is maintained in liquid cultures, offering utility for lactococcal cultures that are used for dairy fermentation such as milk fermentation. Resistance was observed regardless of the growth phase and is relevant for the conditions usually found in non-sterile dairy production facilities.

Strains encoding the novel Abi systems could directly be employed as starter cultures with improved anti-phage characteristics in dairy fermentations. Additionally, novel Abi systems have been and could be found encoded on pMRC01-like conjugative plasmids, highlighting the potential of these anti-phage activities to be transferred by conjugation to generate robust starter strains with enhanced phage-resistance properties. Conjugation is a non-genetically modified organisms (GMO), horizontal gene transfer (HGT) mechanism that involves natural transfer of genetic material from a donor to a recipient cell via a conjugative apparatus through direct cell-to-cell contact. Since conjugation is regarded a food-grade process, it can therefore be used to generate novel dairy starter cultures which are not considered GMOs and which enjoy enhanced anti-phage characteristics. Indeed, as an example for conjugation, a representative plasmid (LL69075_pB) harboring the novel abix gene was successfully transferred by conjugation to three phage-sensitive lactococcal strains. In addition, the plasmid LL69075_pB contains additional Abi systems (AbiA, AbiZ), showing that Abi systems can be used in combinations, being on one or on alternatives plasmids present within a cell. This conferred phage-resistance against all the phages of the genus Skunavirus that infect these strains.

### Example 3

### Identification of Seven Additional Novel Non-RM Phage-Defense Systems

### 3.1 Identification of Additional Putative Plasmid-Encoded Non-RM Phage Defense Systems

From the initial in silico analysis (as set out in sections 2.1 and 2.2) and the screening of in total 198 candidate systems (as set out in sections 2.3), seven additional non-RM phage defense systems were identified in the initial data set, and these were demonstrated to exert surprising phage resistance-activity when expressed individually in the abovementioned Lactococcus model strains (further described in following examples).

The seven mentioned non-RM phage defense systems are the following:
Abi28: two gene-system consisting of abi28a (SEQ ID NO: 55) and abi28b (SEQ ID NO: 56), representing locus tags LL75972_pC26 and LL75972_pC25, respectively. Translational protein products are Abi28a (SEQ ID NO: 68) and Abi28b (SEQ ID NO: 69).
Abi29: three gene system consisting of abi29a (SEQ ID NO: 57), abi29b (SEQ D NO: 58) and abi29c (SEQ ID NO: 59), representing locus tags LL75843_pB62, LL75843_pB61 and LL75843_pB60, respectively. Translational protein products are Abi29a (SEQ ID NO: 70), Abi29b (SEQ ID NO: 71) and Abi29c (SEQ ID NO: 72).
Abi30: one gene-system consisting of abi30 (SEQ ID NO: 60), representing locus tag LL79472_pA59. Translational protein product is Abi30 (SEQ ID NO: 73).
Abi31: one gene-system consisting of abi31 (SEQ ID NO: 61), representing locus tag LL66563_pC18. Translational protein product is Abi31 (SEQ ID NO: 74).
Abi32: three gene-system consisting of abi32a (SEQ ID NO: 62), abi32b (SEQ ID NO: 63) and abi32c (SEQ ID NO: 64), representing locus tags LL66563_pD51, LL66563_pD50 and LL66563_pD49, respectively. Translational protein products are Abi32a (SEQ ID NO: 75), Abi32b (SEQ ID NO: 76) and Abi32c (SEQ ID NO: 77).
Abi33: two gene-system consisting of abi33a (SEQ ID NO: 65) and abi33b (SEQ ID NO: 66), representing locus tags pLL56542_pE17 and pLL56542_pE16, respectively. Translational protein products are Abi33a (SEQ ID NO: 78) and Abi33b (SEQ ID NO: 79).Abi34: one gene-system consisting of abi34 (SEQ ID NO: 67), representing locus tag LLA22_pB040. Translational protein product is Abi34 (SEQ ID NO: 80).

### 3.2 Anti-Phage Spectrum of the Additionally Identified Phage Defence Systems

Similarly, as in section 2.3, to evaluate the antiphage activity of the novel phage-resistance systems and whether associated with a specific phage (e.g. sk1, bIL66 or c2) or with a whole phage group (i.e. Skunavirus, Ceduovirus, P335 & P087 phage groups), various lactococcal bacteriophages representing the afore-mentioned groups were employed in this study (Table 3). Similar as beforementioned, pNZ44- or pPTPi-derivatives bearing the individual additional seven novel phage defense systems were introduced to L. cremoris NZ9000, L. cremoris 3107 and L. lactis IL1403. These experiments again allowed for assessment of spectrum of anti-phage activity against various Skunavirus (former 936-group) and Ceduovirus (former C2-group) phages and phages belonging to the P335 and P087 groups.

Table 10 summarizes the efficiency of plaquing (EOP) of each employed phage on the lactococcal strain (i.e., L. cremoris NZ9000, L. cremoris 3107, L. lactis IL1403) harbouring each of the novel Abi systems compared to the wild type strain carrying the empty vector (pNZ44 or pPTPi). As the nisin-inducible PnisA promoter requires the NisRK two-component system, which is not present in L. cremoris 3107 or L. lactis IL1403, the pPTPi-derived constructs were only tested in L. cremoris NZ9000. Additionally, some of the systems could not be introduced or were not stable in L. cremoris 3107 or L. lactis IL1403 (Abi30, Abi32), and therefore the corresponding EOP values could not be defined.

Except for Abi28, all other Abi systems provided (varying levels of) resistance against at least one Skunavirus phage, which are the most frequently encountered phages in the dairy industry (Mahony et al., 2012). Abi29 provided over 2 log EOP reduction. Strikingly, Abi30, Abi31, Abi32, Abi33 and Abi34 exhibited high resistance (over four log EOP reduction) against most of the assessed Skunavirus phages in the NZ9000 background (strain). Abi30 also provided an EOP reduction against Skunavirus phages in the IL1403 background. Similarly, for the IL1403 background, Abi34 provided a three log EOP reduction against 3 out of 5 Skunavirus phages, and, in the 3107 background, a limited EOP reduction against a Skunavirus phage.

Against Ceduovirus phages, Abi28, Abi30 and Abi32 showed varying levels of resistance in some cases associated with reduced plaque size.

Abi30 displayed a versatile resistance profile since also an EOP reduction was recorded against a P335 phage in the IL1403 background.

In summary, all tested phage defense systems displayed a level of resistance against one or more relevant phage groups found in infections in the dairy environment.

Table 10
Efficiency of plaquing (EOP) of different phage group representatives
(Skunavirus, Ceduovirus, P335 & P087 phage group) on different lactococcal hosts (NZ9000, 3107,
IL1403) carrying the novel phage-resistance systems Abi28, Abi29, Abi30, Abi31, Abi32, Abi33, and
Abi34. Dark grey highlighted cells with ^{$} in front of value showed considerable plaque size reduction.

| | Skunavirus phages | | | | |
|---|---|---|---|---|---|
| Lactococcal strain | sk1 | 712 | p2 | JJ50 | |
| NZ9000 :: pNZ44/NZ9000 :: pPTPi | 1 | 1 | 1 | 1 | |
| NZ9000 :: pPTPi+Abi28 | ≈1 | ≈1 | ≈1 | ≈1 | |
| NZ9000 :: pPTPi+Abi29 | 2.56 (± | 1.03 (± | 2.02 (± | 0.89 (± | |
| | 1.47) × 10⁻² | 0.58) × | 1.07) × | 0.62) × | |
| | | 10⁻³ | 10⁻³ | 10⁻³ | |
| NZ9000 :: pNZ44+Abi30 | ≤ 1.81 (± | ≤ 2.45 (± | ≤ 9.03 (± | ≤ 4.68 (± | |
| | 1.32) × 10⁻⁹ | 2.21) × | 3.18) × | 1.04) x | |
| | | 10⁻⁷ | 10⁻⁹ | 10⁻⁸ | |
| NZ9000 :: pPTPi+Abi31 | ≤ 2.88 (± | ≤ 6.64 (± | ≤ 2.31 (± | ≤ 0.87 (± | |
| | 0.56) × 10⁻⁸ | 2.13) × | 0.13) × | 0.33) × | |
| | | 10⁻⁷ | 10⁻⁷ | 10⁻⁷ | |
| NZ9000 :: pNZ44+Abi32 | 1.54 (± | 1.03 (± | ≤ 7.47 (± | ≤ 4.19 (± | |
| | 0.07) × 10⁻⁵ | 0.96) × | 1.40) × | 0.35) × | |
| | | 10⁻⁶ | 10⁻⁸ | 10⁻⁸ | |
| NZ9000:pPTPi+Abi33 | ≤ 2.88 (± 0.56) × 10⁻⁸ | ≤ 6.64 (± 2.13) × 10⁻⁷ | 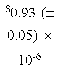 | ≤ 0.87 (± 0.33) × 10⁻⁷ | |
| NZ9000 :: pNZ44+Abi34 | 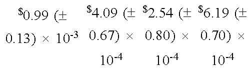 | | | | |
| | Skunavirus phages | | | | |
| Lactococcal strain | 66901 | 62601 | | | |
| 3107 :: pNZ44 | 1 | 1 | | | |
| 3107 :: pNZ44+Abi34 | ≈1 | 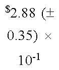 | | | |
| | Skunavirus phages | | | | |
| Lactococcal strain | bIL66 | bIL70 | P008 | P113G | 340 |
| IL1403 :: pNZ44 | 1 | 1 | 1 | 1 | 1 |
| IL1403 :: pNZ44+Abi30 | 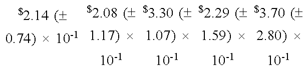 | | | | |
| IL1403 :: pNZ44+Abi34 | 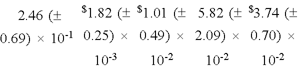 | | | | |
| | P335-phages | | | | |
| Lactococcal strain | TP901-1 | LC3 | Dub35A | 63301 | P335 |
| 3107 :: pNZ44 | 1 | 1 | 1 | 1 | |
| 3107 :: pNZ44+Abi34 | ≈1 | ≈1 | ≈1 | ≈1 | |
| IL1403 :: pNZ44 | | | | | 1 |
| IL1403 :: pNZ44+Abi30 | | | | | 2.80 (± |
| | | | | | 1.30) × |
| | | | | | 10⁻¹ |
| IL1403 :: pNZ44+Abi34 | | | | | ≈1 |
| | Ceduovirus | P087 | | | |
| | phage | phage | | | |
| Lactococcal strain | C2 | P087 | | | |
| NZ9000 :: pNZ44/NZ9000 :: pPTPi | 1 | | | | |
| NZ9000 :: pPTPi+Abi28 | 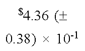 | | | | |
| NZ9000 :: pPTPi+Abi29 | ≈1 | | | | |
| NZ9000 :: pNZ44+Abi30 | 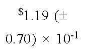 | | | | |
| NZ9000 :: pPTPi+Abi31 | ≈1 | | | | |
| NZ9000 :: pNZ44+Abi32 | ≤ 1.48 (± | | | | |
| | 0.26) × 10⁻⁸ | | | | |
| NZ9000 :: pPTPi+Abi33 | ≈1 | | | | |
| NZ9000 :: pNZ44+Abi34 | ≈1 | | | | |
| 3107 :: pNZ44 | | 1 | | | |
| 3107 :: pNZ44+Abi34 | | ≈1 | | | |

### 3.3 the Newly Identified Lactococcal Antiphage Systems are Active Post Phage DNA Injection

Similar to section 2.4, to examine what stage of the phage cycle the newly identified antiphage systems interfere with, phage adsorption and transduction assays were performed. In FIG. 9 the results of the adsorption and transduction assays are depicted together with several Abi systems (AbiM, AbiW, AbiX, AbiY, AbiZA, Abi27) which were already discussed in section 2.4.

Adsorption assays (FIG. 9A-C) revealed that the assessed systems do not interfere with phage sk1 or c2 adsorption. Similarly, DNA injection does not appear to be noticeably affected by the tested antiphage systems as the observed transduction frequencies are comparable to those obtained for the control strains (FIG. 9D-F). Whereas Abi29, Abi31, Abi32 (both with sk1 and c2), Abi33 and Abi34 showed similar transduction frequencies as the respective control strains, Abi30 was associated with a reduction in transduction frequency. However, this effect is negligible when compared to the observed transduction frequency reduction of a strain expressing the known DNA injection blocking protein Sie2009 (Mahony et al., 2008), whose transduction frequencies were below the detection limit (<1.05×10-7). Therefore, the new antiphage systems do not appear to specifically target phage adsorption or DNA injection steps, indicating that they are active intracellularly at a stage beyond phage DNA injection. This further substantiates the hypothesis the tested anti-phage systems can be categorized as Abi systems.

### 3.4 Lysis in Broth Assays

To assess if the newly identified antiphage systems exhibit the phenotypic characteristics of abortive infection systems, the lysis profiles of L. cremoris NZ9000 and its derivative strains expressing antiphage systems were determined at a low (0.05) and a high (5) multiplicity of infection (MOI) of phage sk1 or c2 (FIG. 10). A phenotype of providing phage resistance at a low MOI, yet entering a bactericidal/bacteriostatic state at a high MOI is consistent with an antiphage system acting through abortive infection (Lopatina et al. 2020, Garb et al. 2022). The same assay for the previous Abi systems is described above. There, systems AbiM, AbiW, AbiX, AbiY, AbiZA, and Abi27 were discussed and growth curves of lactococcal strains expressing these systems can also be seen in FIG. 10.

All additionally tested systems provided phage resistance under low MOI conditions, growing at rates comparable to those of uninfected strains, while corresponding cultures carrying the empty vectors were shown to collapse. However, at a high MOI, all cultures, including those expressing the newly identified defense systems demised or entered a state of bacteriostasis. Therefore, the assessed systems Abi29, Abi30, Abi31, Abi32, Abi33, Abi34 phenotypically all behave like an Abi system. It should be noted that the uninfected control for some strains expressing antiphage systems were shown to grow slower than the uninfected empty vector control, indicating that expression within this experimental design (i.e. plasmid backbone, promoter used) of certain systems comes at a fitness cost (Westra et al., 2015; Bernheim and Sorek 2020).

### Summary of Performance of Additional Novel Abortive Infection Systems to Lactococcus: Abi28. Abi29, Abi30, Abi31, Abi32, Abi33, Abi34

Each of the seven new Abi systems, which DNA sequences are jointly listed by SEQ ID NOs: 55-67 and which protein sequences by SEQ ID NOs: 68-80, offer resistance against phages such as those of the Skunavirus, Ceduovirus, and P335 phage groups. Strains with a recombinantly introduced novel resistance mechanism became phage resistant. The resistance was characterized in all cases to be manifesting intracellularly (post-injection) and showed all the characteristics of an abortive infection type of phage resistance mechanism. Importantly the resistance is maintained in liquid cultures, offering utility for lactococcal cultures that are used for dairy fermentation such as milk fermentation. Resistance was observed regardless of the growth phase and is relevant for the conditions usually found in non-sterile dairy production facilities.

Strains encoding the novel Abi systems naturally could directly be employed as starter cultures with improved anti-phage characteristics in dairy fermentations. Additionally, novel Abi systems have been and could be found encoded on pMRC01-like or other conjugative plasmids, even more on co-mobilizable plasmids, highlighting the potential of these anti-phage activities to be transferred by conjugation to generate robust starter strains with enhanced phage-resistance properties. As stated above as well. conjugation is a non-genetically modified organisms (GMO), horizontal gene transfer (HGT) mechanism that involves natural transfer of genetic material from a donor to a recipient cell via a conjugative apparatus through direct cell-to-cell contact. Since conjugation is regarded a food-grade process, it can therefore be used to generate novel dairy starter cultures which are not considered GMOs and which enjoy enhanced anti-phage characteristics. As stated above, a successful example using conjugation and generating a novel dairy starter culture was shown in section 2.6 and conferred phage-resistance against all the phages of the genus Skunavirus that infect the parent strain previously.

Some phage escape mutants were found in section 2.5 that could reinfect strains with some introduced Abi systems. This is a well-known phenomenon and typical for the continuous evolutionary race-to-arms between lactic acid bacterial host and bacteriophage. Since this patent application describes the extraction and characterization of multiple and diverse Abi resistance mechanisms with each system having another mode of action, a benefit could be imagined of designing multiple dairy starter cultures in a rotation scheme comprising multiple strains enhanced with one or more of the different Abi systems. Since escape mutants arise against a specific Abi system, if a starter in a subsequent rotation comprises another Abi system this could suppress the rising infection levels of arising phage mutants. This would allow for a more sustainable and prolonged stable production in a dairy plant.

### REFERENCES

Ainsworth, S., et al., (2014a). MBio, 5(3), 1-11.
Ainsworth, S., et al., (2014b). FEMS Microbiology Reviews, 38(5), 1066-1088.
Altschul, S. F., et al., (1990). Journal of Molecular Biology, 215(3), 403-410.
Altschul, S. F., et al., (1997). Nucleic Acids Research, 25(17), 3389-3402.
Amitai S, et al., (2009) PLoS Genet 5(3):e1000390.
Stokar-Avihail, Avigail & Fedorenko, Taya & Garb, Jeremy & Leavitt, Azita & Millman, Adi & Shulman, Gabriela & Wojtania, Nicole & Melamed, Sarah & Amitai, Gil & Sorek, Rotem. (2022). Discovery of phage determinants that confer sensitivity to bacterial immune systems.
Bateman, A., et al., (2004). Nucleic Acids Research, 32(DATABASE ISS.), 138-141.
Bebeacua, C., et al., (2013). Journal of Virology, 87(22), 12302-12312.
Bernheim A, Sorek R. The pan-immune system of bacteria: antiviral defence as a community resource. Nat Rev Microbiol. 2020 February; 18(2): 113-119. doi: 10.1038/s41579-019-0278-2.
Bidnenko, E., Chopin, A., Ehrlich, S. D., & Chopin, M. C. (2009). BMC molecular biology, 10(4).
Bidnenko, E., Ehrlich, D., & Chopin, M. C. (1995). Journal of Bacteriology, 177(13), 3824-3829.
Bingham, R., et al., (2000). The Journal of biological chemistry, 275(30), 23219-23226.
Bolotin, A., Wincker, P., et al., (2001). Genome Research, 11(5), 731-753.
Bouchard, J. D., & Moineau, S. (2004). Journal of Bacteriology, 186(11), 3649-3652.
Cavanagh, D., Fitzgerald, G. F., & McAuliffe, O. (2015). Food Microbiology, 47, 45-61.
Chandry, P. S., Davidson, B. E., & Hillier, A. J. (1994). Microbiology, 140(9), 2251-2261.
Chopin, M. C., et al., (2005). Current Opinion in Microbiology, 8(4), 473-479.
Christiansen, B., Johnsen, M. G., Stenby, E., Vogensen, F. K., & Hammer, K. (1994). Journal of Bacteriology, 176(4), 1069-1076.
Crutz-Le Coq, A. M., et al., (2002). Microbiology, 148(4), 985-1001.
Deng, Y. M., et al., (1997). FEMS Microbiology Letters, 146(1), 149-154.
Deveau, H., et al., (2006). Applied and Environmental Microbiology, 72(6), 4338-4346.
Doublié, S., et al., (1998). Nature, 391(6664), 251-258.
Dupont, K., et al., (2005). Journal of Applied Microbiology, 98(4), 1001-1009.
Durmaz, E., & Klaenhammer, T. R. (2007). Journal of bacteriology, 189(4), 1417-1425.
Dy, R. L., et al., (2014). Annual Review of Virology, 1(1), 307-331.
Enright, A. J., Van Dongen, S., & Ouzounis, C. A. (2002). An efficient algorithm for large-scale detection of protein families. Nucleic Acids Research, 30(7), 1575-1584.
Erazo Garzon, A., et al., (2019). Complete Genome Sequence of Lactococcus lactis subsp. cremoris 3107, Host for the Model Lactococcal P335 Bacteriophage TP901-1. 8(3), e01635-18.
FDA, 2010. Generally Recognised as Safe (GRAS) Notifications. FDA.http://www.fda.gov/ANimalVeterinary/Products/AnimalFoodFeeds/GenerallyRecognisedasSafeG RASNotifications/default.htm
Feiss, M., & Rao, V. B. (2012). Advances in Experimental Medicine and Biology, 726, 489-509.
Francia, M. V., Varsaki, A., Garcillan-Barcia, M. P., Latorre, A., Drainas, C., and de la Cruz, F. (2004). A classification scheme for mobilization regions of bacterial plasmids. FEMS Microbiol. Rev. 28, 79-100. doi: 10.1016/j.femsre.2003.09.001
Frickey, T., & Lupas, A. N. (2004). Journal of structural biology, 146(1-2), 2-10.
Garb, J., Lopatina, A., Bernheim, A. et al. Multiple phage resistance systems inhibit infection via SIR2-dependent NAD+ depletion. Nat Microbiol 7, 1849-1856 (2022).
Garneau, J. E., & Moineau, S. (2011). Microbial Cell Factories, 10(SUPPL. 1), 1-10.
Garvey, P., Fitzgerald, G. F., & Hill, C. (1995). Appl Environ Microbiol., 61(12), 4321-8.
Garvey, P., et al., (1996). Applied and Environmental Microbiology, 62(2), 676-679.
Gasson, M. J. (1983). Journal of Bacteriology, 154(1), 1-9.
Gish, W., & States, D. J. (1993). Nature Genetics, 3, 266-272.
Guglielmini et al., (2014). Nucleic Acids Research, 42(9): pp. 5715-5727.
Hanson, P., Whiteheart, S. (2005). Nature Reviews Molecular Cell Biology, 6, 519-529.
Hayes, S., Murphy, J., Mahony, J., Lugli, G. A., Ventura, M., Noben, J. P., Franz, C. M. A. P., Neve, H., Nauta, A., & Van Sinderen, D. (2017). Frontiers in Microbiology, 8.
Hickey, R. M., Twomey, D. P., Paul Ross, R., & Hill, C. (2001). Applied and Environmental Microbiology, 67(6), 2853-2858.
Hilbert B J, et al., Nucleic Acids Res. 2017 Apr. 7; 45(6):3591-3605. PMID: 28082398
Higgins, D. L., et al., (1988). Journal of Bacteriology, 170(8), 3435-3442.
Holo, H., and Nes, I. F. (1989). Appl. Environ. Microbiol. 55, 3119-3123.
Hyatt, D., et al., (2010). BMC Bioinformatics, 11, 1-8.
Josephsen, J., & Vogensen, F. K. (1989). FEMS Microbiology Letters, 59(1-2), 161-166.
Kelleher, P., Mahony, J., Bottacini, F., Lugli, G. A., Ventura, M., and van Sinderen, D. (2019). The Lactococcus lactis Pan-Plasmidome. Front. Microbiol. 10:707. doi: 10.3389/fmicb.2019.00707
Kohler, V., Keller, W., & Grohmann, E. (2019). Frontiers in Microbiology, 10.
Koga, M., Otsuka, Y., Lemire, S., & Yonesaki, T. (2011). Genetics, 187(1), 123-130.
Krogh, A., et al., (2001). Journal of Molecular Biology, 305(3), 567-580.
Labrie, S. J., Samson, J. E., & Moineau, S. (2010). Nature Reviews Microbiology, 8(5), 317-327.
Landete, J. M., et al., (2014). Journal of Microbiological Methods, 105, 130-133.
Lau, R. K., et al., (2020). Molecular cell, 77(4), 723-733.e6.
Lillehaug, D. (1997). Journal of Applied Microbiology, 83(1), 85-90.
Lillehaug, D., Lindqvist, B. H., & Birkeland, N. K. (1991). Applied and Environmental Microbiology, 57(11), 3206-3211.
Linares, D. M., Kok, J., & Poolman, B. (2010). Genome sequences of Lactococcus lactis MG1363 (revised) and NZ9000 and comparative physiological studies. Journal of bacteriology, 192(21), 5806-5812.
Loof, M., Lembke, J., & Teuber, M. (1983). Systematic and Applied Microbiology, 4(3), 413-423.
Lopatina, A., Tal, N., & Sorek, R. (2020). Annual review of virology, 7(1), 371-384.
Lugli, G. A., Milani, C., Mancabelli, L., Van Sinderen, D., & Ventura, M. (2016). MEGAnnotator: A user-friendly pipeline for microbial genomes assembly and annotation. FEMS Microbiology Letters, 363(7), 1-8.
Mahony, J., et al., (2013). Applied and Environmental Microbiology, 79(14), 4385-4392.
Mahony, J., McDonnell, B., Casey, E., & Van Sinderen, D. (2016). Annual Review of Food Science and Technology, 7(1), 267-285.
Mahony, J., McGrath, S., Fitzgerald, G. F., & Van Sinderen, D. (2008). Applied and Environmental Microbiology, 74(20), 6206-6215.
Mahony, J., Murphy, J., & Van Sinderen, D. (2012). Frontiers in Microbiology, 3, 1-9.
Mahony, J., et al., (2017). BMC genomics, 18(1), 146.
Makarova, K. S., et al. (2011). Journal of Bacteriology, 193(21), 6039-6056.
Makarova, K., et al., (2006). PNAS, 103(42), 15611-15616.
Marchler-Bauer, A., et al., (2011). Nucleic Acids Research, 39(SUPPL. 1), 225-229.
McGrath, S., Fitzgerald, G. F., & Van Sinderen, D. (2001). Applied and Environmental Microbiology, 67(2), 608-616.
Mills, S., Mcauliffe, O. E., Coffey, A., Fitzgerald, G. F., & Ross, R. P. (2006). FEMS Microbiology Reviews, 30(2), 243-273.
Mills, S., et al., (2010). International Journal of Dairy Technology, 63(2), 149-170.
Mistry, J., Chuguransky, S., Williams, L., Qureshi, M., Salazar, G. A., Sonnhammer, E. L. L.,
Mulder J, Wels M, Kuipers O P, Kleerebezem M, Bron P A 2017. Unleashing Natural Competence in Lactococcus lactis by Induction of the Competence Regulator ComX. Appl Environ Microbiol 83: e01320-17.
Tosatto, S. C. E., Paladin, L., Raj, S., Richardson, L. J., Finn, R. D., & Bateman, A. (2021). Pfam: The protein families database in 2021. Nucleic Acids Research, 49(D1), D412-D419.
O'Brien, F. G., Yui Eto, K., Murphy, R. J., Fairhurst, H. M., Coombs, G. W., Grubb, W. B., et al. (2015). Origin-of-transfer sequences facilitate mobilisation of non-conjugative antimicrobial-resistance plasmids in Staphylococcus aureus. Nucleic Acids Res. 43, 7971-7983. doi: 10.1093/nar/gkv755
O'Driscoll, J., Glynn, F., Cahalane, O., O'Connell-Motherway, M., Fitzgerald, G. F., & Van Sinderen, D. (2004). Applied and Environmental Microbiology, 70(9), 5546-5556.
Oliveira, J., et al., (2018). Journal of Dairy Science, 101(1), 96-105.
Oliveira, J., Mahony, J., Lugli, G. A., Hanemaaijer, L., Kouwen, T., Ventura, M., & van Sinderen, D. (2016). Genome Announcements, 4(6), 4-5.
Ortiz Charneco, G., et al., (2021). Frontiers in Microbiology, 12.
Otsuka, Y., & Yonesaki, T. (2012). Molecular microbiology, 83(4), 669-681.
Pedersen, M. B., et al., (2005). FEMS Microbiology Reviews, 29(3), 611-624.
Rincé, A., et al., (2000). FEMS Microbiology Letters, 182(1), 185-191.
Ross, R. P., et al., (2000). Trends in Food Science and Technology, 11(3), 96-104.
Rousset, F., et al., (2022). Cell Host & Microbe. 30(5), 740-753.e5.
Sayers, E. W., et al., (2021). Nucleic Acids Research, 49(D1), D10-D17.
Schmitt, C. K., & Molineux, I. J. (1991). Journal of bacteriology, 173(4), 1536-1543.
Snyder L. (1995). Molecular microbiology, 15(3), 415-420.
Söding, J., Biegert, A., & Lupas, A. N. (2005). Nucleic Acids Research, 33(SUPPL. 2), 244-248.
Van Pijkeren, J. P., & Britton, R. A. (2012). Nucleic Acids Research, 40(10), 1-13.
Villion, M., et al. (2009). Virology, 388(1), 49-56.
Westra E R, van Houte S, Oyesiku-Blakemore S, Makin B, Broniewski J M, Best A, Bondy-Denomy J, Davidson A, Boots M, Buckling A. Parasite Exposure Drives Selective Evolution of Constitutive versus Inducible Defense. Curr Biol. 2015 Apr. 20; 25(8):1043-9.
Wouters, J. T. M., et al., (2002). International Dairy Journal, 12(2-3), 91-109.
Zrelovs, N., Dislers, A., & Kazaks, A. (2021). Microorganisms, 9(7).

1.-28. (canceled)

29. A method of producing a Lactococcus strain with improved phage resistance, said method comprising introducing a nucleic acid construct encoding an Abortive Infection defensive (Abi) system to a Lactococcus, said nucleic acid construct comprising a first sequence that has at least 80% sequence identity to any one of SEQ ID NO: 60, 1-8, 9-10, and/or 55-59 and/or 61-67, or a plasmid comprising said nucleic acid construct.

30. The method of claim 29, wherein the first sequence is followed by a second sequence that has at least 80% sequence identity to any one of SEQ ID NOs: 1-8 and/or 55-67, or wherein the second sequence encodes a polypeptide with an amino acid sequence that has at least 80% sequence identity to the amino acid sequence as set forth in any one of SEQ ID NOs: 21-28 and/or 68-80, and wherein, optionally, the first and the second sequence have at least 80% sequence identity to SEQ ID NOs: 5 and 6, 7, and 8, 55 and 56, or 65 and 66, respectively, or wherein the first and second sequence optionally encode a polypeptide with an amino acid sequence that has at least 80% sequence identity to the amino acid sequence as set forth in SEQ ID NOs: 25 and 26, 27 and 28, 68 and 69, or 78 and 79, respectively.

31. The method of claim 29, wherein the nucleic acid construct comprises a first and a second sequence, wherein the first and the second sequence have at least 80% sequence identity with SEQ ID NOs: 5 and 6, 7 and 8, 55 and 56, or 65 and 66, respectively or wherein the first and second sequence encode a polypeptide with an amino acid sequence that has at least 80% sequence identity to the amino acid sequence as set forth in SEQ ID NOs: 25 and 26, 27 and 28, 68 and 69, or 78 and 79, respectively.

32. The method of claim 29, wherein the first and second sequences are followed by a third sequence that has at least 80% sequence identity to any one of SEQ ID NOs: 1-8 and/or 55-67, wherein, optionally, the first, second and third sequence have at least 80% sequence identity with SEQ ID NOs: 57, 58 and 59, or 62, 63 and 64, respectively or wherein, optionally, the first second and third sequence encode a polypeptide with an amino acid sequence that has at least 80% sequence identity to the amino acid sequence as set forth in SEQ ID NOs: 70, 71 and 72, or 75, 76 and 77, respectively.

33. The method of claim 29, wherein the nucleic acid construct comprises a first, second and third sequence, wherein the first, second and third sequence have at least 80% sequence identity with SEQ ID NOs: 57, 58 and 59, and 62, 63 and 64, respectively or wherein the first second and third sequence encode a polypeptide with an amino acid sequence that has at least 80% sequence identity to the amino acid sequence as set forth in SEQ ID NOs: 70, 71 and 72, or 75, 76 and 77, respectively.

34. The method of claim 29 wherein said Lactococcus strain encodes an Abortive Infection defensive (Abi) system.

35. The method of claim 34, wherein the production of the Lactococcus strain comprises one or more steps selected from conjugation, transformation, mutagenesis and/or genome editing and subsequent selection for improved phage resistance.

36. A method of producing a Lactococcus strain with improved phage resistance, said method comprising providing a host cell comprising a nucleic acid construct encoding an Abortive Infection defensive (Abi) system, said nucleic acid construct comprising a first sequence that has at least 80% sequence identity to any one of SEQ ID NO: 60, 1-8, 9-10, and/or 55-59 and/or 61-67 or a plasmid comprising said nucleic acid construct.

37. A method for the production of a Lactococcus strain with improved phage resistance, the method comprising:
(i) providing a host cell as donor strain, said host cell comprising a nucleic acid construct encoding an Abortive Infection defensive (Abi) system, said construct comprising a first sequence being involved that has at least 80% sequence identity to any one of SEQ ID NO: 60, 1-8, 9-10, and/or 55-59 and/or 61-67 or a plasmid comprising said nucleic acid construct;
(ii) providing a recipient Lactococcus strain;
(iii) contacting said donor strain of step (i) with the recipient Lactococcus strain to obtain a transconjugant
(iv) selecting for a transconjugant comprising the conjugative plasmid to identify a strain with improved phage resistance; and optionally
(v) isolating the strain with improved phage resistance.

38. A method for the production of a Lactococcus strain with improved phage resistance, the method comprising:
(i) providing a recipient Lactococcus strain;
(ii) contacting the recipient Lactococcus strain with a donor Lactococcus strain, wherein the donor strain comprises a conjugative plasmid encoding an Abortive Infection defensive (Abi) system that comprises:
a nucleotide sequence that has at least 80% sequence identity to any one of SEQ ID NOs: 60, 1-8 and/or 55-59 and/or 61-67; or
a nucleotide sequence that has at least 80% sequence identity to SEQ ID NO: 9 or SEQ ID NO: 10, or
a nucleotide sequence that encodes a polypeptide with an amino acid sequence that has at least 80% sequence identity to the amino acid sequence as set forth in any one of SEQ ID NOs: 73, 21-28 and/or 68-72, and/or 74 80,
to obtain a transconjugant;
(iii) selecting for a transconjugant comprising the conjugative plasmid to identify a strain with improved phage resistance; and optionally
(iv) isolating the strain with improved phage resistance.

39. A method for the production of a starter culture for the production of a fermented food product, said starter culture comprising one or more lactic acid bacteria strain(s), wherein at least one of said strain(s) is a Lactococcus strain with improved phage resistance comprising a nucleic acid construct encoding an Abortive Infection defensive (Abi) system, said construct comprising a first sequence being involved that has at least 80% sequence identity to any one of SEQ ID NO: 60, 1-8, 9-10, and/or 55-59 and/or 61-67 or a plasmid comprising said nucleic acid construct.

40. A starter culture obtainable by the method of claim 39, said starter culture being used for the production of a fermented food product, said starter culture being in the form of frozen, lyophilized, spray-dried, vacuum-dried, air dried, tray dried, or in liquid form, said starter culture optionally further comprising:
(i) an excipient selected from the group consisting of a cryoprotectant, a lyoprotectant, an antioxidant, a nutrient, and mixtures thereof; and/or
(ii) a further strain selected from Lactococcus, Lactobacillus and/or Streptococcus, optionally a further strain selected from Lactococcus, Lactobacillus helveticus and/or Streptococcus thermophilus, optionally a further strain selected from Lactococcus, Lactococcus helveticus, Streptococcus thermophilus, or both a strain of Lactobacillus helveticus and a Streptococcus thermophilus.

41. A method for the production of a fermented food product, the method comprising the step of fermenting a substrate with an isolated strain obtainable by the method according to claim 37, particularly wherein the method is performed in a non-sterile environment.

42. A method for the detection and/or isolation of a constructed or naturally occurring strain comprising a nucleic acid construct encoding an Abortive Infection defensive (Abi) system, said construct comprising a first sequence being involved that has at least 80% sequence identity to any one of SEQ ID NO: 60, 1-8, 9-10, and/or 55-59 and/or 61-67 or a plasmid comprising said nucleic acid construct, said method comprising:
(i) providing a sample comprising or suspected to comprise a Lactococcus strain comprising a nucleic acid construct encoding an Abortive Infection defensive (Abi) system, said construct comprising a first sequence being involved that has at least 80% sequence identity to any one of SEQ ID NO: 60, 1-8, 9-10, and/or 55-59 and/or 61-67 or a plasmid comprising said nucleic acid construct;
(ii) culturing the sample on a suitable substrate optionally an agar plate,
(iii) detecting whether said nucleic acid construct as defined in step (i) is present in the cultured sample, optionally by colony polymerase chain reaction (PCR); and optionally,
(iv) isolating a detected strain from the culture.


SEQUENCE LISTING (92 sequences)

SEQ ID NO: 1 - DNA, 912 bp, Lactococcus lactis
  source 1..912: mol_type = other DNA
atggaaatattaaaaaaaagaattaataatgttgaaaaatatctatcagacatttcagacaaccaaaatttttttatcggaattaaaattgaaccgaacaaaatccaaaaaatactatcagaaaaatttaacaaaactgacgaaaatggattgatgtttattccaaaaccctataatggaataatggctcaacgtaataccgtgggagagtttttatctgacaaaagtaaacccaaagaaactgcctatcgcgctcagagttggtctctaaaagattggggaggaaatattcattctggaactagtgaagtaccttataaaagatatcctaaaatttttatagatcccttcggcctcaacttcattttacttaatgataaacagtatttagtaattaataagcaattcaataaaaaaaaatctgaatatgacaaaataaaatcagccatcaatctcacattagaattattcaataaagctgaaacttttatactagacgtagacacagatatgctaaaaccagttagaaaaagtgttccttgggaaattctcccccaaggggttagaatttggaatgcttttaaagatgtagctaattctaatcaagtatcaaaaagtgctaaaatattaatcgaagaaagatttaaatatttagaagaattcaagccagatgttgagtatcaaggtactgctggatattcaggatatatagcttttgctttttcaagtaaaaatatctatgttctagattctatcttgtatggaaatgctacgtatatttttggtggtaattgggaggaaatatctcaattaactaaaaaacaaataattgatgataatttatataaatatagaattatacatgataaaaattggaaaattgaaataggaaatattcttaattctatctag

SEQ ID NO: 2 - DNA, 951 bp, Lactococcus lactis
  source 1..951: mol_type = other DNA
atgaaatttgaagattataaattagaaattcctgatagagatcctgtttctgaagagaactttgatatagagacctggcgagaagaaaatccgattgattacttaaaagctctacatcttattgctaagcagcattctatacaaaaatgtgcattcgacactatatttacagtgctaaagctctatcttccaaatatattatataaatattactcgcttaatgaaaatatggatttaaataatcagaagtttgaaactataaaaaatcaaaagatatatactgctgaaacaaaagcattaaatgatccttttgaaggcaaggcgttttattatgacccaaataaattaatgaaatttaaacctttggataaagttgatggtcatttaattgatgacttttccgattatgtacgtattgcatctctaaccgggaatggtaccgattcaatgccaatgtgggctcattatgcaaataatcataaaggattctgtgtttcttacgatttaagtgaaaatataaacttagaactacgctctgtaacgtttcctgtgcaatatacgaacaagagaattgatattactagtattatggaacatacagttataaatattttaaatagaatatcttcatcaacagtaaaccaaattatgcttgatgatatgttaatgatctatgctccaatatttctatcaaatataaaacatgatttttggaattatgagaacgaattcagaattacagtaggtaatgataaaaacatgaaatatataaatgcaattcctaaagcattttttttaggtattaaatgcgaaaaaaaaaatcgcgagagattgcaagaaatagctgtcgatcttaaaattccagtatatgaaatgttttatgatgatatggatgataattttaagttaagcaaaagacaaatttatccactattggactttaagttataa

SEQ ID NO: 3 - DNA, 543 bp, Lactococcus lactis
  source 1..543: mol_type = other DNA
atggaattaacctttggtttaaaagaacgtttaatcttagctaatcagtatgaaattttatcaaaaatttcagaggacaaatctgatgctagctacttcgagaatttagcagagatttttaggtctggttatactagaaattacgaggaagcttttaaaggtttcgaagaaaatgagcttagccaagaagattgtaagtttgttgtagatgttttgaatttatatcgtgaattatataagacgtgggaagagttagaagaagttcatgagatcattgaagaagacgatgtattatttcagggatttgatggaaatgaagaagatcaatattatgcttattgcaaattcttagttgaagatcaagggttgtggggagagctaaaagatcttatgaaagaaggaaaaattaacttgaattcccaccggccaatgagagataaatatgaaaattatttattaaaacttaaagaaattcaaagccgaaaagaatatgatgtctcagaatatttgacacttgaagaaattgaatacattttgaagtaa

SEQ ID NO: 4 - DNA, 1179 bp, Lactococcus lactis
  source 1..1179: mol_type = other DNA
atgaatggattatttggtgataatatattctttctatttctttttacttgtataacaataatgaactattccagaatgtctgaaactcaaaaaatgacattattttatttaatgacatttggtttagtcctattaaaaattataagcttcactgaaagtatattatatttatttattattttatttctctatcttgaatttttaacagaggattctaaaaaaaataaatatattacaaaacttagatacaagattctagacttcttgtttttgattttttttcagtatcaattcacactgttgttaatttcttttatctttgctagtacaatggttcaaactaatatttcaaaaattagtttatttccttttactctcagtaattccttctttcaattaatttcgataatattttttttcatagcaatatttaaaatgtctcaagaaaagtttaaagtaaagagtttatcttccttaattaaggatgtgtttcaaccctcgataaatgaaagtcatttagacccttacacaagatactttccaattttggcagacatggaagataaaacctatctttcaagaaaaaaaaacagccattgtttttcattcgaaatactgttatctaaaatgaagactttttatcggtcaaaaaagaatataggattctcagagacactcaaaatatataataacattccacaaattattcgtggttattcaactatagaaatgcagctagtaaggagcatttgtattgagtcaggatataataagctactctcaagaaagatatttgaaattctgtatacaaaaattatttttaatagtatgcaggattattataaaaaaaataactatgtaaataacgagcgattcagagagtacttattgataatctacatttcgaatgtcaggactaaaatcaatggaactatttactcaaatatgaccaaatatctgggagaggatgtttctaattggacaaaaaacaaattctacattgcttgcttaggattgccttatcaaacattcacgaaacattatatttttggtacgcatcaaaatatcattattaaatattcactggatactaagtttatagagacagagttggaaaggataggaaatattgagcaaaaatattacggcgcagataaggaccacaacaccctaatataa

SEQ ID NO: 5 - DNA, 1362 bp, Lactococcus lactis
  source 1..1362: mol_type = other DNA
atgcaattaggagcaaaaatgaaattagaaaacctattcatagaaaaattacatggtatttataattatgatgttgattttaatgaagatattacttttttatacggaagtaatggatgtggaaaaacaacaatactaaatatagttaccagcattgtaactggaaaaatatatgaattatttttttatgactttaagattattaaattgaagtattccaacaacgataatgttgatgttattgaaataaaaagaaagcaagatgataaaaatagattaaaaataatttttgagggtagaaatgaaactatatcaaaattagataatttgaattttcaaagttcaagaagaaagatattattaaaggaatattttcatgaaaataatatattaaaggatatttctcgactatttaactatgtttatctacctttaagtagatcagcttatgttgatcctataaggtcatccagaaaaaacttttggatagaggaagaggaaatagaagaagagttatatgtaacaaaagatgcctcattaacacgagtacaagaactcattgttaataaaattttaactataaataatgaagttaaccggttaaataataactttagagataaaatcttgaaaacatcaattaatatttattcagaaaagaaaaattttagtgatatgataaatgaaattgagaattcaaatgaagaaaaaatactagaaatacaagaatcatacttaaatataatgacaaatttaaaattaattaatgaaagagaaatagaaaaatacaataatttcttttctagttacataaaaaaatatgaacaaataactgatgaaaatgatgggaaagtaaaacattttccaattaatttagtattacaatattcagagatatctaaaataagaaaagttgttgaactagctcaacagatggaaaaagataaaaagcaagcagaaaaacctttggaattattctgtagtacattaaatagttttttaagcagaccaggtgatattgatagtaaaaatataataattgaaactattggtcaacctggagtttattttaagataccgaataatcaaaacaaattaagtgtccaaaaattatcatcaggagaaaaacagttgattatattttttgctaatttgatcttcggagttaatgatagaaaaaatggtatttttatagttgatgagcctgaattatcactacatctaacttggcaaaaattatttatagagcaaacattaaaaattaataataatattcaattgatatttgcaacacattcaccagaaattgtaggtagacatagagataaaatgtataagttagaaaaagtgttaggagaatag

SEQ ID NO: 6 - DNA, 843 bp, Lactococcus lactis
  source 1..843: mol_type = other DNA
atggaagagttagcatattcagatgaaggagttatttccagatattttttctttgaaggcgtaaataaactggatgtttttgttgaagatgtggggaaagaatacgagtatgaaactatattaaaaaaactatttggagagaaatataaaatatcggaaatctttcctttgggtgggaaaaacaatgttattaaaaccttggatgaatttattgaaaatggaaaaattgatagtgaaattcctaaaatattcattgtagatggtgattttgataaatatttagataatattgaagtcaaagaacatcctaatttggtttatcttgaaaaatataatatagaaaattattacgttgataaaaaagccacagtatcttttatgaaggggaaattagaaaaattagataaaaaagtagaagaaataattaaatttgaagaatggttagaagaaacagtaaatgatttaaaacctatttttatcctgtattgtatcattcaagaaaatgaattaggaatacctaatgtaggagaatcggagtataggatttttgaagatttatcaggtaaggtaatatctcaggcatatgattcattgaagttacaagttgaaaaagaattacccaatgtttcagagttagaacgaataatggaagaaaaatatctgaaagaaaattctgatttttttgatttaatttgtggaaagcatttattatgtagtttatttttacatttaaaatcctgtggaaagggattcagaagagctgatttagaatggcatttgaagcaaaatattgatatttcaaaactagattttttaaaacaaagagtagaaactttagtttag

SEQ ID NO: 7 - DNA, 573 bp, Lactococcus lactis
  source 1..573: mol_type = other DNA
atggaaaataaaaaaaaatatctttttaaagaaatacagacttttttagtgaatgttggttggacacataaaatacaaatttgccaggttgatgtatatatggagctttcaaaaaatttaaaactggctaaaattattttgacatctttaacttctgctggtttggggagttttattttgaagattttcccaagtattcaggacatagccatgactattacatttgcaatatcgcttgctacaacaattgttatagctttagataaggaaaaagattataagaaattagctgaacagaataaagtggctgcagataaatacttagaaatgcgtaataaagctatgtcgcttctttatagaatgaaatatgaagaaggtattactacaattgaaaatgaatttttagaattgaaagaacttagaaatgaggcagatccaggtttaccacatacatcttatcaagctgttgaactagcaagtaaaaaattgaaagaaaataaagataatgattacactgaggattacaaattatttattcctaaagatttattagacttagaaggtgagatatga

SEQ ID NO: 8 - DNA, 1227 bp, Lactococcus lactis
  source 1..1227: mol_type = other DNA
atgattattgactatgcaaaaactttagaacgttcagaagaggaaattataaaatgggaaaatagaattattaagattaataataagctatgtgataaatattacctagaaagagacaattactctatacttgttggatcagttggaagaggaacagcaattacgaaaactagtgattatgacgtgctatttaaacttcctcagtcgctttacacaaaatttgataaatatgaatcaaacggtcaatctcagttacttcaagaaattaaaaatataatcaaagaattacatcctaaaacaaaaatgaagggagatggacaagtagtttcaattgaatataatgatggggtaattgaacttgtcccagcatttgaacagagtgatggtaatttcaaatatcctgatagtaacaatgggggatgttggaaggtaacaaaaccacttccagagattgaaggctcagtaaaaaaatctaaaaatacttctaaaacttttaattacttgtgctttctgatgcgacaatggaaaaatcataatggcttttcttttaaaggattgcttattgacacattagtgtctgagtatcttgaagatgaaagttacaaaaataaaacagagttacaattattagaaggtttttttggtcaattatctaaagaaaataaagagcaatcatattggtatgccttagggagtaatcaaaaaatctataatgacgataatggtaaatttgtaaccaaagctaaaaaagcattaaaaaaatttgatgaggctacggaagaatcaattttaaaagatttgtttggctataaacaatcagaacaacaggctaccaatgaagaattcattgagaataaatttattgtagatattcgatttaaccttaagattgattgtgatgttatacaaaatggttttagaggtatgaaacttactgaatatatacgaagtaaatttagactaggacgtggaaaaacattgaatttttctgtgattaatagcaatattccctcttatctaccagtaaattattattggaaagttagaaatgttggtagagaggctttaggtaaagagagaggccaaattttcttgggttcgaaaaaactaatcgagcatacaagctttaatgggaaccattatgttgaatgctatgcaatatctgatgggattgtgattgctcgggataagattaaagtacctattgatgtattgcatgggagatag

SEQ ID NO: 9 - DNA, 2205 bp, Lactococcus lactis
  source 1..2205: mol_type = other DNA
atgcaattaggagcaaaaatgaaattagaaaacctattcatagaaaaattacatggtatttataattatgatgttgattttaatgaagatattacttttttatacggaagtaatggatgtggaaaaacaacaatactaaatatagttaccagcattgtaactggaaaaatatatgaattatttttttatgactttaagattattaaattgaagtattccaacaacgataatgttgatgttattgaaataaaaagaaagcaagatgataaaaatagattaaaaataatttttgagggtagaaatgaaactatatcaaaattagataatttgaattttcaaagttcaagaagaaagatattattaaaggaatattttcatgaaaataatatattaaaggatatttctcgactatttaactatgtttatctacctttaagtagatcagcttatgttgatcctataaggtcatccagaaaaaacttttggatagaggaagaggaaatagaagaagagttatatgtaacaaaagatgcctcattaacacgagtacaagaactcattgttaataaaattttaactataaataatgaagttaaccggttaaataataactttagagataaaatcttgaaaacatcaattaatatttattcagaaaagaaaaattttagtgatatgataaatgaaattgagaattcaaatgaagaaaaaatactagaaatacaagaatcatacttaaatataatgacaaatttaaaattaattaatgaaagagaaatagaaaaatacaataatttcttttctagttacataaaaaaatatgaacaaataactgatgaaaatgatgggaaagtaaaacattttccaattaatttagtattacaatattcagagatatctaaaataagaaaagttgttgaactagctcaacagatggaaaaagataaaaagcaagcagaaaaacctttggaattattctgtagtacattaaatagttttttaagcagaccaggtgatattgatagtaaaaatataataattgaaactattggtcaacctggagtttattttaagataccgaataatcaaaacaaattaagtgtccaaaaattatcatcaggagaaaaacagttgattatattttttgctaatttgatcttcggagttaatgatagaaaaaatggtatttttatagttgatgagcctgaattatcactacatctaacttggcaaaaattatttatagagcaaacattaaaaattaataataatattcaattgatatttgcaacacattcaccagaaattgtaggtagacatagagataaaatgtataagttagaaaaagtgttaggagaatagatggaagagttagcatattcagatgaaggagttatttccagatattttttctttgaaggcgtaaataaactggatgtttttgttgaagatgtggggaaagaatacgagtatgaaactatattaaaaaaactatttggagagaaatataaaatatcggaaatctttcctttgggtgggaaaaacaatgttattaaaaccttggatgaatttattgaaaatggaaaaattgatagtgaaattcctaaaatattcattgtagatggtgattttgataaatatttagataatattgaagtcaaagaacatcctaatttggtttatcttgaaaaatataatatagaaaattattacgttgataaaaaagccacagtatcttttatgaaggggaaattagaaaaattagataaaaaagtagaagaaataattaaatttgaagaatggttagaagaaacagtaaatgatttaaaacctatttttatcctgtattgtatcattcaagaaaatgaattaggaatacctaatgtaggagaatcggagtataggatttttgaagatttatcaggtaaggtaatatctcaggcatatgattcattgaagttacaagttgaaaaagaattacccaatgtttcagagttagaacgaataatggaagaaaaatatctgaaagaaaattctgatttttttgatttaatttgtggaaagcatttattatgtagtttatttttacatttaaaatcctgtggaaagggattcagaagagctgatttagaatggcatttgaagcaaaatattgatatttcaaaactagattttttaaaacaaagagtagaaactttagtttag

SEQ ID NO: 10 - DNA, 1800 bp, Lactococcus lactis
  source 1..1800: mol_type = other DNA
atggaaaataaaaaaaaatatctttttaaagaaatacagacttttttagtgaatgttggttggacacataaaatacaaatttgccaggttgatgtatatatggagctttcaaaaaatttaaaactggctaaaattattttgacatctttaacttctgctggtttggggagttttattttgaagattttcccaagtattcaggacatagccatgactattacatttgcaatatcgcttgctacaacaattgttatagctttagataaggaaaaagattataagaaattagctgaacagaataaagtggctgcagataaatacttagaaatgcgtaataaagctatgtcgcttctttatagaatgaaatatgaagaaggtattactacaattgaaaatgaatttttagaattgaaagaacttagaaatgaggcagatccaggtttaccacatacatcttatcaagctgttgaactagcaagtaaaaaattgaaagaaaataaagataatgattacactgaggattacaaattatttattcctaaagatttattagacttagaaggtgagatatgaatgattattgactatgcaaaaactttagaacgttcagaagaggaaattataaaatgggaaaatagaattattaagattaataataagctatgtgataaatattacctagaaagagacaattactctatacttgttggatcagttggaagaggaacagcaattacgaaaactagtgattatgacgtgctatttaaacttcctcagtcgctttacacaaaatttgataaatatgaatcaaacggtcaatctcagttacttcaagaaattaaaaatataatcaaagaattacatcctaaaacaaaaatgaagggagatggacaagtagtttcaattgaatataatgatggggtaattgaacttgtcccagcatttgaacagagtgatggtaatttcaaatatcctgatagtaacaatgggggatgttggaaggtaacaaaaccacttccagagattgaaggctcagtaaaaaaatctaaaaatacttctaaaacttttaattacttgtgctttctgatgcgacaatggaaaaatcataatggcttttcttttaaaggattgcttattgacacattagtgtctgagtatcttgaagatgaaagttacaaaaataaaacagagttacaattattagaaggtttttttggtcaattatctaaagaaaataaagagcaatcatattggtatgccttagggagtaatcaaaaaatctataatgacgataatggtaaatttgtaaccaaagctaaaaaagcattaaaaaaatttgatgaggctacggaagaatcaattttaaaagatttgtttggctataaacaatcagaacaacaggctaccaatgaagaattcattgagaataaatttattgtagatattcgatttaaccttaagattgattgtgatgttatacaaaatggttttagaggtatgaaacttactgaatatatacgaagtaaatttagactaggacgtggaaaaacattgaatttttctgtgattaatagcaatattccctcttatctaccagtaaattattattggaaagttagaaatgttggtagagaggctttaggtaaagagagaggccaaattttcttgggttcgaaaaaactaatcgagcatacaagctttaatgggaaccattatgttgaatgctatgcaatatctgatgggattgtgattgctcgggataagattaaagtacctattgatgtattgcatgggagatag

SEQ ID NO: 11 - RNA, 912 nt, Lactococcus lactis
  source 1..912: mol_type = transcribed RNA
atggaaatattaaaaaaaagaattaataatgttgaaaaatatctatcagacatttcagacaaccaaaatttttttatcggaattaaaattgaaccgaacaaaatccaaaaaatactatcagaaaaatttaacaaaactgacgaaaatggattgatgtttattccaaaaccctataatggaataatggctcaacgtaataccgtgggagagtttttatctgacaaaagtaaacccaaagaaactgcctatcgcgctcagagttggtctctaaaagattggggaggaaatattcattctggaactagtgaagtaccttataaaagatatcctaaaatttttatagatcccttcggcctcaacttcattttacttaatgataaacagtatttagtaattaataagcaattcaataaaaaaaaatctgaatatgacaaaataaaatcagccatcaatctcacattagaattattcaataaagctgaaacttttatactagacgtagacacagatatgctaaaaccagttagaaaaagtgttccttgggaaattctcccccaaggggttagaatttggaatgcttttaaagatgtagctaattctaatcaagtatcaaaaagtgctaaaatattaatcgaagaaagatttaaatatttagaagaattcaagccagatgttgagtatcaaggtactgctggatattcaggatatatagcttttgctttttcaagtaaaaatatctatgttctagattctatcttgtatggaaatgctacgtatatttttggtggtaattgggaggaaatatctcaattaactaaaaaacaaataattgatgataatttatataaatatagaattatacatgataaaaattggaaaattgaaataggaaatattcttaattctatctag

SEQ ID NO: 12 - RNA, 951 nt, Lactococcus lactis
  source 1..951: mol_type = transcribed RNA
atgaaatttgaagattataaattagaaattcctgatagagatcctgtttctgaagagaactttgatatagagacctggcgagaagaaaatccgattgattacttaaaagctctacatcttattgctaagcagcattctatacaaaaatgtgcattcgacactatatttacagtgctaaagctctatcttccaaatatattatataaatattactcgcttaatgaaaatatggatttaaataatcagaagtttgaaactataaaaaatcaaaagatatatactgctgaaacaaaagcattaaatgatccttttgaaggcaaggcgttttattatgacccaaataaattaatgaaatttaaacctttggataaagttgatggtcatttaattgatgacttttccgattatgtacgtattgcatctctaaccgggaatggtaccgattcaatgccaatgtgggctcattatgcaaataatcataaaggattctgtgtttcttacgatttaagtgaaaatataaacttagaactacgctctgtaacgtttcctgtgcaatatacgaacaagagaattgatattactagtattatggaacatacagttataaatattttaaatagaatatcttcatcaacagtaaaccaaattatgcttgatgatatgttaatgatctatgctccaatatttctatcaaatataaaacatgatttttggaattatgagaacgaattcagaattacagtaggtaatgataaaaacatgaaatatataaatgcaattcctaaagcattttttttaggtattaaatgcgaaaaaaaaaatcgcgagagattgcaagaaatagctgtcgatcttaaaattccagtatatgaaatgttttatgatgatatggatgataattttaagttaagcaaaagacaaatttatccactattggactttaagttataa

SEQ ID NO: 13 - RNA, 543 nt, Lactococcus lactis
  source 1..543: mol_type = transcribed RNA
atggaattaacctttggtttaaaagaacgtttaatcttagctaatcagtatgaaattttatcaaaaatttcagaggacaaatctgatgctagctacttcgagaatttagcagagatttttaggtctggttatactagaaattacgaggaagcttttaaaggtttcgaagaaaatgagcttagccaagaagattgtaagtttgttgtagatgttttgaatttatatcgtgaattatataagacgtgggaagagttagaagaagttcatgagatcattgaagaagacgatgtattatttcagggatttgatggaaatgaagaagatcaatattatgcttattgcaaattcttagttgaagatcaagggttgtggggagagctaaaagatcttatgaaagaaggaaaaattaacttgaattcccaccggccaatgagagataaatatgaaaattatttattaaaacttaaagaaattcaaagccgaaaagaatatgatgtctcagaatatttgacacttgaagaaattgaatacattttgaagtaa

SEQ ID NO: 14 - RNA, 1179 nt, Lactococcus lactis
  source 1..1179: mol_type = transcribed RNA
atgaatggattatttggtgataatatattctttctatttctttttacttgtataacaataatgaactattccagaatgtctgaaactcaaaaaatgacattattttatttaatgacatttggtttagtcctattaaaaattataagcttcactgaaagtatattatatttatttattattttatttctctatcttgaatttttaacagaggattctaaaaaaaataaatatattacaaaacttagatacaagattctagacttcttgtttttgattttttttcagtatcaattcacactgttgttaatttcttttatctttgctagtacaatggttcaaactaatatttcaaaaattagtttatttccttttactctcagtaattccttctttcaattaatttcgataatattttttttcatagcaatatttaaaatgtctcaagaaaagtttaaagtaaagagtttatcttccttaattaaggatgtgtttcaaccctcgataaatgaaagtcatttagacccttacacaagatactttccaattttggcagacatggaagataaaacctatctttcaagaaaaaaaaacagccattgtttttcattcgaaatactgttatctaaaatgaagactttttatcggtcaaaaaagaatataggattctcagagacactcaaaatatataataacattccacaaattattcgtggttattcaactatagaaatgcagctagtaaggagcatttgtattgagtcaggatataataagctactctcaagaaagatatttgaaattctgtatacaaaaattatttttaatagtatgcaggattattataaaaaaaataactatgtaaataacgagcgattcagagagtacttattgataatctacatttcgaatgtcaggactaaaatcaatggaactatttactcaaatatgaccaaatatctgggagaggatgtttctaattggacaaaaaacaaattctacattgcttgcttaggattgccttatcaaacattcacgaaacattatatttttggtacgcatcaaaatatcattattaaatattcactggatactaagtttatagagacagagttggaaaggataggaaatattgagcaaaaatattacggcgcagataaggaccacaacaccctaatataa

SEQ ID NO: 15 - RNA, 1362 nt, Lactococcus lactis
  source 1..1362: mol_type = transcribed RNA
atgcaattaggagcaaaaatgaaattagaaaacctattcatagaaaaattacatggtatttataattatgatgttgattttaatgaagatattacttttttatacggaagtaatggatgtggaaaaacaacaatactaaatatagttaccagcattgtaactggaaaaatatatgaattatttttttatgactttaagattattaaattgaagtattccaacaacgataatgttgatgttattgaaataaaaagaaagcaagatgataaaaatagattaaaaataatttttgagggtagaaatgaaactatatcaaaattagataatttgaattttcaaagttcaagaagaaagatattattaaaggaatattttcatgaaaataatatattaaaggatatttctcgactatttaactatgtttatctacctttaagtagatcagcttatgttgatcctataaggtcatccagaaaaaacttttggatagaggaagaggaaatagaagaagagttatatgtaacaaaagatgcctcattaacacgagtacaagaactcattgttaataaaattttaactataaataatgaagttaaccggttaaataataactttagagataaaatcttgaaaacatcaattaatatttattcagaaaagaaaaattttagtgatatgataaatgaaattgagaattcaaatgaagaaaaaatactagaaatacaagaatcatacttaaatataatgacaaatttaaaattaattaatgaaagagaaatagaaaaatacaataatttcttttctagttacataaaaaaatatgaacaaataactgatgaaaatgatgggaaagtaaaacattttccaattaatttagtattacaatattcagagatatctaaaataagaaaagttgttgaactagctcaacagatggaaaaagataaaaagcaagcagaaaaacctttggaattattctgtagtacattaaatagttttttaagcagaccaggtgatattgatagtaaaaatataataattgaaactattggtcaacctggagtttattttaagataccgaataatcaaaacaaattaagtgtccaaaaattatcatcaggagaaaaacagttgattatattttttgctaatttgatcttcggagttaatgatagaaaaaatggtatttttatagttgatgagcctgaattatcactacatctaacttggcaaaaattatttatagagcaaacattaaaaattaataataatattcaattgatatttgcaacacattcaccagaaattgtaggtagacatagagataaaatgtataagttagaaaaagtgttaggagaatag

SEQ ID NO: 16 - RNA, 843 nt, Lactococcus lactis
  source 1..843: mol_type = transcribed RNA
atggaagagttagcatattcagatgaaggagttatttccagatattttttctttgaaggcgtaaataaactggatgtttttgttgaagatgtggggaaagaatacgagtatgaaactatattaaaaaaactatttggagagaaatataaaatatcggaaatctttcctttgggtgggaaaaacaatgttattaaaaccttggatgaatttattgaaaatggaaaaattgatagtgaaattcctaaaatattcattgtagatggtgattttgataaatatttagataatattgaagtcaaagaacatcctaatttggtttatcttgaaaaatataatatagaaaattattacgttgataaaaaagccacagtatcttttatgaaggggaaattagaaaaattagataaaaaagtagaagaaataattaaatttgaagaatggttagaagaaacagtaaatgatttaaaacctatttttatcctgtattgtatcattcaagaaaatgaattaggaatacctaatgtaggagaatcggagtataggatttttgaagatttatcaggtaaggtaatatctcaggcatatgattcattgaagttacaagttgaaaaagaattacccaatgtttcagagttagaacgaataatggaagaaaaatatctgaaagaaaattctgatttttttgatttaatttgtggaaagcatttattatgtagtttatttttacatttaaaatcctgtggaaagggattcagaagagctgatttagaatggcatttgaagcaaaatattgatatttcaaaactagattttttaaaacaaagagtagaaactttagtttag

SEQ ID NO: 17 - RNA, 573 nt, Lactococcus lactis
  source 1..573: mol_type = transcribed RNA
atggaaaataaaaaaaaatatctttttaaagaaatacagacttttttagtgaatgttggttggacacataaaatacaaatttgccaggttgatgtatatatggagctttcaaaaaatttaaaactggctaaaattattttgacatctttaacttctgctggtttggggagttttattttgaagattttcccaagtattcaggacatagccatgactattacatttgcaatatcgcttgctacaacaattgttatagctttagataaggaaaaagattataagaaattagctgaacagaataaagtggctgcagataaatacttagaaatgcgtaataaagctatgtcgcttctttatagaatgaaatatgaagaaggtattactacaattgaaaatgaatttttagaattgaaagaacttagaaatgaggcagatccaggtttaccacatacatcttatcaagctgttgaactagcaagtaaaaaattgaaagaaaataaagataatgattacactgaggattacaaattatttattcctaaagatttattagacttagaaggtgagatatga

SEQ ID NO: 18 - RNA, 1227 nt, Lactococcus lactis
  source 1..1227: mol_type = transcribed RNA
atgattattgactatgcaaaaactttagaacgttcagaagaggaaattataaaatgggaaaatagaattattaagattaataataagctatgtgataaatattacctagaaagagacaattactctatacttgttggatcagttggaagaggaacagcaattacgaaaactagtgattatgacgtgctatttaaacttcctcagtcgctttacacaaaatttgataaatatgaatcaaacggtcaatctcagttacttcaagaaattaaaaatataatcaaagaattacatcctaaaacaaaaatgaagggagatggacaagtagtttcaattgaatataatgatggggtaattgaacttgtcccagcatttgaacagagtgatggtaatttcaaatatcctgatagtaacaatgggggatgttggaaggtaacaaaaccacttccagagattgaaggctcagtaaaaaaatctaaaaatacttctaaaacttttaattacttgtgctttctgatgcgacaatggaaaaatcataatggcttttcttttaaaggattgcttattgacacattagtgtctgagtatcttgaagatgaaagttacaaaaataaaacagagttacaattattagaaggtttttttggtcaattatctaaagaaaataaagagcaatcatattggtatgccttagggagtaatcaaaaaatctataatgacgataatggtaaatttgtaaccaaagctaaaaaagcattaaaaaaatttgatgaggctacggaagaatcaattttaaaagatttgtttggctataaacaatcagaacaacaggctaccaatgaagaattcattgagaataaatttattgtagatattcgatttaaccttaagattgattgtgatgttatacaaaatggttttagaggtatgaaacttactgaatatatacgaagtaaatttagactaggacgtggaaaaacattgaatttttctgtgattaatagcaatattccctcttatctaccagtaaattattattggaaagttagaaatgttggtagagaggctttaggtaaagagagaggccaaattttcttgggttcgaaaaaactaatcgagcatacaagctttaatgggaaccattatgttgaatgctatgcaatatctgatgggattgtgattgctcgggataagattaaagtacctattgatgtattgcatgggagatag

SEQ ID NO: 19 - RNA, 2205 nt, Lactococcus lactis
  source 1..2205: mol_type = transcribed RNA
atgcaattaggagcaaaaatgaaattagaaaacctattcatagaaaaattacatggtatttataattatgatgttgattttaatgaagatattacttttttatacggaagtaatggatgtggaaaaacaacaatactaaatatagttaccagcattgtaactggaaaaatatatgaattatttttttatgactttaagattattaaattgaagtattccaacaacgataatgttgatgttattgaaataaaaagaaagcaagatgataaaaatagattaaaaataatttttgagggtagaaatgaaactatatcaaaattagataatttgaattttcaaagttcaagaagaaagatattattaaaggaatattttcatgaaaataatatattaaaggatatttctcgactatttaactatgtttatctacctttaagtagatcagcttatgttgatcctataaggtcatccagaaaaaacttttggatagaggaagaggaaatagaagaagagttatatgtaacaaaagatgcctcattaacacgagtacaagaactcattgttaataaaattttaactataaataatgaagttaaccggttaaataataactttagagataaaatcttgaaaacatcaattaatatttattcagaaaagaaaaattttagtgatatgataaatgaaattgagaattcaaatgaagaaaaaatactagaaatacaagaatcatacttaaatataatgacaaatttaaaattaattaatgaaagagaaatagaaaaatacaataatttcttttctagttacataaaaaaatatgaacaaataactgatgaaaatgatgggaaagtaaaacattttccaattaatttagtattacaatattcagagatatctaaaataagaaaagttgttgaactagctcaacagatggaaaaagataaaaagcaagcagaaaaacctttggaattattctgtagtacattaaatagttttttaagcagaccaggtgatattgatagtaaaaatataataattgaaactattggtcaacctggagtttattttaagataccgaataatcaaaacaaattaagtgtccaaaaattatcatcaggagaaaaacagttgattatattttttgctaatttgatcttcggagttaatgatagaaaaaatggtatttttatagttgatgagcctgaattatcactacatctaacttggcaaaaattatttatagagcaaacattaaaaattaataataatattcaattgatatttgcaacacattcaccagaaattgtaggtagacatagagataaaatgtataagttagaaaaagtgttaggagaatagatggaagagttagcatattcagatgaaggagttatttccagatattttttctttgaaggcgtaaataaactggatgtttttgttgaagatgtggggaaagaatacgagtatgaaactatattaaaaaaactatttggagagaaatataaaatatcggaaatctttcctttgggtgggaaaaacaatgttattaaaaccttggatgaatttattgaaaatggaaaaattgatagtgaaattcctaaaatattcattgtagatggtgattttgataaatatttagataatattgaagtcaaagaacatcctaatttggtttatcttgaaaaatataatatagaaaattattacgttgataaaaaagccacagtatcttttatgaaggggaaattagaaaaattagataaaaaagtagaagaaataattaaatttgaagaatggttagaagaaacagtaaatgatttaaaacctatttttatcctgtattgtatcattcaagaaaatgaattaggaatacctaatgtaggagaatcggagtataggatttttgaagatttatcaggtaaggtaatatctcaggcatatgattcattgaagttacaagttgaaaaagaattacccaatgtttcagagttagaacgaataatggaagaaaaatatctgaaagaaaattctgatttttttgatttaatttgtggaaagcatttattatgtagtttatttttacatttaaaatcctgtggaaagggattcagaagagctgatttagaatggcatttgaagcaaaatattgatatttcaaaactagattttttaaaacaaagagtagaaactttagtttag

SEQ ID NO: 20 - RNA, 1800 nt, Lactococcus lactis
  source 1..1800: mol_type = transcribed RNA
atggaaaataaaaaaaaatatctttttaaagaaatacagacttttttagtgaatgttggttggacacataaaatacaaatttgccaggttgatgtatatatggagctttcaaaaaatttaaaactggctaaaattattttgacatctttaacttctgctggtttggggagttttattttgaagattttcccaagtattcaggacatagccatgactattacatttgcaatatcgcttgctacaacaattgttatagctttagataaggaaaaagattataagaaattagctgaacagaataaagtggctgcagataaatacttagaaatgcgtaataaagctatgtcgcttctttatagaatgaaatatgaagaaggtattactacaattgaaaatgaatttttagaattgaaagaacttagaaatgaggcagatccaggtttaccacatacatcttatcaagctgttgaactagcaagtaaaaaattgaaagaaaataaagataatgattacactgaggattacaaattatttattcctaaagatttattagacttagaaggtgagatatgaatgattattgactatgcaaaaactttagaacgttcagaagaggaaattataaaatgggaaaatagaattattaagattaataataagctatgtgataaatattacctagaaagagacaattactctatacttgttggatcagttggaagaggaacagcaattacgaaaactagtgattatgacgtgctatttaaacttcctcagtcgctttacacaaaatttgataaatatgaatcaaacggtcaatctcagttacttcaagaaattaaaaatataatcaaagaattacatcctaaaacaaaaatgaagggagatggacaagtagtttcaattgaatataatgatggggtaattgaacttgtcccagcatttgaacagagtgatggtaatttcaaatatcctgatagtaacaatgggggatgttggaaggtaacaaaaccacttccagagattgaaggctcagtaaaaaaatctaaaaatacttctaaaacttttaattacttgtgctttctgatgcgacaatggaaaaatcataatggcttttcttttaaaggattgcttattgacacattagtgtctgagtatcttgaagatgaaagttacaaaaataaaacagagttacaattattagaaggtttttttggtcaattatctaaagaaaataaagagcaatcatattggtatgccttagggagtaatcaaaaaatctataatgacgataatggtaaatttgtaaccaaagctaaaaaagcattaaaaaaatttgatgaggctacggaagaatcaattttaaaagatttgtttggctataaacaatcagaacaacaggctaccaatgaagaattcattgagaataaatttattgtagatattcgatttaaccttaagattgattgtgatgttatacaaaatggttttagaggtatgaaacttactgaatatatacgaagtaaatttagactaggacgtggaaaaacattgaatttttctgtgattaatagcaatattccctcttatctaccagtaaattattattggaaagttagaaatgttggtagagaggctttaggtaaagagagaggccaaattttcttgggttcgaaaaaactaatcgagcatacaagctttaatgggaaccattatgttgaatgctatgcaatatctgatgggattgtgattgctcgggataagattaaagtacctattgatgtattgcatgggagatag

SEQ ID NO: 21 - protein, 303 aa, Lactococcus lactis
MEILKKRINNVEKYLSDISDNQNFFIGIKIEPNKIQKILSEKFNKTDENGLMFIPKPYNGIMAQRNTVGEFLSDKSKPKETAYRAQSWSLKDWGGNIHSGTSEVPYKRYPKIFIDPFGLNFILLNDKQYLVINKQFNKKKSEYDKIKSAINLTLELFNKAETFILDVDTDMLKPVRKSVPWEILPQGVRIWNAFKDVANSNQVSKSAKILIEERFKYLEEFKPDVEYQGTAGYSGYIAFAFSSKNIYVLDSILYGNATYIFGGNWEEISQLTKKQIIDDNLYKYRIIHDKNWKIEIGNILNSI

SEQ ID NO: 22 - protein, 316 aa, Lactococcus lactis
MKFEDYKLEIPDRDPVSEENFDIETWREENPIDYLKALHLIAKQHSIQKCAFDTIFTVLKLYLPNILYKYYSLNENMDLNNQKFETIKNQKIYTAETKALNDPFEGKAFYYDPNKLMKFKPLDKVDGHLIDDFSDYVRIASLTGNGTDSMPMWAHYANNHKGFCVSYDLSENINLELRSVTFPVQYTNKRIDITSIMEHTVINILNRISSSTVNQIMLDDMLMIYAPIFLSNIKHDFWNYENEFRITVGNDKNMKYINAIPKAFFLGIKCEKKNRERLQEIAVDLKIPVYEMFYDDMDDNFKLSKRQIYPLLDFKL

SEQ ID NO: 23 - protein, 180 aa, Lactococcus lactis
MELTFGLKERLILANQYEILSKISEDKSDASYFENLAEIFRSGYTRNYEEAFKGFEENELSQEDCKFVVDVLNLYRELYKTWEELEEVHEIIEEDDVLFQGFDGNEEDQYYAYCKFLVEDQGLWGELKDLMKEGKINLNSHRPMRDKYENYLLKLKEIQSRKEYDVSEYLTLEEIEYILK

SEQ ID NO: 24 - protein, 392 aa, Lactococcus lactis
MNGLFGDNIFFLFLFTCITIMNYSRMSETQKMTLFYLMTFGLVLLKIISFTESILYLFIILFLYLEFLTEDSKKNKYITKLRYKILDFLFLIFFQYQFTLLLISFIFASTMVQTNISKISLFPFTLSNSFFQLISIIFFFIAIFKMSQEKFKVKSLSSLIKDVFQPSINESHLDPYTRYFPILADMEDKTYLSRKKNSHCFSFEILLSKMKTFYRSKKNIGFSETLKIYNNIPQIIRGYSTIEMQLVRSICIESGYNKLLSRKIFEILYTKIIFNSMQDYYKKNNYVNNERFREYLLIIYISNVRTKINGTIYSNMTKYLGEDVSNWTKNKFYIACLGLPYQTFTKHYIFGTHQNIIIKYSLDTKFIETELERIGNIEQKYYGADKDHNTLI

SEQ ID NO: 25 - protein, 453 aa, Lactococcus lactis
MQLGAKMKLENLFIEKLHGIYNYDVDFNEDITFLYGSNGCGKTTILNIVTSIVTGKIYELFFYDFKIIKLKYSNNDNVDVIEIKRKQDDKNRLKIIFEGRNETISKLDNLNFQSSRRKILLKEYFHENNILKDISRLFNYVYLPLSRSAYVDPIRSSRKNFWIEEEEIEEELYVTKDASLTRVQELIVNKILTINNEVNRLNNNFRDKILKTSINIYSEKKNFSDMINEIENSNEEKILEIQESYLNIMTNLKLINEREIEKYNNFFSSYIKKYEQITDENDGKVKHFPINLVLQYSEISKIRKVVELAQQMEKDKKQAEKPLELFCSTLNSFLSRPGDIDSKNIIIETIGQPGVYFKIPNNQNKLSVQKLSSGEKQLIIFFANLIFGVNDRKNGIFIVDEPELSLHLTWQKLFIEQTLKINNNIQLIFATHSPEIVGRHRDKMYKLEKVLGE

SEQ ID NO: 26 - protein, 280 aa, Lactococcus lactis
MEELAYSDEGVISRYFFFEGVNKLDVFVEDVGKEYEYETILKKLFGEKYKISEIFPLGGKNNVIKTLDEFIENGKIDSEIPKIFIVDGDFDKYLDNIEVKEHPNLVYLEKYNIENYYVDKKATVSFMKGKLEKLDKKVEEIIKFEEWLEETVNDLKPIFILYCIIQENELGIPNVGESEYRIFEDLSGKVISQAYDSLKLQVEKELPNVSELERIMEEKYLKENSDFFDLICGKHLLCSLFLHLKSCGKGFRRADLEWHLKQNIDISKLDFLKQRVETLV

SEQ ID NO: 27 - protein, 190 aa, Lactococcus lactis
MENKKKYLFKEIQTFLVNVGWTHKIQICQVDVYMELSKNLKLAKIILTSLTSAGLGSFILKIFPSIQDIAMTITFAISLATTIVIALDKEKDYKKLAEQNKVAADKYLEMRNKAMSLLYRMKYEEGITTIENEFLELKELRNEADPGLPHTSYQAVELASKKLKENKDNDYTEDYKLFIPKDLLDLEGEI

SEQ ID NO: 28 - protein, 408 aa, Lactococcus lactis
MIIDYAKTLERSEEEIIKWENRIIKINNKLCDKYYLERDNYSILVGSVGRGTAITKTSDYDVLFKLPQSLYTKFDKYESNGQSQLLQEIKNIIKELHPKTKMKGDGQVVSIEYNDGVIELVPAFEQSDGNFKYPDSNNGGCWKVTKPLPEIEGSVKKSKNTSKTFNYLCFLMRQWKNHNGFSFKGLLIDTLVSEYLEDESYKNKTELQLLEGFFGQLSKENKEQSYWYALGSNQKIYNDDNGKFVTKAKKALKKFDEATEESILKDLFGYKQSEQQATNEEFIENKFIVDIRFNLKIDCDVIQNGFRGMKLTEYIRSKFRLGRGKTLNFSVINSNIPSYLPVNYYWKVRNVGREALGKERGQIFLGSKKLIEHTSFNGNHYVECYAISDGIVIARDKIKVPIDVLHGR

SEQ ID NO: 29 - DNA, 22 bp, synthetic construct
  source 1..22: mol_type = other DNA
ctaatgtcactaacctgccccg

SEQ ID NO: 30 - DNA, 18 bp, synthetic construct
  source 1..18: mol_type = other DNA
gctttatcaactgctgct

SEQ ID NO: 31 - DNA, 21 bp, synthetic construct
  source 1..21: mol_type = other DNA
tgatttcgttcgaaggaacta

SEQ ID NO: 32 - DNA, 19 bp, synthetic construct
  source 1..19: mol_type = other DNA
tggcggacaataagtcctc

SEQ ID NO: 33 - DNA, 46 bp, synthetic construct
  source 1..46: mol_type = other DNA
agcagcctgcagaggaggcactcaccatggaattaacctttggttt

SEQ ID NO: 34 - DNA, 32 bp, synthetic construct
  source 1..32: mol_type = other DNA
agcagcggtaccttacttcaaaatgtattcaa

SEQ ID NO: 35 - DNA, 21 bp, synthetic construct
  source 1..21: mol_type = other DNA
gtcatcaaacatactttcgtc

SEQ ID NO: 36 - DNA, 21 bp, synthetic construct
  source 1..21: mol_type = other DNA
aagttttgccattgtttctcc

SEQ ID NO: 37 - DNA, 21 bp, synthetic construct
  source 1..21: mol_type = other DNA
gtcatcaaacatactttcgtc

SEQ ID NO: 38 - DNA, 21 bp, synthetic construct
  source 1..21: mol_type = other DNA
aagttttgccattgtttctcc

SEQ ID NO: 39 - DNA, 19 bp, synthetic construct
  source 1..19: mol_type = other DNA
acgcctattggaaccactc

SEQ ID NO: 40 - DNA, 20 bp, synthetic construct
  source 1..20: mol_type = other DNA
ttttgtcttgtccccttctc

SEQ ID NO: 41 - DNA, 31 bp, synthetic construct
  source 1..31: mol_type = other DNA
atggaaatattaaaaaaaagaattaataatg

SEQ ID NO: 42 - DNA, 22 bp, synthetic construct
  source 1..22: mol_type = other DNA
agatagaattaagaatatttcc

SEQ ID NO: 43 - DNA, 25 bp, synthetic construct
  source 1..25: mol_type = other DNA
atgaaatttgaagattataaattag

SEQ ID NO: 44 - DNA, 24 bp, synthetic construct
  source 1..24: mol_type = other DNA
ttataacttaaagtccaatagtgg

SEQ ID NO: 45 - DNA, 21 bp, synthetic construct
  source 1..21: mol_type = other DNA
atgcaattaggagcaaaaatg

SEQ ID NO: 46 - DNA, 25 bp, synthetic construct
  source 1..25: mol_type = other DNA
ctattctcctaacactttttctaac

SEQ ID NO: 47 - DNA, 22 bp, synthetic construct
  source 1..22: mol_type = other DNA
atggaagagttagcatattcag

SEQ ID NO: 48 - DNA, 22 bp, synthetic construct
  source 1..22: mol_type = other DNA
ctaaactaaagtttctactttg

SEQ ID NO: 49 - DNA, 31 bp, synthetic construct
  source 1..31: mol_type = other DNA
atggaaaataaaaaaaaatatctttttaaag

SEQ ID NO: 50 - DNA, 21 bp, synthetic construct
  source 1..21: mol_type = other DNA
tcatatctcaccttctaagtc

SEQ ID NO: 51 - DNA, 23 bp, synthetic construct
  source 1..23: mol_type = other DNA
atgattattgactatgcaaaaac

SEQ ID NO: 52 - DNA, 21 bp, synthetic construct
  source 1..21: mol_type = other DNA
ctatctcccatgcaatacatc

SEQ ID NO: 53 - DNA, 30 bp, synthetic construct
  source 1..30: mol_type = other DNA
atgaatggattatttggtgataatatattc

SEQ ID NO: 54 - DNA, 22 bp, synthetic construct
  source 1..22: mol_type = other DNA
ttatattagggtgttgtggtcc

SEQ ID NO: 55 - DNA, 126 bp, Lactococcus lactis
  source 1..126: mol_type = other DNA
atgagaaagcttacaagtattggagccattgctttacgtggaatatcactgagtggttattctcaggataaagacacagctgaaacaataagtactacacagagcacagtaagaaatttaatataa

SEQ ID NO: 56 - DNA, 768 bp, Lactococcus lactis
  source 1..768: mol_type = other DNA
atggaaacgataattaaaaataaaaaaatagccataattattgcaataatagttgtcttaggcactatactgttaggagcataccatcaatcagtaaacagatattcttctaaaattgctgtacagattgataatgttgaaaaattgtctaaaaacaattttgcaccacaaaatcatgttaatgattacggtcaatggattgtattggctaaaaatgcgaataacaagtcaaatttatttcaaatgataaaaatgactagggaatatgacaaaaaattctctaaggatgaatctgaggctaagaaaaacgaaaaaaaattaacagacgaagtaaataataaatggataaaaatatatacacaatccatgcttaaagcatcagaatcagagtcttatggtaataaaatcactaaagtctggagagatgctatttttgaaaattatgccactgtaaatggaaaacactattatgattttaaagatgctatccatgcttttcagacacagtatagtaatgagtttagtgaattgaatagtttctttcaagacaatattgactctgcaaacaacctttcagaattaataagaaaatattttccagaaaaatcaaatagagctgatgatttagagaagttcgctcaggatgtttttaatttcaacgatgaagctataaatccatctggttcatattcaactcgttcatctaaattgagcgagttagatactaaggctttatctagttctaaaactgttggaatgaggttctaa

SEQ ID NO: 57 - DNA, 1284 bp, Lactococcus lactis
  source 1..1284: mol_type = other DNA
gtggctggaaaaaagaataatgcaacacctagttggagtggttataatcatcaaggacaagttggtatatttttagctttaaatgaattaaataagttaatccaagataacatttcgcatgaagattattctgtacaatttgaaaaagaagatggtgaagatatagatatcgttcatagcaacaaagtcatttcaagacaccaagttaaggcgaaaacaacttcaaaaaatctaaatgactacagtgatgtgcttacggggtttaatattgatggagtggataaaaactttagatatttacatacaatttgtgaagtaattggatttggtctaccaaaagaggagtttgaaaaactaaaaaataaacctaaatatattccaaatagtctaaatattaagttatataagtatcctgatggtatcatgtattgtgaattaaattgtaataaagaatctaaaatagacactttttgcaagaaagaaataataaatatactcaaaaaactcaattctagtctcaaagatgatgatgagcatattgaagaaactttatttgggttaaaagatctactctgtagcaaaataagaaaggcccatgaaaaaggaaaagaagaacatccaatcatttcatttcaagagatttattattttgttacttctacccaaaaacgagaaagacaatcaattcatagagcaaaaactctctttaaacttttttggaatgaaagtcttaatgatgttgttgatgatactgatgatgttgatgatacaattattattaaaattttaaacctggcagatgatgattttcagaggctattaattgatttgcatcccgatcgcaatatttcacagctacaaaaggggaataatatagataatttaattgatgaaaataactttaaatatattttgtatagttttattatatggtgtaaaaaagaaaattttatattagataacttatactatcaaactaatcaggattcgtttaggctaagtttgattaacgtaccaaaacctgctgcaggtgacgttaaaaagaggattatgaaaaatataaaatttataagagctagttttgatacaaaatatttaatcaatatgcatattgatggaacaaggttttttgaagataaaccaacacatgaagaagggaaagaaaaattaaagtctgggttagttggaaagagggcagatagtatattttcgtctaatttggaatatattgattgtgtgaatactatcgaaaaattgagggaggatcaacataatgatgtataa

SEQ ID NO: 58 - DNA, 729 bp, Lactococcus lactis
  source 1..729: mol_type = other DNA
atgatgtataactttataaaacaaatttttgaaaaaaatgattatcaccaatatgttaatatcattgagttgtggaagaacagcaatgaattttttatacttcaagaatatgataaggaagagcttcaatcaagtagtaaaattgacgaaaatttttttacttgtgtacagacaaataacatagtttctgaatttgaaaaaattaaagataataaaatcaagaaaaatacatctttatttataattgtgaaagttaaaaaccttgaaaaagaatataaaaggctcaagaatataacgatgagaattgaggaagatgagtattattttagaaaatatgttattttctatactgaagatggccttagtaaattaaagcccgatgctcactctctacttgattatatccaaactgaagtccctaaaactacatcatctgtaggaaacaatttatttgatacattcgaggagaatatgttctttgatgatgcttactttattgctatgcaattaattattaagttaacatttatttcacttcctcattctgacaaatattttgaaacggttgaagataaaatcaaaacgtgcattgaattgaaaggtttaattgataaagaaaatcaggtaaatcaagttttaaaactatttagtgataataattttaaagagaagcttgaagatgaaactagcggtttattggagaatttaaataaaattttaggagaatga

SEQ ID NO: 59 - DNA, 2652 bp, Lactococcus lactis
  source 1..2652: mol_type = other DNA
gtgaagattaagaagattttactttataatttcaagaattttaggaataaaacagttatcgattttagtgatggaataactttcttagtagggccaaatggatttggtaaaacgacaatttttgatgcaattgaattaggattaacaggtagaatttctcgtacaaataaagtaacgggagaaaacatttcgtataataaacctttctttcaaaatgaaattggtcatcccgtagtcataaagttgtggttagaaaaaattaatggagagaaattagttattgtcagaagattaatgaatagttctactgatggtaaaaaagcatttgctcctttgaaatctacaaatcaatttaacctgtttagccaagaacaaatcaataattcaagttttaaaagtatagataacattgatacattacaacctattaaccaaagttcaatagatagctttttagggattgagggaaaatgtaagatagaaaacattttcaatttattcaactatatccaacaagaagagacaacattttttctaaagcaaactgaacaaagaagaagtgatcttctatcatttttggtcaaaactgatgaagtagaagaaaaaatagggaaaattgacaaaataactagggtaattaagggtaaactagatattctgacagaaaaaaagaaaaaattcactaagcgtgagctagctgatattccttatcagagactatttagtcatagagaatttgcatttgatatttcaaatcctttttcttatgaaaatattgaccaactgccaatttataaaaacaatatacagaatatcattaattttaagcagaatttttcgattccagaatataaaaggaagattgaagttgaacataaaatgaaagaaatagatatcgatcgagatattgaacaggctttctattactcaattcttgcaccaattattcgtaggcccaattatcactggcaatgggaaaaatatatagtagaaaatccaatattatttgagtacgtacttttagaaaattatcttcaaaaatatgatactatcgttcgaaatcatgaacgaagaaaacaactcaacgattacagaaataaattatctgaagagctttctgatatgacagaaaaatctttgaattatgttcagaatgatagtctagcaaatgattttgaattacttaaatctcaatttatacaatatcggacattaagaagtcaagctagtcaaattgataaaaatttaagcgatttaaagcaactaagaaataatttagacaaacaatttgatgaattacggaaacatcaacatgttgatgataataggtgcccattctgtaattatccatatggttctttcgatgaattaaaacttgcttatgataattataaagattatctgacaagtatttcatctcaaaatagccaagatttacaagaagcacaagtatttattgattcaattattacacaaataaaacaaaaaatatctaatgaactagatgaattaggtgttgaagtagacaaacaaatattagagaaattacaagagttacgcaatcaaaaatcaaagtattctgtaaatattgatagttttaaaaacttcattcgatcctatacaactattttaccctatgagctggttaatttatcatttactatctataatcaacagtataagttaagacttcaagaatttgattctaagttagagattgaaagtgatatatatactttgctagatattaaaaaattagaaaatacggaacagaaatttgaagagttgagaagcgagtattctggattaaattttgaaacatttcaattggaaccaacttcaaatcataaaattagtaaatctatggttgatagtcagttgtataaattaagagaacaacttaaatcagaagttaacaaggtgtattccataaaccataacttgataagtgacccaaatataatactaataaattattttaacaaagaccttgaacacttagaaaatatcagcattcaagatttggaacttaaaaaatcttatcttgataagcagagtaacttagttcaaaatcagcaaatccgaattttatctagtgagataagaatacttaaccaaacatgcgaacagttagaagaaataaacaatgtttacaaagctgaagttaaacagtttaaaatagatattgttaaacagttacgtatcccattctttatctattcagcaaaaatgttacaaaattatcaacaaggtctaggaatttttttaacatacaagaaagcaacagaaaattcagagaataaggcaattattagatttaaatccgatcctgagaacgaccatgatgccatgaaccaattaagtactggtcagttggcagttgtctcacttgcttttactcttgctttaaatacaatgttcaagctatccgataatttaaatttcttgatgatcgatgatccgatacaggatatggatgcaatgaatgtattatcatttattgagattttacgtcacggaatcatagataagtatcaaattattttatccacttatagtgaccaaaatgctctattcatggggtataagtttgtaaatgccaattttctagtggatattgattataagaatataagggaattacaaggctaa

SEQ ID NO: 60 - DNA, 1080 bp, Lactococcus lactis
  source 1..1080: mol_type = other DNA
atgaaaaagtatactattggtagaaatgagaaatgtttttgtggtagtgatattaaattcaaaagttgctgtatgaggaaaaaagatttattggaaagagagagcatagatgcggagagctttaataaattatacagcaaggggaggaagcaggctcgttttagagagtgccaccaccctgtaaaaaacgagtgttctgaaaaaataatttctgcacattcaatacaaaacaataagatactatcaaaaatagctgatgatggtgttgtactaatgcctattccaaaaaatttaaattttaatgaacaaacagaatacggaagaaaggaggctactacttttacaggtttttgtaatgagcatgataagtatacttttagacctattgaagataaagactttgagggaagtgaggaacaagtatttctacatatttatcgagcatttgtgctggaacttcatagaaaggaagaagggcatagattttatcaatatttattttcgatgatgccatcaattgcaagaaattcggaaacaatgaagcaattgtgggtttccaaaataggacacgacgacttgttgatagaaaaggaagagtttgacaaagcgataatagataataaatacaattgtttaacctattttgtctgggaatttgaaggagaaagtaacttcgctgcaactggtatggaagaaccaaggtttgattattctggactaagaattcaagatttagaggactttgatattccggctaaacacatttactatacaatttttcctgataatggaaaaatgtatttcataattgcttggctcaaaagtaatgatgaactattttgtaccattggtgagaaattaggcagcattaatgaggaagaaaagaaaaattttgttaataatgttgtaccgagtacaacagaaaatctagttattaacccaagctcatggggagcgatgaaaagaaaagaaaaagaaaatttcaattccattttccaggggaaagaagtaatgttggaacttctaggaaaattagttgatagattctcttatcctggatatgatttatttaaattgtaa

SEQ ID NO: 61 - DNA, 639 bp, Lactococcus lactis
  source 1..639: mol_type = other DNA
atggagtacgatttagagtttcttaaaaatttcgatgggaatattctcccctctcaaattaatatatcagacctggtaaacgttactgagaagcatgtttctcaatttcgttacttttacagtagcgataaatctgaattcccagaaataattattgaatttaaaaacctgcatatccctcatttactcggtttgtcaaaagatcaccatcttaatctttctacctatcaagcaagagaaataattaataatttaaaacgtgactggaatttagagtacttaaaaagtagtgatgaacaatggtttgctgaaaataaagacaagttagtaggggtgcttcttctctatcagctactacatgttcaaaactgtagagtgttaacacctttatacattataaattcaaattttggtcgaagatttaaacgagacaatgtatattttgtaattcttagatcaactaataataaagaatacacgatagaactagctccaatgaaaaatcatgacaatgtatttattccaaaaagtttgaagattaatgatactcatgtgcataaatgtagtgaaatcagtctaactttcttgagatctgaaagaataaaatatgataaaaaaagaggaaagggcagaaaataa

SEQ ID NO: 62 - DNA, 861 bp, Lactococcus lactis
  source 1..861: mol_type = other DNA
atgaggaaagaaaaaacttttaaaaaattttatttttggtcaataaaattttggattccaatattagtaattgtgtttgttcctatacttgtaagttgtttatcaagttctgggttgacttttaaagaagttaatggtgtagcgaatacaaattattatacaagagctatttatcaattagttcctaatcgaaagtttagtacatatataattatatggtttttaacactagtgataacgctaactagcatacgtaaaatgaagtcttcagatacattcaacggtaataattctatttacttttctgattgctattgttatttatggatagcagcaaatatactaggttataaaaaattccaattgattagtgtgtctcttcctttacaatttgaactcgtaattaatgaagtcttctctgagtttattgttgaaacgtctctcactccatatgaaaagtttgatggagatatatctataaaatataaaaatagagaatatacagcagatataatgaatgtattgatttgtgattcatataatatcagggagaatgatatagctgcagattttaatagttatccagtattaaagattagttcaagcaaaatggatgatgggataaggtatgataatcctaaattagtaagaagagtaagagaggagatgaataaggtttcgaaacaataccgagaactcaatatatttttaactactaatcccaaaaatactttgaaaattataggttcatccttcatggcttcggatagatttggatttgaaaaaattagtgtaatctcaatggataatgatagaatttataaaaaggcaaaagtaatatatcaaaacaataaaaattaa

SEQ ID NO: 63 - DNA, 1152 bp, Lactococcus lactis
  source 1..1152: mol_type = other DNA
atggaaaaaaaggaattattaaacaaaatatattgtgaattagctgatagtttgaatatatcagacacaatgacacaggaaataattagctcatataagtcggtagggagttatcttggaaacttagaagaaaatttaaatatacatatctatcctcaaggttctatggctttgggtactttagtaagaccgattgctgaggataaggagggggactatgatgttgatctagtttgtttactaaaaaatggggggaacttacccgctaaagatataaagcaaattattggtaagcgattatcagaaggggaaagatacaagtcgaaattggatgctgaaggaaaacgatgttggactcttaaatattctgattttcatatggatattcttccttcagttccaaaaatttctaccttacctaatccttcccgaaacgttactcaaattagattaacacataaggaaaatgattcttattttaatcgatatagtgatcctaaagcttatagaaaatggtttacaaaacaaatggaagtaagtttctctaaacagagggagattattgcaagaaataagaaagtcgaaattgaagaagtaaaattattcgatgtacgaactcctctccagatgactattcaaatattaaaacgacatcgagatataatgttctctggtagagataataagccaatttctatcatcattacaacacttgctgctaaatcatattctggagaaacaaatctatatgaagctatggaaaaagtgctgcaagatatggataaacatatagaatttaataatggcaaggcagttatatgcaatccgaccatggattcagaaaattttgcagataaatgggaggagtttccagaaaagcagcaagctttttttgagtggcttaataaagcaagaaaagatattatacaagaaccattagaatttgctggagggatgggaacaatgaaaaatcggatgcgagaactttttggttcgaaagcagtgaatcagacttttgatacatatgaagaaaaaaatattaaagagaagaataagggtaatctaggagtcacaaaagaagggaacattggtagtattgcttctagtaatataatagctccattaagcacacacacgttttatggtaagtaa

SEQ ID NO: 64 - DNA, 468 bp, Lactococcus lactis
  source 1..468: mol_type = other DNA
atggtaagtaaaaaatattatcccagaaaaaaaatattgactccaattcagcaattttctttagtcaagcaaaacttttcaaaggcaaagataataaacaaccagggtaaactacacttatatatatcgctccaaccatcagttttttctcccagctatcctattgaaattatcgaatactcacgtggtaaatatgatacttggctaattggaaatatacaaaaaattgataaccctaagtttcctcataagtatgaagtagacaaaaaaaataacaaagtgaggctatgcttgtatcatccaaataggggggagtggaccaaagatcaagagatttataatactctaattccttggacatgtgagtggttatattactatgagctttggttagatgatggaaaatggagaggcggaggagagcatccaaggctgaatgatagaaaaaggcctagaagaaataattga

SEQ ID NO: 65 - DNA, 1614 bp, Lactococcus lactis
  source 1..1614: mol_type = other DNA
atgagaattgatgattttctgtctaagatttcatttgaaacgtctacgtgggatgattggggttttagacaaactgccaatataacattcaaggaagaaagtggtgacaaaaattttttagtgaagatttgcccaaatactaatgaaaattttgagagaatcaaagcaaatcaggttgccaacgaagaaaaaaattttttttggggtcaaacgcaagagtattatgaattcatcttagattatttaaatgatgaagatgaccgaaacaggtggtttaagttgactggagatattgcgaaccaaggtcaaggttttgtaaaatattatgaaacaatgaaatttaaatatgatccagaattatttgaaagtaaagagaatgatagatttatagcagaattaaagatgttcgaagattcattttttagagatcaaacatttaatgagtggacagatttattaaagtctttacatcgattaacggttaataaatcatttctttctgattataaatttactgtaaaaaagaactctcaagatatattaaatgttgatgtcaagcctggtgataatatttctataaagttagataacgtttcattaccattatctttttcaaataatgtattttgtatcattggtgataacggaagtgggaagacaagctttataagagaattgtcaaaatccattttggatatttcttctgaattagtaatagaaaatattgaatttgacactcaggaagatgctaatattattaataaaattttatattgttcatttagcccttttgacgaaaaaattaatattgctcatgaagcagactcaaatgatgaaaaaaaatttgaatatatagggcttttaaattatgatgtagggaataaaactattggagaagaaattaccaaagacattttggaaagtttgatgtccgttaaaaacaatcaaaacaacaagtatctgctttggttagatgctatggaacgattatcttttgaaaaatggacttatgaaatagtttctatttttgaaaatgatttattcagatctgattcagagaagacagaagaaggggtgacgacaagcagatatatttataaaaattataatgaaactaaaaataaaattaaaaaattgagttctggacagaaaattttcttattgactattacaaagctagttttaaagttaacggaaaggacaattttatttattgatgaacccgagctatttttacatcctccaatggtcaaaacctatgtaaggataatatctgacttagtaagttatgtaaatggactaggttttatcataactcattctccaatcactcttcaagaatttcctaatgactgtataaaggtagcttatcgcaatcaattgggagaatatttaataaaaccaattaattttaaaacatttggagagaatataaatattattaatgataaggtgtttaatataggtcttcagcagtcagggtattataatttaattgaaagtcttaaatattctgaagacggtgaaaaaaaactaaaaaaaatacttaatttttcaggtagtgaagcaaaattactaatcaagttatttctagaaaaaaacgagaaagaataa

SEQ ID NO: 66 - DNA, 1332 bp, Lactococcus lactis
  source 1..1332: mol_type = other DNA
atgacgttaactataaaagatattttagaagtattgcgagatgaagtagtagaatttgttgacttcattggagacggtgaagatgaaagttttaatcaagttgtagatttattttcagcctatgaaaaaaaatacatggatgaatatgaatcactttccaatcttccagtaatttccgaggacgatattaatgcaaactttgctactataaaaaactattatccagaatactacaaaggtcatgctctatataaatatagggaaataatggaatcattagttgaaatgagatgtcctatttgtgattgttcctttgcatatagtcaagtaaccttagatcatataatacctaaaaaaaaatatccttactactctatcacacctataaatttagttccaacttgttataactgtaatatgcgaaagaatgatggtaccccatcaaaaatattacatccgtactttcatgattttataacttttgattatttaacagttactcttgaagtaaatgaaaaagaacccactgagtctattattaatcttggttttactgagttgaatagtgacgatgttaatgagataaacaaagttgagtcgataaaagctaacatagatttatataaacttagacaaaagtacactgatataattaatatctctttttctaaacttattgatgaattccaaaaagttgtaagtatacaaaatgatgtatatagtattaatgaactcaagaaatatttcaaattgatggatatttttatggatagtaacgattataattatgttgatgaggactttttgcgacatctatgtattgtagaaatcacccaaaacactcactttttaagttgcttagccgaaaagttaaacattttcgttgattatgatactcagatagtcgagtcaatggaaaaattgaataataagattaaagaaagccctctttataatcaaagcaatactttggaacttataaaagaaacacttcctatgattatgttcattgggttatataaatttacagataaatccttgaacctaatcaattatagaggagtatatcaaaaagatgactctgtttttaagtttaaaccagaggaacaatatttaaattcaatactagcacaccaagtttttagtattaatgaatcattactattatcaaaagttcaaccaaagggccaagcgggaactgaaattgtaattccaacagggaataaaaatttttgtattttactcgttaatggactatttaatattaaatctcaaaaattaaatgaattagaaattctaataaagaaattattagttaaaactgttaattaa

SEQ ID NO: 67 - DNA, 1038 bp, Lactococcus lactis
  source 1..1038: mol_type = other DNA
atgataaatacgagcacagaagacacccagaaagaatactattgtctggaaattgatagagaaaatactatgaaatcattcatgaattttgatagtatagtatctgaactaaaaaatcctagtgaatttaataaagcaagtgtagttgtaaacgaagaaaataaaaaggaagcagaagcttttttgttaaatcataattattacgcattttctatttataaaaaaactttgccagtgagggataaccaaatttttgactttcgagattgcgttgaagtctatgagtttgattcgtttttaagaaatagtttacaaaaatttactggctatattgaaaatttagttaaagcaagtattattaactcactttgttcaaattatagtggtgaacttcaaaaagctcaatgttatttagacccatttatttataaaaatgaagaaattgctgaagatacaataagacatttcggtaggagagcattcataagtaaccaagccttaactattaaacatcatatagataatagaaacggacatatccctttgtgggttattcttcctgaacttacatttggggaaacaacacatttcattgaaagtttaaaagatgagtatcgagaaatgtggatatccgacttattcctgaaaaattcatattataatggagtaaaaggattagaccagcatattaaaaacgttgctcattcatggattactgctagttgggcattaagaaatcgatgtgctcattatagtagagtatatggattaaattttaacattggtaatccaagattttatacagttactttaagagaattaaaagatacaaacaaaaagaagagtgataaccgtgatttatttgcgtttatgttagctatcaagaatgttttgatttgccatgataatagtattcagttagaatggaatgcctttattgattcaattaaagagaaacttgataatagtgaaattgttttaccgtataaaatgggattccctgaagggtatattaaatatttaaaaatagatttatga

SEQ ID NO: 68 - protein, 41 aa, Lactococcus lactis
MRKLTSIGAIALRGISLSGYSQDKDTAETISTTQSTVRNLI

SEQ ID NO: 69 - protein, 255 aa, Lactococcus lactis
METIIKNKKIAIIIAIIVVLGTILLGAYHQSVNRYSSKIAVQIDNVEKLSKNNFAPQNHVNDYGQWIVLAKNANNKSNLFQMIKMTREYDKKFSKDESEAKKNEKKLTDEVNNKWIKIYTQSMLKASESESYGNKITKVWRDAIFENYATVNGKHYYDFKDAIHAFQTQYSNEFSELNSFFQDNIDSANNLSELIRKYFPEKSNRADDLEKFAQDVFNFNDEAINPSGSYSTRSSKLSELDTKALSSSKTVGMRF

SEQ ID NO: 70 - protein, 427 aa, Lactococcus lactis
MAGKKNNATPSWSGYNHQGQVGIFLALNELNKLIQDNISHEDYSVQFEKEDGEDIDIVHSNKVISRHQVKAKTTSKNLNDYSDVLTGFNIDGVDKNFRYLHTICEVIGFGLPKEEFEKLKNKPKYIPNSLNIKLYKYPDGIMYCELNCNKESKIDTFCKKEIINILKKLNSSLKDDDEHIEETLFGLKDLLCSKIRKAHEKGKEEHPIISFQEIYYFVTSTQKRERQSIHRAKTLFKLFWNESLNDVVDDTDDVDDTIIIKILNLADDDFQRLLIDLHPDRNISQLQKGNNIDNLIDENNFKYILYSFIIWCKKENFILDNLYYQTNQDSFRLSLINVPKPAAGDVKKRIMKNIKFIRASFDTKYLINMHIDGTRFFEDKPTHEEGKEKLKSGLVGKRADSIFSSNLEYIDCVNTIEKLREDQHNDV

SEQ ID NO: 71 - protein, 242 aa, Lactococcus lactis
MMYNFIKQIFEKNDYHQYVNIIELWKNSNEFFILQEYDKEELQSSSKIDENFFTCVQTNNIVSEFEKIKDNKIKKNTSLFIIVKVKNLEKEYKRLKNITMRIEEDEYYFRKYVIFYTEDGLSKLKPDAHSLLDYIQTEVPKTTSSVGNNLFDTFEENMFFDDAYFIAMQLIIKLTFISLPHSDKYFETVEDKIKTCIELKGLIDKENQVNQVLKLFSDNNFKEKLEDETSGLLENLNKILGE

SEQ ID NO: 72 - protein, 883 aa, Lactococcus lactis
MKIKKILLYNFKNFRNKTVIDFSDGITFLVGPNGFGKTTIFDAIELGLTGRISRTNKVTGENISYNKPFFQNEIGHPVVIKLWLEKINGEKLVIVRRLMNSSTDGKKAFAPLKSTNQFNLFSQEQINNSSFKSIDNIDTLQPINQSSIDSFLGIEGKCKIENIFNLFNYIQQEETTFFLKQTEQRRSDLLSFLVKTDEVEEKIGKIDKITRVIKGKLDILTEKKKKFTKRELADIPYQRLFSHREFAFDISNPFSYENIDQLPIYKNNIQNIINFKQNFSIPEYKRKIEVEHKMKEIDIDRDIEQAFYYSILAPIIRRPNYHWQWEKYIVENPILFEYVLLENYLQKYDTIVRNHERRKQLNDYRNKLSEELSDMTEKSLNYVQNDSLANDFELLKSQFIQYRTLRSQASQIDKNLSDLKQLRNNLDKQFDELRKHQHVDDNRCPFCNYPYGSFDELKLAYDNYKDYLTSISSQNSQDLQEAQVFIDSIITQIKQKISNELDELGVEVDKQILEKLQELRNQKSKYSVNIDSFKNFIRSYTTILPYELVNLSFTIYNQQYKLRLQEFDSKLEIESDIYTLLDIKKLENTEQKFEELRSEYSGLNFETFQLEPTSNHKISKSMVDSQLYKLREQLKSEVNKVYSINHNLISDPNIILINYFNKDLEHLENISIQDLELKKSYLDKQSNLVQNQQIRILSSEIRILNQTCEQLEEINNVYKAEVKQFKIDIVKQLRIPFFIYSAKMLQNYQQGLGIFLTYKKATENSENKAIIRFKSDPENDHDAMNQLSTGQLAVVSLAFTLALNTMFKLSDNLNFLMIDDPIQDMDAMNVLSFIEILRHGIIDKYQIILSTYSDQNALFMGYKFVNANFLVDIDYKNIRELQG

SEQ ID NO: 73 - protein, 359 aa, Lactococcus lactis
MKKYTIGRNEKCFCGSDIKFKSCCMRKKDLLERESIDAESFNKLYSKGRKQARFRECHHPVKNECSEKIISAHSIQNNKILSKIADDGVVLMPIPKNLNFNEQTEYGRKEATTFTGFCNEHDKYTFRPIEDKDFEGSEEQVFLHIYRAFVLELHRKEEGHRFYQYLFSMMPSIARNSETMKQLWVSKIGHDDLLIEKEEFDKAIIDNKYNCLTYFVWEFEGESNFAATGMEEPRFDYSGLRIQDLEDFDIPAKHIYYTIFPDNGKMYFIIAWLKSNDELFCTIGEKLGSINEEEKKNFVNNVVPSTTENLVINPSSWGAMKRKEKENFNSIFQGKEVMLELLGKLVDRFSYPGYDLFKL

SEQ ID NO: 74 - protein, 212 aa, Lactococcus lactis
MEYDLEFLKNFDGNILPSQINISDLVNVTEKHVSQFRYFYSSDKSEFPEIIIEFKNLHIPHLLGLSKDHHLNLSTYQAREIINNLKRDWNLEYLKSSDEQWFAENKDKLVGVLLLYQLLHVQNCRVLTPLYIINSNFGRRFKRDNVYFVILRSTNNKEYTIELAPMKNHDNVFIPKSLKINDTHVHKCSEISLTFLRSERIKYDKKRGKGRK

SEQ ID NO: 75 - protein, 286 aa, Lactococcus lactis
MRKEKTFKKFYFWSIKFWIPILVIVFVPILVSCLSSSGLTFKEVNGVANTNYYTRAIYQLVPNRKFSTYIIIWFLTLVITLTSIRKMKSSDTFNGNNSIYFSDCYCYLWIAANILGYKKFQLISVSLPLQFELVINEVFSEFIVETSLTPYEKFDGDISIKYKNREYTADIMNVLICDSYNIRENDIAADFNSYPVLKISSSKMDDGIRYDNPKLVRRVREEMNKVSKQYRELNIFLTTNPKNTLKIIGSSFMASDRFGFEKISVISMDNDRIYKKAKVIYQNNKN

SEQ ID NO: 76 - protein, 383 aa, Lactococcus lactis
MEKKELLNKIYCELADSLNISDTMTQEIISSYKSVGSYLGNLEENLNIHIYPQGSMALGTLVRPIAEDKEGDYDVDLVCLLKNGGNLPAKDIKQIIGKRLSEGERYKSKLDAEGKRCWTLKYSDFHMDILPSVPKISTLPNPSRNVTQIRLTHKENDSYFNRYSDPKAYRKWFTKQMEVSFSKQREIIARNKKVEIEEVKLFDVRTPLQMTIQILKRHRDIMFSGRDNKPISIIITTLAAKSYSGETNLYEAMEKVLQDMDKHIEFNNGKAVICNPTMDSENFADKWEEFPEKQQAFFEWLNKARKDIIQEPLEFAGGMGTMKNRMRELFGSKAVNQTFDTYEEKNIKEKNKGNLGVTKEGNIGSIASSNIIAPLSTHTFYGK

SEQ ID NO: 77 - protein, 155 aa, Lactococcus lactis
MVSKKYYPRKKILTPIQQFSLVKQNFSKAKIINNQGKLHLYISLQPSVFSPSYPIEIIEYSRGKYDTWLIGNIQKIDNPKFPHKYEVDKKNNKVRLCLYHPNRGEWTKDQEIYNTLIPWTCEWLYYYELWLDDGKWRGGGEHPRLNDRKRPRRNN

SEQ ID NO: 78 - protein, 537 aa, Lactococcus lactis
MRIDDFLSKISFETSTWDDWGFRQTANITFKEESGDKNFLVKICPNTNENFERIKANQVANEEKNFFWGQTQEYYEFILDYLNDEDDRNRWFKLTGDIANQGQGFVKYYETMKFKYDPELFESKENDRFIAELKMFEDSFFRDQTFNEWTDLLKSLHRLTVNKSFLSDYKFTVKKNSQDILNVDVKPGDNISIKLDNVSLPLSFSNNVFCIIGDNGSGKTSFIRELSKSILDISSELVIENIEFDTQEDANIINKILYCSFSPFDEKINIAHEADSNDEKKFEYIGLLNYDVGNKTIGEEITKDILESLMSVKNNQNNKYLLWLDAMERLSFEKWTYEIVSIFENDLFRSDSEKTEEGVTTSRYIYKNYNETKNKIKKLSSGQKIFLLTITKLVLKLTERTILFIDEPELFLHPPMVKTYVRIISDLVSYVNGLGFIITHSPITLQEFPNDCIKVAYRNQLGEYLIKPINFKTFGENINIINDKVFNIGLQQSGYYNLIESLKYSEDGEKKLKKILNFSGSEAKLLIKLFLEKNEKE

SEQ ID NO: 79 - protein, 443 aa, Lactococcus lactis
MTLTIKDILEVLRDEVVEFVDFIGDGEDESFNQVVDLFSAYEKKYMDEYESLSNLPVISEDDINANFATIKNYYPEYYKGHALYKYREIMESLVEMRCPICDCSFAYSQVTLDHIIPKKKYPYYSITPINLVPTCYNCNMRKNDGTPSKILHPYFHDFITFDYLTVTLEVNEKEPTESIINLGFTELNSDDVNEINKVESIKANIDLYKLRQKYTDIINISFSKLIDEFQKVVSIQNDVYSINELKKYFKLMDIFMDSNDYNYVDEDFLRHLCIVEITQNTHFLSCLAEKLNIFVDYDTQIVESMEKLNNKIKESPLYNQSNTLELIKETLPMIMFIGLYKFTDKSLNLINYRGVYQKDDSVFKFKPEEQYLNSILAHQVFSINESLLLSKVQPKGQAGTEIVIPTGNKNFCILLVNGLFNIKSQKLNELEILIKKLLVKTVN

SEQ ID NO: 80 - protein, 345 aa, Lactococcus lactis
MINTSTEDTQKEYYCLEIDRENTMKSFMNFDSIVSELKNPSEFNKASVVVNEENKKEAEAFLLNHNYYAFSIYKKTLPVRDNQIFDFRDCVEVYEFDSFLRNSLQKFTGYIENLVKASIINSLCSNYSGELQKAQCYLDPFIYKNEEIAEDTIRHFGRRAFISNQALTIKHHIDNRNGHIPLWVILPELTFGETTHFIESLKDEYREMWISDLFLKNSYYNGVKGLDQHIKNVAHSWITASWALRNRCAHYSRVYGLNFNIGNPRFYTVTLRELKDTNKKKSDNRDLFAFMLAIKNVLICHDNSIQLEWNAFIDSIKEKLDNSEIVLPYKMGFPEGYIKYLKIDL

SEQ ID NO: 81 - DNA, 48 bp, synthetic construct
  source 1..48: mol_type = other DNA
agcagcggatccaggaggcactcaccatgagaaagcttacaagtattg

SEQ ID NO: 82 - DNA, 33 bp, synthetic construct
  source 1..33: mol_type = other DNA
agcagcgtcgacttagaacctcattccaacagt

SEQ ID NO: 83 - DNA, 48 bp, synthetic construct
  source 1..48: mol_type = other DNA
agcagcggatccaggaggcactcaccatggctggaaaaaagaataatg

SEQ ID NO: 84 - DNA, 34 bp, synthetic construct
  source 1..34: mol_type = other DNA
agcagcgtcgacttagccttgtaattcccttata

SEQ ID NO: 85 - DNA, 53 bp, synthetic construct
  source 1..53: mol_type = other DNA
agcagcctgcagaggaggcactcaccatgaaaaagtatactattggtagaaat

SEQ ID NO: 86 - DNA, 37 bp, synthetic construct
  source 1..37: mol_type = other DNA
agcagcggtaccactccttctataagtttgaacaatt

SEQ ID NO: 87 - DNA, 34 bp, synthetic construct
  source 1..34: mol_type = other DNA
agcagcggatcctcaactagcaattcgggtaaag

SEQ ID NO: 88 - DNA, 33 bp, synthetic construct
  source 1..33: mol_type = other DNA
agcagcgtcgacagcttattttctgccctttcc

SEQ ID NO: 89 - DNA, 51 bp, synthetic construct
  source 1..51: mol_type = other DNA
agcagcggtaccaggaggcactcaccatgaggaaagaaaaaacttttaaaa

SEQ ID NO: 90 - DNA, 34 bp, synthetic construct
  source 1..34: mol_type = other DNA
agcagctctagatcaattatttcttctaggcctt

SEQ ID NO: 91 - DNA, 46 bp, synthetic construct
  source 1..46: mol_type = other DNA
agcagcctgcagaggaggcactcacctcttcggcttttcagtttct

SEQ ID NO: 92 - DNA, 32 bp, synthetic construct
  source 1..32: mol_type = other DNA
agcagcggtacctcataaatctatttttaaat